SEQUENCE LISTING (2 sequences)

SEQ ID NO: 1 - DNA, 22 bp, Artificial Sequence
  synthetic small inhibitory RNA
accggttaaacaatgcattcat

SEQ ID NO: 2 - DNA, 22 bp, Artificial Sequence
  synthetic small inhibitory RNA
atgaatgcattgtttaaccggc

(19) United States
(12) Patent Application Publication
(10) Pub. No.: US 2010/0292303 A1
(43) Pub. Date: Nov. 18, 2010

(54) GENE EXPRESSION PROFILE FOR PREDICTING OVARIAN CANCER PATIENT SURVIVAL

(76) Inventors: Michael J. Birrer, Boston, MA (US); Tomas A. Bonome, Washington, WA (US); Laurent L. Ozbun, Germantown, MD (US); Samuel Mok, Boston, MA (US)

Correspondence Address: KLARQUIST SPARKMAN, LLP (OTT-NIH), 121 S.W. SALMON STREET, SUITE #1600, PORTLAND, OR, 97204-2988, US

(21) Appl. No.: 12/669,894

(22) PCT Filed: Jul. 19, 2008
(86) PCT No.: PCT/US08/70565

§ 371 Date: Aug. 5, 2010

Related U.S. Application Data
(60) Provisional application No. 60/951,073, filed on Jul. 20, 2007.

(51) Int. Cl. C12Q 1/02; C12Q 1/37; A61K 31/713; A61P 35/00

(52) U.S. Cl. 514/44 A; 435/29; 435/24

(57) ABSTRACT

A gene profiling signature for predicting ovarian cancer patient survival is disclosed herein. The gene signature can be used to diagnosis or prognosis ovarian cancer, identify agents to treat an ovarian tumor, to predict the metastatic potential of an ovarian tumor and to determine the effectiveness of ovarian tumor treatments. Thus, methods are provided for diagnosing and prognosing an ovarian tumor, such as ovarian cancer, in a subject. Methods are also provided for identifying agents that can be used to treat an ovarian tumor, for determining the effectiveness of an ovarian tumor treatment, or to predict the metastatic potential of an ovarian tumor. Methods of treatment are also disclosed which include administering a composition that includes a specific binding agent that specifically binds to one of the disclosed ovarian survival factor-associated molecules and ovarian tumor in the subject.

### CROSS REFERENCE TO RELATED APPLICATION

This application claims the benefit of U.S. Provisional Application No. 60/951,073, filed on Jul. 20, 2007, which is incorporated herein by reference in its entirety.

### FIELD OF THE DISCLOSURE

This disclosure relates to the field of ovarian cancer and in particular, to methods for predicting survival of patients with ovarian cancer.

### BACKGROUND

Ovarian cancer is the fifth most common form of cancer in women in the United States, accounting for three percent of the total number of cancer cases and twenty-six percent of those occurring in the female genital tract. The American Cancer Society estimates that 15,310 deaths were caused in women living in the United States in 2006. A large majority of women who die of ovarian cancer will have had serous carcinoma of the ovarian epithelium, a condition that occurs in sixty percent of all cases of ovarian cancer (Boring et al., Cancer J. Clin. 44: 7-26, 1994).

Women with ovarian cancer are typically asymptomatic until the cancer has metastasized. As a result, most women with ovarian cancer are not diagnosed until the cancer has progressed to an advanced and usually incurable stage (Boente et al., Curr. Probl. Cancer 20: 83-137, 1996). Survival rates are much better in women diagnosed with early-stage ovarian cancers, with about ninety percent of these women still alive five years after diagnosis.

Treatment of ovarian cancer typically involves a variety of treatment modalities. Generally, surgical intervention serves as the basis for treatment (Dennis S. Chi & William J. Hoskins, Primary Surgical Management of Advanced Epithelial Ovarian Cancer, in Ovarian Cancer 241, Stephen C. Rubin & Gregory P. Sutton eds., 2d ed. 2001). Treatment of serous carcinoma often involves cytoreductive surgery (hysterectomy, bilateral salpingo-oophorectomy, omentectomy, and lymphadenectomy) followed by adjuvant chemotherapy with paclitaxel and either cisplatin or carboplatin (Eltabbakh & Awtrey, Expert Op. Pharmacother. 2(10): 109-24, 2001).

Despite a clinical response rate of 80% of advanced ovarian cancers (stages III/IV) to primary treatment with surgery and chemotherapy, most subjects experience tumor recurrence within two years of treatment. The overwhelming majority of subjects will eventually develop chemoresistance and die as a result of their cancer. Though most subjects die within two years of diagnosis, a subset of subjects, even with clinically and morphologically indistinguishable disease, develop a more chronic form of ovarian cancer, and may survive five years or more with treatment. Currently, clinicians lack adequate prognostic tools to predict the disease's clinical course at the time of initial diagnosis and possess few alternative treatment regimens beyond conventional first-line chemotherapeutic agents.

### SUMMARY OF THE DISCLOSURE

Advanced papillary serous tumors of the ovary are responsible for the majority of ovarian cancer deaths, but little is known about the molecular determinants modulating patient survival. Disclosed herein is a prognostic gene expression signature that can be used to predict survival of a subject with ovarian cancer, such as advanced stage ovarian cancer. In one example, the gene expression signature includes 200 genes whose expression is associated with poor survival in subjects with advanced ovarian cancer.

Methods are disclosed for predicting a clinical outcome in a subject with an ovarian tumor, such as advanced stage papillary serous ovarian cancer. In an example, the methods include detecting expression of at least one ovarian survival factor-associated molecule listed in Table 1, Table 2, FIG. 1B, or combinations thereof (such as at least 2, at least 3, at least 5 or at least 10 of such molecules) in a sample obtained from the subject with the ovarian tumor. The methods also can include comparing expression of the at least one ovarian survival factor-associated molecule in the sample obtained from the subject with the ovarian tumor to a control, wherein an alteration in the expression of the at least one ovarian survival factor-associated molecule indicates that the subject has a decreased chance of survival. For example, an alteration in the expression, such as an increase in the expression of microfibril-associated glycoprotein 2 (MAGP2), protein tyrosine phosphatase receptor D (PTPRD), matrix metallopeptidase 13 (MMP13), stanniocalcin 2 (STC2), chemokine (C-C motif) receptor-like 1 (CCRL1), klotho beta (KLB) or combination thereof indicates a poor prognosis, such as a decreased chance of survival. In one example, a decreased chance of survival includes a survival time of equal to or less than a year. Alterations in the expression can be measured using methods known in the art, and this disclosure is not limited to particular methods. For example, expression can be measured at the nucleic acid level (such as by real time quantitative polymerase chain reaction or microarray analysis) or at the protein level (such as by Western blot analysis).

In some examples, the method includes determining the metastatic potential of an ovarian tumor in a subject by detecting expression of at least one ovarian survival factor-associated molecule in a sample obtained from a subject with an ovarian tumor. The at least one ovarian survival factor-associated molecule can be involved in promoting angiogenesis, such as cell proliferation, cell motility or tube formation. The method can further include comparing expression of the at least one ovarian survival factor-associated molecule in the sample obtained from the subject having the ovarian tumor to a control. An alteration in the expression, such as an increase in the expression of the at least one ovarian survival factor-associated molecule involved in promoting angiogenesis, indicates that the subject has an ovarian tumor with increased metastatic potential.

The disclosed prognostic gene expression signature also has implications for the treatment of ovarian cancer. For example, the ovarian survival factor-associated molecules identified by the gene profile signature can serve as targets for specific molecular therapeutic molecules that can treat ovarian cancer. Thus, methods are disclosed for identifying agents that can be used in treating an ovarian tumor.

In an example, the method of identifying an agent for treating an ovarian tumor includes contacting an ovarian tumor epithelial cell with one or more test agents under conditions sufficient for the one or more test agents to alter the activity of at least one ovarian survival factor-associated molecule listed in any of Tables 1 and 2. The method can also include detecting the activity of the at least one ovarian survival factor-associated molecule in the presence and absence of the one or more test agents. The activity of the at least one ovarian survival factor-associated molecule in the presence of the one or more test agents can be compared to a control, such as a value representing the activity in the absence of such agents, to determine if there is differential expression of the at least one ovarian survival factor-associated molecule. Differential expression of the ovarian survival factor-associated molecule indicates that the one or more test agents are of use to treat the ovarian tumor and can be selected for further analysis.

The disclosed methods can further include administering to the subject a therapeutically effective treatment to alter the expression of at least one of the disclosed ovarian survival factor-associated molecules. In an example, the treatment includes administering a therapeutically effective amount of an agent that decreases biological activity. In particular examples, the agent is a specific binding agent that preferentially binds to and decreases expression of at least one of the ovarian survival factor-associated molecules listed in Tables 1 or 2, such as MAGP2, PTPRD, MMP13, STC2, CCRL1 or KLB, which are upregulated in subjects with a poor prognosis. In other particular examples, ovarian tumor growth is reduced or inhibited by the specific binding agent preferentially binding to and/or altering expression of one of the ovarian survival factor-associated molecules listed in any of Tables 1 or 2 which are involved in angiogenesis, such as molecules involved in cell proliferation, cell motility or cell adhesion, such as MAGP2 or CCRL1.

Also provided are methods of determining the effectiveness of an agent for the treatment of an ovarian tumor in a subject with the ovarian tumor. In one example, the method includes detecting expression of an ovarian survival factor-associated molecule in a sample from the subject following treatment with the agent. The expression of the ovarian survival factor-associated molecule following treatment can be compared to a control. An alteration in the expression of the ovarian survival factor-associated molecule following treatment can indicate that the agent is effective for the treatment of an ovarian tumor in the subject, such as papillary serous ovarian cancer. In a specific example, the method includes detecting and comparing the protein expression levels of the ovarian survival factor-associated molecules. In other examples, the method includes detecting and comparing the mRNA expression levels of the ovarian survival factor-associated molecules.

The foregoing and other features of the disclosure will become more apparent from the following detailed description of several embodiments that proceeds with reference to the accompanying figures.

### BRIEF DESCRIPTION OF THE FIGURES

FIG. 1A includes a table of the genes provided in FIG. 1A with a Cox score >10.

FIG. 1B is a Kaplan-Meier plot of samples presented in FIG. 1A.

FIG. 1C is a Kaplan-Meier plot generated using quantitative real-time-PCR (qRT-PCR).

FIG. 2A is a schematic drawing illustrating the signaling pathway of select differentially regulated genes identified in 53 microdissected serous tumors.

FIG. 2B is a bar graph showing the mean-fold change in select differentially regulated genes using SYBR-green based qRT-PCR.

FIG. 3A is a digital image of the comparative genomic hybridization (CGH) analyses demonstrating an amplification in the MAGP2 locus and its products in serous ovarian tumors.

FIG. 3B is a bar graph illustrating the fold change in MAGP2 as detected by qPCR and microarray analysis.

FIG. 3C is a Kaplan-Meier plot estimating survival using MAGP2 mRNA expression as an indicator.

FIG. 3D is a Kaplan-Meier plot estimating survival using MAGP2 protein expression as an indicator.

FIG. 3E is a digital image showing select examples of typical MAGP2 staining from a tissue microarray (TMA) containing 81 papillary serous ovarian cancer sections. Staining ranged from high-level encompassing the majority of the section (subpanel A), moderate staining (subpanel B), and low-level (subpanel C).

FIG. 3F is a bar graph illustrating the relationship between MAGP2 protein expression levels among chemotherapy responders and non-responders.

FIG. 4A is bar graph illustrating the mean-fold change in MAGP2 expression in RNA isolates obtained from 2 normal HOSE cultures and 12 ovarian cancer cell lines as measured by qRT-PCR.

FIG. 4B includes graphs illustrating flow cytometry analysis of αᵥβ₃ cell surface receptor levels in select high and low MAGP2 expressing cell lines using monoclonal antibodies against CD51/61 or IgG1 isotype control.

FIG. 4C is a bar graph illustrating the mean-fold change in A224 ovarian cell adhesion in the presence of purified recombinant MAGP2 (recMAGP2).

FIG. 4D is a bar graph illustrating the mean-fold change in UC107 cell adhesion when cultured in the presence of recMAGP2.

FIG. 4E is a bar graph illustrating an increase in OVCA429 cell viability (as indicated by an increase in fluorescence) in cells treated with higher concentrations of recMAGP2.

FIG. 5A is a bar graph illustrating a mean-fold change in human umbilical vein endothelial (HUVE) cells adhesion when cultured in the presence of recMAGP2 alone or following anti-αᵥβ₃ integrin antibody pre-treatment.

FIG. 5B is a bar graph illustrating a mean-fold change in HUVE cell motility in response to increasing concentrations (10 ng/ml, 50 ng/ml and 100 ng/ml) of recMAGP2 protein.

FIG. 5C is a bar graph illustrating a mean-fold change in HUVE cell motility in response to recMAGP2 treatment (100 ng/ml) following anti-αᵥβ₃ integrin antibody pre-treatment.

FIG. 5D is a bar graph illustrating a mean-fold change in HUVE cell invasion into matrigel in response to recMAGP2 treatment (4.5 ng/ml).

FIG. 5E is a bar graph illustrating a mean-fold change in HUVE cell survival with recMAGP2 treatment.

FIG. 6A is a schematic illustrating the signaling events mediating the effects of MAGP2 on HUVE cells

FIGS. 6B-D are graphs illustrating time course changes in [Ca²⁺] levels as measured by Fluo-4 emission intensity. FIG. 6B shows changes in [Ca²⁺] levels caused in a single 100 ng/ml MAGP2 induced HUVE Cell, FIG. 6C changes induced by recMAGP2 alone, and FIG. 6D changes induced by recMAGP2 following pre-treatment with a competitor peptide.

FIG. 7 is a table illustrating the correlation of MAGP2 expression with CD34 positive microvessel density in 30 late-stage high-grade serous ovarian adenocarcinomas.

FIG. 8A is a digital image illustrating decreased MAGP2 expression and CD34 in ovarian tumors following treatment with MAGP2 siRNA.

FIG. 8B is a graph showing the significant difference in the weight of an ovarian tumor treated with MAGP2 siRNA and the control group as determined by Mann-Witney U test.

### DETAILED DESCRIPTION OF SEVERAL EMBODIMENTS

There is a need for diagnostic classifiers that can reliably stratify ovarian tumor subjects for therapy, as well as new targets for therapeutic intervention of ovarian tumors. Large-scale transcription profiling can identify differentially expressed genes and molecular signatures in numerous biological systems, including ovarian cancer (Alizadeh et al., Nature 403: 503-511, 2000); Bonome et al., Cancer Res. 65: 10602-10612, 2005; DeRisi et al., Science 278: 680-686, 1997; Golub et al., Science 286: 531-537, 1999; and Zorn et al., Clin. Cancer Res. 11: 6422-6430, 2005). Efforts to derive clinical predictors for survival in ovarian cancer from gene expression data have focused on discrete subject groups clustered at either end of the survival spectrum (Berchuck et al., Clin. Cancer Res. 11:3686-96, 2005; and Lancaster et al., J. Sco. Gynecol. Investig. 11:51-9, 2004). Yet, expression patterns identified in this manner may not adequately differentiate the majority of subjects who will succumb at an intermediate endpoint. In addition, the evaluation of undissected tumor isolates may introduce erroneous data attributable to varying amounts of intervening stroma and lymphocytic infiltrate.

It is shown herein that correlating survival, as a continuous variable, with gene expression can provide a predictive signature for advanced stage serous ovarian cancer subjects who are likely to develop aggressive, recurrent disease, and identify biologically relevant targets of clinical importance in a large proportion of patients. Furthermore, analysis of homogenous tumor epithelial specimens can ensure the expression signatures are specific to the cell type of interest. Therefore, disclosed herein is a prognostic gene expression signature that can be used to predict survival of a subject with an ovarian tumor, such as ovarian cancer. In an example, the gene expression signature includes a set of 200 genes whose expression is associated with poor patient survival in subjects with an ovarian tumor, such as advanced ovarian cancer.

To characterize the disclosed prognostic gene expression signature, genes possessing the highest correlation scores were confirmed by qRT-PCR and used to recapitulate the Kaplan Meier survival curve observed for the microarray data. Among the validated genes was extracellular microfibril-associated glycoprotein 2 (MAGP2). The protein product of MAGP2 is shown herein to be capable of enhancing ovarian tumor cell survival, as well as promote the motility and survival of endothelial cells in vitro. Combined with the in vitro data, correlation of MAGP2 protein levels with increased tumor microvessel density indicates a pro-angiogenic role for this protein in vivo. Thus, in addition to providing a biological confirmation of the prognostic survival signature, MAGP2 expression can play a role in tumor cell-induced angiogenesis and survival in papillary serous ovarian cancer.

Accordingly, the disclosed gene expression profile also identifies genes and collections or sets of genes that serve as effective molecular markers for angiogenesis in an ovarian tumor, as well as such genes or gene sets that can provide clinically effective therapeutic targets for ovarian cancer. For example, methods are disclosed for reducing or inhibiting an ovarian tumor by targeting ovarian survival factor-associated molecules, such as molecules involved in angiogenesis. In an example, molecules involved in angiogenesis include molecules involved in cell motility, tube formation or cell proliferation, identified by the disclosed gene profile signature. In one example, a therapeutically effective amount of a specific binding agent, such as an antibody or siRNA molecule, is administered to a subject. For example, the specific binding agent can preferentially bind to one or more of the identified ovarian survival factor-associated molecules listed in any of Tables 1, 2, or a combination thereof. As a result, an ovarian tumor, such as ovarian cancer, in the subject is thereby reduced or eliminated. In a particular example, the specific binding agent is an inhibitor, such as a siRNA, specific for one or more of the disclosed ovarian survival factor-associated molecules described in any of Tables 1 or 2 thereby reducing or inhibiting expression of these molecules.

### TERMS AND ABBREVIATIONS

CCRL1 chemokine (C-C motif) receptor-like 1

FAK focal adhesion kinase

HOSE cells human ovarian surface epithelial cells

HUVE cells human umbilical venous endothelial cells

KLB klotho beta

MAGP2 microfibril associated glycoprotein 2

MMP13 matrix metallopeptidase 13

PTPRD protein tyrosine phosphatase receptor D

qRT-PCR quantitative real-time polymerase chain reaction

recMAGP2 recombinant MAGP2

STC2 stanniocalcin 2

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Administration: To provide or give a subject an agent, such as a chemotherapeutic agent, by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Agent: Any protein, nucleic acid molecule, compound, small molecule, organic compound, inorganic compound, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject). In examples, an agent can act directly or indirectly to alter the activity of one or more molecules listed in Table 1 or Table 2. In a particular example, a pharmaceutical agent (such as a siRNA or antibody to any of the molecules listed in Table 1 or Table 2) significantly reduces the expression and/or activity of an ovarian survival factor-associated molecule thereby increasing a subject's survival time. In an example, a “test agent” is any substance, including, but not limited to, a protein (such as an antibody), nucleic acid molecule (such as a siRNA), organic compound, inorganic compound, or other molecule of interest. In particular examples, a test agent can permeate a cell membrane (alone or in the presence of a carrier).

Amplifying a nucleic acid molecule: To increase the number of copies of a nucleic acid molecule, such as a gene or fragment of a gene, for example a region of an ovarian survival factor-associated molecule listed in Table 1 or Table 2. The resulting products are called amplification products.

An example of in vitro amplification is the polymerase chain reaction (PCR), in which a biological sample obtained from a subject (such as a sample containing ovarian cancer cells) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

A commonly used method for real-time quantitative polymerase chain reaction involves the use of a double stranded DNA dye (such as SYBR Green I dye). For example, as the amount of PCR product increases, more SYBR Green I dye binds to DNA, resulting in a steady increase in fluorescence. Another commonly used method is real-time quantitative TaqMan PCR (Applied Biosystems). The 5′ nuclease assay provides a real-time method for detecting only specific amplification products. During amplification, annealing of the probe to its target sequence generates a substrate that is cleaved by the 5′ nuclease activity of Taq DNA polymerase when the enzyme extends from an upstream primer into the region of the probe. This dependence on polymerization ensures that cleavage of the probe occurs only if the target sequence is being amplified. The use of fluorogenic probes makes it possible to eliminate post-PCR processing for the analysis of probe degradation. The probe is an oligonucleotide with both a reporter fluorescent dye and a quencher dye attached. While the probe is intact, the proximity of the quencher greatly reduces the fluorescence emitted by the reporter dye by Förster resonance energy transfer (FRET) through space. Probe design and synthesis has been simplified by the finding that adequate quenching is observed for probes with the reporter at the 5′ end and the quencher at the 3′ end.

Angiogenesis: A physiological process involving the growth of new blood vessels from pre-existing vessels. Angiogenesis can occur under normal physiological conditions, such as during growth and development or wound healing (known as physiological angiogenesis), as well as pathological conditions such as in the transition of tumors from a dormant state to a malignant state (known as pathological angiogenesis). As used herein, pro-angiogenic genes are genes that facilitate angiogenesis, such as angiogenesis in an ovarian tumor. Examples of such genes include TWIST homolog 1 (TWIST1), TWIST homolog 2 (TWIST2), MAGP2, CCRL, glutamyl aminopeptidase (ENPEP), tumor necrosis factor, alpha-induced protein 6 (TNFAIP6), PTPRF interacting protein, binding protein 1 (liprin beta 1; PPIBP1), desmocollin 2 (DSC2), surfactant pulmonary-associated protein D (SFTPD), fibroblast growth factor 18 (FGF18), neural precursor cell expressed developmentally down-regulated 9 (NEDD9), fibroblast growth factor receptor 2 (FGFR2), fibrinogen betachain (FGB), fibronectin leucine rich transmembrane protein 3, (FLRT3), phosphatase and tensin homolog, angiopoietin 2 (ANGPT2), matrixmetallopeptidase 12 (MMP12), matrixmetallopeptidase 13 (MMP13), chromosome 12 open reading frame 9 (C12orf9), protocadherin 10 (PCDH10) and stanniocalcin 2 (STC2), and sterial alpha motif and leucine zipper containing kinase AZK (ZAK).

The complex phenomenon of angiogenesis begins with degradation of the basement membrane by cellular proteases. This allows endothelial cells to penetrate and migrate (process known as cell motility) into the extracellular matrix and then proliferate. In the final stages of this process, the endothelial cells align themselves to form capillary or tubelike structures (process known as tube formation). These new structures then form a network that undergoes significant remodeling and rearrangement before fully functioning capillaries exist. Therefore, angiogenesis can be studied or identified by monitoring tube formation, cell motility, and/or cell proliferation. Angiogenesis is also studied or identified by monitoring cell adhesion.

Antibody: A polypeptide including at least a light chain or heavy chain immunoglobulin variable region which specifically recognizes and binds an epitope of an antigen, such as an ovarian survival factor-associated molecule or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Antibodies of the present disclosure include those that are specific for the molecules listed in Tables 1 or 2.

The term antibody includes intact immunoglobulins, as well the variants and portions thereof, such as Fab′ fragments, F(ab)'₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds RET will have a specific V_{H} region and the V_{L} region sequence, and thus specific CDR sequences. Antibodies with different specificities (such as different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “polyclonal antibody” is an antibody that is derived from different B-cell lines. Polyclonal antibodies are a mixture of immunoglobulin molecules secreted against a specific antigen, each recognizing a different epitope. These antibodies are produced by methods known to those of skill in the art, for instance, by injection of an antigen into a suitable mammal (such as a mouse, rabbit or goat) that induces the B-lymphocytes to produce IgG immunoglobulins specific for the antigen, which are then purified from the mammal's serum.

A “chimeric antibody” has framework residues from one species, such as human, and CDRs (which generally confer antigen binding) from another species, such as a murine antibody that specifically binds an ovarian survival factor-associated molecule.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one example, all the CDRs are from the donor immunoglobulin in a humanized immunoglobulin. Constant regions need not be present, but if they are, they are substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. Humanized immunoglobulins can be constructed by means of genetic engineering (see for example, U.S. Pat. No. 5,585,089).

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called DNA chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from at least one, to at least 2, to at least 5, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect sensitive to ovarian survival factor-associated molecule sequences, such as at least one of those of the sequences listed in Table 1 or Table 2, such as at least 5, at least 7, at least 10, at least 20, at least 30, at least 40, at least 50, at least 60, at least 70, at least 80, at least 90, at least 100, at least 150, or at least 175 sequences listed in Table 1 or Table 2 (for example, 2, 6, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 130, 140, 150, 170, 180, 190 or 200 of those listed). In an example, the array is a commercially available such as a U133 Plus 2.0 oligonucleotide array from Affymetrix (Affymetrix, Santa Clara, Calif.).

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Protein-based arrays include probe molecules that are or include proteins, or where the target molecules are or include proteins, and arrays including nucleic acids to which proteins are bound, or vice versa. In some examples, an array contains antibodies to ovarian survival factor-associated molecule proteins, such as any combination of those sequences listed in Table 1 or Table 2, such as at least 2, least 5, at least 7, at least 10, at least 20, at least 30, at least 40, at least 50, at least 60, at least 70, at least 80, at least 90, at least 100, at least 150, or at least 175 sequences listed in Table 1 or Table 2 (for example, 2, 6, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 130, 140, 150, 170, 180, 190 or 200 of those listed).

Binding or stable binding: An association between two substances or molecules, such as the hybridization of one nucleic acid molecule to another (or itself), the association of an antibody with a peptide, or the association of a protein with another protein or nucleic acid molecule. An oligonucleotide molecule binds or stably binds to a target nucleic acid molecule (such as any of those listed in Tables 1 and 2) if a sufficient amount of the oligonucleotide molecule forms base pairs or is hybridized to its target nucleic acid molecule, to permit detection of that binding. “Preferentially binds” indicates that one molecule binds to another with high affinity, and binds to heterologous molecules at a low affinity.

Binding can be detected by any procedure known to one skilled in the art, such as by physical or functional properties of the target:oligonucleotide complex. For example, binding can be detected functionally by determining whether binding has an observable effect upon a biosynthetic process such as expression of a gene, DNA replication, transcription, translation, and the like.

Physical methods of detecting the binding of complementary strands of nucleic acid molecules, include but are not limited to, such methods as DNase I or chemical footprinting, gel shift and affinity cleavage assays, Northern blotting, dot blotting and light absorption detection procedures. For example, one method involves observing a change in light absorption of a solution containing an oligonucleotide (or an analog) and a target nucleic acid at 220 to 300 nm as the temperature is slowly increased. If the oligonucleotide or analog has bound to its target, there is a rapid increase in absorption at a characteristic temperature as the oligonucleotide (or analog) and target disassociate from each other, or melt. In another example, the method involves detecting a signal, such as a detectable label, present on one or both nucleic acid molecules (or antibody or protein as appropriate).

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

In some examples, an antibody specifically binds to a target with a binding constant that is at least 10³ M⁻¹ greater, 10⁴M⁻¹ greater or 10⁵ M⁻¹ greater than a binding constant for other molecules in a sample. In some examples, a specific binding reagent (such as an antibody (e.g., monoclonal antibody) or fragments thereof) has an equilibrium constant (Kd) of 1 nM or less. For example, a specific binding agent binds to a target, such as MAGP2 protein with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, N.J.

Biological activity: The beneficial or adverse effects of an agent on living matter. When the agent is a complex chemical mixture, this activity is exerted by the substance's active ingredient or pharmacophore, but can be modified by the other constituents. Activity is generally dosage-dependent and it is not uncommon to have effects ranging from beneficial to adverse for one substance when going from low to high doses. In one example, the agent significantly reduces the biological activity of the one or more ovarian survival factor-associated molecules (such as those listed in Tables 1 and 2) which reduces or eliminates ovarian cancer, such as by reducing or inhibiting angiogenesis.

Cancer: The “pathology” of cancer includes all phenomena that compromise the well-being of the subject. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc. “Metastatic disease” refers to cancer cells that have left the original tumor site and migrate to other parts of the body, for example via the bloodstream or lymph system.

Chemotherapeutic agent or Chemotherapy: Any chemical agent with therapeutic usefulness in the treatment of diseases characterized by abnormal cell growth. Such diseases include tumors, neoplasms, and cancer. In one example, a chemotherapeutic agent is an agent of use in treating ovarian cancer, such as papillary serous ovarian cancer. In one example, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Exemplary chemotherapeutic agents used for treating ovarian cancer include carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone and vinorelbine. Combination chemotherapy is the administration of more than one agent to treat cancer.

Chemokine (C-C motif) receptor-like 1 (CCRL1): The protein encoded by this gene is a member of the G protein-coupled receptor family, and is a receptor for C-C type chemokines This receptor also binds dendritic cell- and T cell-activated chemokines including CCL19/ELC, CCL21/SLC, and CCL25/TECK, and is involved in chemotaxis. In particular examples, expression of CCRL1 is altered in an ovarian tumor, such as increased. The term CCRL1 includes any CCRL1 gene, cDNA, mRNA, or protein from any organism and that is CCRL1 and is expressed in an ovarian tumor.

Nucleic acid and protein sequences for CCRL1 are publicly available. For example, GENBANK® Accession Nos.: NM_{—}174265, NM_{—}145700, AY221094, NM_{—}016557, and NM_{—}178445 disclose CCRL1 nucleic acid sequences, and GENBANK® Accession Nos.: AAH95501, NP_{—}848540, and NP_{—}057641 disclose CCRL1 protein sequences, all of which are incorporated by reference as provided by GENBANK® on Apr. 13, 2007.

In one example, CCRL1 includes a full-length wild-type (or native) sequence, as well as CCRL1 allelic variants, fragments, homologs or fusion sequences that retain the ability to be expressed at increased levels in an ovarian tumor and/or modulate an activity of an ovarian tumor, such as vascular growth. In certain examples, CCRL1 has at least 80% sequence identity, for example at least 85%, 90%, 95%, or 98% sequence identity to CCRL1. In other examples, CCRL1 has a sequence that hybridizes to AFFYMETRIX® Probe ID No. 203439_s_and retains CCRL1 activity (such as the capability to be overexpressed in an ovarian tumor and/or modulate tumor and/or vascular growth, such as by chemotaxis).

Comparative genomic hybridization (CGH): A molecular-cytogenetic method for the analysis of copy number changes (gains/losses) in the DNA content of cells, such as tumor cells. The method is based on the hybridization of fluorescently labeled tumor DNA (such as, Fluorescein—FITC) and normal DNA (such as, Rhodamine or Texas Red) to normal human metaphase preparations. Using methods known in the art, such as epiflourescence microscopy and quantitative image analysis, regional differences in the fluorescence ratio of tumor versus control DNA can be detected and used for identifying abnormal regions in the tumor cell genome. CGH detects unbalanced chromosomes changes. Structural chromosome aberrations, such as balanced reciprocal translocations or inversions, are not detected, as they do not change the copy number.

In one example, CGH includes the following steps. DNA from tumor tissue and from normal control tissue (reference) is labeled with different detectable labels, such as two different fluorophores. After mixing tumor and reference DNA along with unlabeled human cot 1 DNA to suppress repetitive DNA sequences, the mix is hybridized to normal metaphase chromosomes or, for array- or matrix-CGH, to a slide containing hundreds or thousands of defined DNA probes. The (fluorescence) color ratio along the chromosomes is used to evaluate regions of DNA gain or loss in the tumor sample.

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence (such as any of the molecules listed in Table 1 or 2) under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as a ovarian survival factor-associated molecule, for example any of the genes listed in Table 1 or 2) to achieve detectable binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Contacting: Placement in direct physical association, including both a solid and liquid form. Contacting an agent with a cell can occur in vitro by adding the agent to isolated cells or in vivo by administering the agent to a subject.

Control: Samples believed to be normal (in that they are not altered for the desired characteristic, for example a sample from a subject who does not have cancer, such as ovarian cancer) as well as laboratory values, even though possibly arbitrarily set, keeping in mind that such values can vary from laboratory to laboratory.

Cox hazard ratio: The ratio of survival hazards for a one-unit change in logarithmic gene expression levels. This ratio is derived from the Cox proportional hazards model, which measures the instantaneous force of mortality at any time conditional on having survived until that time. For hazard ratios greater than 1, increased gene expression is associated with a reduction in overall patient survival. The magnitude of the ratio indicates the degree of impact a one-unit increase in the logarithmic gene expression has on patient survival. Thus, a larger value has a greater effect on overall survival.

Decrease: To reduce the quality, amount, or strength of something. In one example, a therapy decreases a tumor (such as the size of a tumor, the number of tumors, the metastasis of a tumor, or combinations thereof), or one or more symptoms associated with a tumor, for example as compared to the response in the absence of the therapy (such as a therapy administered to affect tumor size by inhibiting angiogenesis via administration of a binding agent capable of binding to one or more of the ovarian survival factor-associated molecules listed in Tables 1 and 2). In a particular example, a therapy decreases the size of a tumor, the number of tumors, the metastasis of a tumor, or combinations thereof, subsequent to the therapy, such as a decrease of at least 10%, at least 20%, at least 50%, or even at least 90%. Such decreases can be measured using the methods disclosed herein. In additional examples, the presence of at least one of the disclosed ovarian survival factor-associated molecules decreases a subject's chance of survival.

Deoxyribonucleic acid (DNA): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Detecting expression of a gene product: Determining of a level expression in either a qualitative or quantitative manner can detect nucleic acid or protein. Exemplary methods include microarray analysis, RT-PCR, Northern blot, Western blot, and mass spectrometry.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy.

Differential or alteration in expression: A difference or alteration, such as an increase or decrease, in the conversion of the information encoded in a gene (such as an ovarian survival factor-associated molecule listed in Table 1 or 2) into messenger RNA, the conversion of mRNA to a protein, or both. In some examples, the difference is relative to a control or reference value or range of values, such as an amount of gene expression that is expected in a subject who does not have ovarian cancer. Detecting differential expression can include measuring a change in gene expression.

Downregulated or inactivation: When used in reference to the expression of a nucleic acid molecule, such as a gene, refers to any process which results in a decrease in production of a gene product (such as one or more of those listed in Tables 1 and 2). A gene product can be RNA (such as mRNA, rRNA, tRNA, and structural RNA) or protein. Therefore, gene downregulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a normal cell). In one example, a control is a relative amount of gene expression or protein expression in a biological sample taken from a subject who does not have an ovarian tumor, such as serous ovarian cancer.

Endothelial cell: Cells that line the interior surface of blood vessels, forming an interface between circulating blood in the lumen and the rest of the vessel wall. For example, endothelial cells line the entire circulatory system. Further, both blood and lymphatic capillaries are composed of a single layer of endothelial cells.

Epithelial cell: Cells that line the interior surface of the lungs, the gastrointestinal tract, the reproductive and urinary tracts, and make up the exocrine and endocrine glands. Functions of epithelial cells include secretion, absorption, protection, transcellular transport, sensation detection, and selective permeability. Endothelium (the inner lining of blood vessels) is a specialized form of epithelium.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced. In an example, gene expression can be monitored to diagnosis and/or prognosis a subject with an ovarian tumor, such as predict a subject's survival time with advanced stage ovarian cancer.

The expression of a nucleic acid molecule can be altered relative to a normal (wild type) nucleic acid molecule. Alterations in gene expression, such as differential expression, include but are not limited to: (1) overexpression; (2) underexpression; or (3) suppression of expression. Alternations in the expression of a nucleic acid molecule can be associated with, and in fact cause, a change in expression of the corresponding protein.

Protein expression can also be altered in some manner to be different from the expression of the protein in a normal (wild type) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard. Controls or standards for comparison to a sample, for the determination of differential expression, include samples believed to be normal (in that they are not altered for the desired characteristic, for example a sample from a subject who does not have cancer, such as ovarian cancer) as well as laboratory values (e.g., range of values), even though possibly arbitrarily set, keeping in mind that such values can vary from laboratory to laboratory.

Laboratory standards and values can be set based on a known or determined population value and can be supplied in the format of a graph or table that permits comparison of measured, experimentally determined values.

Gene expression profile (or fingerprint): Differential or altered gene expression can be detected by changes in the detectable amount of gene expression (such as cDNA or mRNA) or by changes in the detectable amount of proteins expressed by those genes. A distinct or identifiable pattern of gene expression, for instance a pattern of high and low expression of a defined set of genes or gene-indicative nucleic acids such as ESTs; in some examples, as few as one or two genes provides a profile, but more genes can be used in a profile, for example at least 3, at least 4, at least 5, at least 6, at least 10, at least 20, at least 25, at least 30, at least 50, at least 80, at least 100, at least 190 or more of those listed in Tables 1 and 2. A gene expression profile (also referred to as a fingerprint) can be linked to a tissue or cell type (such as ovarian tumor cell), to a particular stage of normal tissue growth or disease progression (such as advanced ovarian cancer), or to any other distinct or identifiable condition that influences gene expression in a predictable way. Gene expression profiles can include relative as well as absolute expression levels of specific genes, and can be viewed in the context of a test sample compared to a baseline or control sample profile (such as a sample from a subject who does not have an ovarian tumor). In one example, a gene expression profile in a subject is read on an array (such as a nucleic acid or protein array). For example, a gene expression profile can be performed using a commercially available array such as a Human Genome U133 2.0 Plus Microarray from AFFYMETRIX® (Santa Clara, Calif.).

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Detects Sequences that Share at Least 80% Identity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share at Least 60% Identity)

| | |
|---|---|
| Hybridization: | 6x SSC at RT to 55° C. for 16-20 hours |
| Wash at least twice: | 2x-3x SSC at RT to 55° C. for 20-30 minutes each. |

Inhibitor: Any chemical compound, nucleic acid molecule or peptide (such as an antibody), specific for a nucleic acid molecule or gene product that can reduce activity of the gene product or directly interfere with expression of a gene. An inhibitor of the disclosure, for example, can inhibit the activity of a protein that is encoded by the gene (such as those listed in Table 1 or 2) either directly or indirectly. Direct inhibition can be accomplished, for example, by binding to a protein and thereby preventing the protein from binding an intended target, such as a receptor. Indirect inhibition can be accomplished, for example, by binding to a protein's intended target, such as a receptor or binding partner, thereby blocking or reducing activity of the protein. Furthermore, an inhibitor of the disclosure can inhibit a gene by reducing or inhibiting expression of the gene, inter alia by interfering with gene expression (transcription, processing, translation, post-translational modification), for example, by interfering with the gene's mRNA and blocking translation of the gene product or by post-translational modification of a gene product, or by causing changes in intracellular localization. In an example, ovarian survival factor-associated molecule is inhibited by use of a specific small interfering RNA (siRNA) or shRNA.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins. For example, an isolated cell, is a serous papillary ovarian cancer cell that is substantially separated from other ovarian cell subtypes, such as endometrioid, clear cell or mucinous subtypes.

Label: An agent capable of detection, for example by ELISA, spectrophotometry, flow cytometry, or microscopy. For example, a label can be attached to a nucleic acid molecule or protein (such as those listed in Table 1 or 2), thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998). In a particular example, a label is conjugated to a binding agent that specifically binds to one or more of the ovarian survival factor-associated molecules disclosed in Tables 1 and 2 to allow for the presence of a tumor in a subject.

Malignant: Cells that have the properties of anaplasia invasion and metastasis.

Mammal: This term includes both human and non-human mammals. Examples of mammals include, but are not limited to: humans, pigs, cows, goats, cats, dogs, rabbits and mice.

Matrix metallopeptidase 13 (MMP13): Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. Most MMPs are secreted as inactive proproteins which are activated when cleaved by extracellular proteinases. The protein encoded by this gene cleaves type II collagen more efficiently than types I and III. The gene is part of a cluster of MMP genes which localize to chromosome 11q22.3. In particular examples, expression of MMP13 is altered in an ovarian tumor. The term MMP13 includes any MMP13 gene, cDNA, mRNA, or protein from any organism and that is MMP13 and whose expression is increased in an ovarian tumor.

Nucleic acid and protein sequences for MMP13 are publicly available. For example, GENBANK® Accession Nos.: NM_{—}174389, BC125320, and NM_{—}002427 disclose MMP13 nucleic acid sequences, and GENBANK® Accession Nos.: AAH74808, AAI25321, and AAM51172 disclose MMP13 protein sequences, all of which are incorporated by reference as provided by GENBANK® on Apr. 13, 2007.

In one example, MMP13 includes a full-length wild-type (or native) sequence, as well as MMP13 allelic variants, fragments, homologs or fusion sequences that retain the ability to be overexpressed in an ovarian tumor and/or modulate an ovarian tumor activity, such as vascular growth. In certain examples, MMP13 has at least 80% sequence identity, for example at least 85%, 90%, 95%, or 98% sequence identity to MMP13. In other examples, MMP13 has a sequence that hybridizes to AFFYMETRIX® Probe ID No. 205959_at and retains MMP13 activity (such as the capability to be overexpressed in an ovarian tumor and/or modulate tumor and/or vascular growth).

Microfibril-associated glycoprotein 2 (MAGP2): MAGP2 induces adhesion in a number of different cell types via the αᵥβ₃ integrin receptor (Gibson et al., J. Biol. Chem. 271:1096-1103, 1999). MAGP-2 interacts with fibrillin-1 and -2, as well as fibulin-1 (another component of elastic fibers). In particular examples, expression of MAGP2 is altered, such as increased, in an ovarian tumor. The term MAGP2 includes any MAGP2 gene, cDNA, mRNA, or protein from any organism and that is MAGP2 and whose expression is increased in an ovarian tumor.

Nucleic acid and protein sequences for MAGP2 are publicly available. For example, GENBANK® Accession Nos.: NM_{—}174386, AF084918, and NM_{—}003480 disclose MAGP2 nucleic acid sequences, and GENBANK® Accession Nos.: NP_{—}003471, NP_{—}776811, and AAH05901 disclose MAGP2 protein sequences, all of which are incorporated by reference as provided by GENBANK® on Apr. 13, 2007.

In one example, MAGP2 includes a full-length wild-type (or native) sequence, as well as MAGP2 allelic variants, fragments, homologs or fusion sequences that retain the ability to be expressed in an ovarian tumor and/or modulate an ovarian tumor activity, such as vascular growth. In certain examples, MAGP2 has at least 80% sequence identity, for example at least 85%, 90%, 95%, or 98% sequence identity to MAGP2. In other examples, MAGP2 has a sequence that hybridizes to AFFYMETRIX® Probe ID No. 209758_s_at and retains MAGP2 activity (such as the capability to be overexpressed in an ovarian tumor and/or modulate tumor and/or vascular growth).

Neoplasm: Abnormal growth of cells.

Normal Cell Non-tumor cell, non-malignant, uninfected cell.

Nucleic acid array: An arrangement of nucleic acids (such as DNA or RNA) in assigned locations on a matrix, such as that found in cDNA arrays, or oligonucleotide arrays.

Nucleic acid molecules representing genes: Any nucleic acid, for example DNA (intron or exon or both), cDNA, or RNA (such as mRNA), of any length suitable for use as a probe or other indicator molecule, and that is informative about the corresponding gene, such as those listed in Table 1 or 2.

Nucleic acid molecules: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, a nucleic acid molecule can be circular or linear.

The disclosure includes isolated nucleic acid molecules that include specified lengths of an ovarian survival factor-associated molecule nucleotide sequence, such as those genes listed in Tables 1 and 2. Such molecules can include at least 10, at least 15, at least 20, at least 25, at least 30, at least 35, at least 40, at least 45 or at least 50 consecutive nucleotides of these sequences or more, and can be obtained from any region of a ovarian survival factor-associated molecule.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides. In one example, an oligonucleotide is a short sequence of nucleotides of at least one of the disclosed ovarian survival factor-associated molecules listed in Table 1 or 2.

Oligonucleotide probe: A short sequence of nucleotides, such as at least 8, at least 10, at least 15, at least 20, at least 21, at least 25, or at least 30 nucleotides in length, used to detect the presence of a complementary sequence by molecular hybridization. In particular examples, oligonucleotide probes include a label that permits detection of oligonucleotide probe:target sequence hybridization complexes. In one example, an oligonucleotide probe is a short sequence of nucleotides used to detect the presence of at least one of the disclosed ovarian survival factor-associated molecules listed in Table 1 or 2.

Ovarian tumor: A malignant ovarian neoplasm (an abnormal growth located on the ovaries including ovarian carcinoma, papillary serous cystadenocarcinoma, mucinous cystadenocarcinoma, endometrioid tumors, celioblastoma, clear cell carcinoma, unclassified carcinoma, granulosa-thecal cell tumors, Sertoli-Leydig cell tumors, dysgerminoma, and malignant teratoma. The most common type of ovarian tumor is papillary serous carcinoma.

Surgery is a treatment for an ovarian tumor and is frequently necessary for diagnosis. The type of surgery depends upon how widespread the tumor is when diagnosed (the tumor stage), as well as the type and grade of tumor. The surgeon may remove one (unilateral oophorectomy) or both ovaries (bilateral oophorectomy), the fallopian tubes (salpingectomy), and the uterus (hysterectomy). For some very early tumors (stage 1, low grade or low-risk disease), only the involved ovary and fallopian tube will be removed (called a “unilateral salpingo-oophorectomy,” USO), especially in young females who wish to preserve their fertility. In advanced disease as much tumor as possible is removed (debulking surgery). In cases where this type of surgery is successful, the prognosis is improved compared to subjects where large tumor masses (more than 1 cm in diameter) are left behind.

Chemotherapy is often used after surgery to treat any residual disease. Systemic chemotherapy often includes a platinum derivative with a taxane. Chemotherapy is also used to treat subjects who have a recurrence.

Ovarian survival factor-associated (or related) molecule: A molecule whose expression is altered in an ovarian tumor cell. Such molecules include, for instance, nucleic acid sequences (such as DNA, cDNA, or mRNAs) and proteins. Specific genes include those listed in Tables 1 and 2, as well as fragments of the full-length genes, cDNAs, or mRNAs (and proteins encoded thereby) whose expression is altered (such as upregulated or downregulated) in response to an ovarian tumor, including ovarian cancer. Thus, the presence or absence of the respective ovarian survival factor-associated molecules can be used to diagnose and/or determine the prognosis of an ovarian tumor in a subject as well as to treat a subject with an ovarian tumor, such as ovarian cancer.

In an example, an ovarian survival factor-associated molecule is any molecule listed in Tables 1 and 2. Specific examples of ovarian survival factor-associated molecules that are upregulated in a subject with a poor prognosis include MAGP2, Protein tyrosine phosphatase receptor D (PTPRD), KLB, Twist homologue 1 (TWIST1) and MMP13.

Ovarian survival factor-associated molecules can be involved in or influenced by cancer in different ways, including causative (in that a change in a ovarian survival factor-associated molecule leads to development of or progression of ovarian cancer) or resultive (in that development of or progression of ovarian cancer causes or results in a change in the ovarian survival factor-associated molecule).

Pharmaceutically acceptable carriers: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic agents, such as one or more compositions that include a binding agent that specifically binds to at least one of the disclosed ovarian survival factor-associated molecules.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

Protein tyrosine phosphatase receptor D (PTPRD): A protein with protein tyrosine phosphatase activity that can regulate receptor tyrosine kinases. In particular examples, expression of PTPRD is increased in an ovarian tumor. The term PTPRD includes any PTPRD gene, cDNA, mRNA, or protein from any organism and that is PTPRD and is expressed in an ovarian tumor.

Nucleic acid and protein sequences for PTPRD are publicly available. For example, GENBANK® Accession Nos.: BC106715, BC106714, and NM_{—}019140 disclose PTPRD nucleic acid sequences, and GENBANK® Accession Nos.: CAI25771, CAI25475, and AAI06716 disclose PTPRD protein sequences, all of which are incorporated by reference as provided by GENBANK® on Apr. 13, 2007.

In one example, PTPRD includes a full-length wild-type (or native) sequence, as well as PTPRD allelic variants, fragments, homologs or fusion sequences that retain the ability to be increased in an ovarian tumor and/or modulate an ovarian tumor activity, such as vascular growth. In certain examples, PTPRD has at least 80% sequence identity, for example at least 85%, 90%, 95%, or 98% sequence identity to PTPRD. In other examples, PTPRD has a sequence that hybridizes to AFFYMETRIX® Probe ID No. 214043_at and retains PTPRD activity (such as the capability to be increased in an ovarian tumor and/or modulate tumor and/or vascular growth).

Polymerase Chain Reaction (PCR): An in vitro amplification technique that increases the number of copies of a nucleic acid molecule (for example, a nucleic acid molecule in a sample or specimen). In an example, a biological sample collected from a subject is contacted with a pair of oligonucleotide primers, under conditions that allow for the hybridization of the primers to nucleic acid template in the sample (such as those listed in Table 1 or 2). The primers are extended under suitable conditions, dissociated from the template, and then re-annealed, extended, and dissociated to amplify the number of copies of the nucleic acid. The product of a PCR can be characterized by electrophoresis, restriction endonuclease cleavage patterns, oligonucleotide hybridization or ligation, and/or nucleic acid sequencing, using standard techniques or other standard techniques known in the art.

Primers: Short nucleic acid molecules, for instance DNA oligonucleotides 10-100 nucleotides in length, such as about 15, 20, 25, 30 or 50 nucleotides or more in length. Primers can be annealed to a complementary target DNA strand by nucleic acid hybridization to form a hybrid between the primer and the target DNA strand (such as those listed in Table 1 or 2). Primer pairs can be used for amplification of a nucleic acid sequence, such as by PCR or other nucleic acid amplification methods known in the art.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5., © 1991, Whitehead Institute for Biomedical Research, Cambridge, Mass.). One of ordinary skill in the art will appreciate that the specificity of a particular primer increases with its length. Thus, for example, a primer including 30 consecutive nucleotides of an ovarian survival factor-associated molecule will anneal to a target sequence, such as another homolog of the designated ovarian survival factor-associated protein, with a higher specificity than a corresponding primer of only 15 nucleotides. Thus, in order to obtain greater specificity, primers can be selected that include at least 20, at least 25, at least 30, at least 35, at least 40, at least 45, at least 50 or more consecutive nucleotides of an ovarian tumor survival factor-associated nucleotide sequence.

Prognosis: A prediction of the course of a disease, such as serous ovarian cancer. The prediction can include determining the likelihood of a subject to develop aggressive, recurrent disease, to survive a particular amount of time (e.g. determine the likelihood that a subject will survive 1, 2, 3 or 5 years), to respond to a particular therapy (e.g., chemotherapy), or combinations thereof.

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. Similarly, a purified oligonucleotide preparation is one in which the oligonucleotide is more pure than in an environment including a complex mixture of oligonucleotides.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

RNA interference (RNAi): A post-transcriptional gene silencing mechanism mediated by double-stranded RNA (dsRNA). Introduction of dsRNA into cells, such as by introduction of synthetic ds siRNAs or by vector systems that express ds shRNAs that are subsequently processed to siRNAs by cellular machinery, induces targeted degradation of RNA molecules with homologous sequences. RNAi compounds can be used to modulate transcription, for example, by silencing genes, such as ovarian survival factor-associated molecules listed in Table 1 or 2 (for example by targeting at least 20 contiguous nucleotides of MAGP2). In certain examples, an RNAi molecule is directed against a target, such as MAGP2, and is used to decrease expression of MAGP2 in an ovarian tumor.

Sample (or biological sample): A biological specimen containing genomic DNA, RNA (including mRNA), protein, or combinations thereof, obtained from a subject. Examples include, but are not limited to, peripheral blood, urine, saliva, tissue biopsy, surgical specimen, and autopsy material. In one example, a sample includes an ovarian cancer tissue biopsy, such as a homogenous tumor epithelial sample.

Sensitivity: A measurement of activity, such as biological activity, of a molecule or a collection of molecules in a given condition. In an example, sensitivity refers to the activity of an agent, such as a binding agent that preferentially binds to one or more ovarian survival factor-associated molecules (such as those listed in Table 1 or 2), to alter the growth, development or progression of a disease, such as ovarian cancer. In certain examples, sensitivity or responsiveness can be assessed using any endpoint indicating a benefit to the subject, including, without limitation, (1) inhibition, to some extent, of tumor growth, including slowing down and complete growth arrest; (2) reduction in the number of tumor cells; (3) reduction in tumor size; (4) inhibition (such as reduction, slowing down or complete stopping) of tumor cell infiltration into adjacent peripheral organs and/or tissues; (5) inhibition (such as reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs may use SEG. In addition, a manual alignment can be performed. Proteins with even greater similarity will show increasing percentage identities when assessed by this method, such as at least about 75%, 80%, 85%, 90%, 95%, 98%, or 99% sequence identity to a molecule listed in Table 1 or 2.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity to a molecule listed in Table 1 or 2. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to a molecule listed in Table 1 or 2 determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Short hairpin RNA (shRNA): A sequence of RNA that makes a hairpin turn that can be used to reduce or silence gene expression. shRNAs can be synthesized exogenously or can be transcribed from RNA polymerase III promoters in vivo (for example using a viral vector), thus permitting long-term gene silencing. shRNAs are processed into siRNAs by cellular machinery. In a particular example, an shRNA molecule targets a ovarian survival factor-associated molecule listed in Table 1 or Table 2 that are increased in an ovarian tumor (such as MAGP2), thereby decreasing expression of the molecule. Viral vectors, such as lentiviral and adenoviral vectors, permit delivery and stable expression of shRNA in a mammalian cell that include both the sequence homologous to ovarian survival factor-associated molecule and the complimentary strand with an intervening non-complimentary linkage segment.

Short interfering RNA (siRNA): A double stranded nucleic acid molecule capable of RNA interference or “RNAi.” (See, for example, Bass Nature 411:428-9, 2001; Elbashir et al., Nature 411:494-8, 2001; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.) As used herein, siRNA molecules need not be limited to those molecules containing only RNA, but further encompasses chemically modified nucleotides and non-nucleotides having RNAi capacity or activity. In an example, an siRNA molecule is one that reduces or inhibits the biological activity or expression of one or more ovarian survival factor-associated molecules disclosed in Tables 1 or 2 that are upregulated in ovarian tumor epithelial cells, such as MAGP2, PTPRD, KLB, TWIST1, and MMP13. In some examples, commercially available kits, such as siRNA molecule synthesizing kits from PROMEGA® (Madison, Wis.) or AMBION® (Austin, Tex.) may be used to synthesize siRNA molecules. In other examples, siRNAs are obtained from commercial sources, such as from QIAGEN® Inc (Germantown, Md.), INVITROGEN® (Carlsbad, Calif.), AMBION (Austin, Tex.), DHARMACON® (Lafayette, Colo.), SIGMA-ALDRICH® (Saint Louis, Mo.) or OPENBIOSYSTEMS® (Huntsville, Ala.). In a particular example, a MAGP2 siRNA molecule has the following sequence: 5′-ACCGGTTAAACAATGCATTCAT-3′ (sense; SEQ ID NO: 1) and 5′-ATGAATGCATTGTTTAACCGGC-3′ (antisense; SEQ ID NO: 2).

Specific Binding Agent: An agent that binds substantially or preferentially only to a defined target such as a protein, enzyme, polysaccharide, oligonucleotide, DNA, RNA, recombinant vector or a small molecule. In an example, a “specific binding agent” is capable of binding to at least one of the disclosed ovarian survival factor-associated molecules (such as those listed in Table 1 or 2). Thus, a RNA-specific binding agent binds substantially only to the defined RNA, or to a specific region within the RNA. For example, a “specific binding agent” includes a siRNA that bind substantially to a specified RNA.

A protein-specific binding agent binds substantially only the defined protein, or to a specific region within the protein. For example, a “specific binding agent” includes antibodies and other agents that bind substantially to a specified polypeptide. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Stanniocalcin 2 (STC2): This gene encodes a secreted, homodimeric glycoprotein that is expressed in a variety of tissues and has autocrine or paracrine functions. The encoded protein has 10 of its 15 cysteine residues conserved among stanniocalcin family members and is phosphorylated by casein kinase 2 exclusively on its serine residues. Its C-terminus contains a cluster of histidine residues, which may interact with metal ions. The protein may play a role in the regulation of renal and intestinal calcium and phosphate transport, cell metabolism, or cellular calcium/phosphate homeostasis. Constitutive overexpression of human stanniocalcin 2 in mice resulted in pre- and postnatal growth restriction, reduced bone and skeletal muscle growth, and organomegaly. Expression of this gene is induced by estrogen and altered in some breast cancers. In particular examples, expression of STC2 is increased in an ovarian tumor. The term STC2 includes any STC2 gene, cDNA, mRNA, or protein from any organism and that is STC2 and is expressed in an ovarian tumor.

Nucleic acid and protein sequences for STC2 are publicly available. For example, GENBANK® Accession Nos.: NM_{—}003714, NM_{—}011491, and NM_{—}022230 disclose STC2 nucleic acid sequences, and GENBANK® Accession Nos.: CAG46624, NP_{—}035621, and NP_{—}071566 disclose STC2 protein sequences, all of which are incorporated by reference as provided by GENBANK® on Apr. 13, 2007.

In one example, STC2 includes a full-length wild-type (or native) sequence, as well as STC2 allelic variants, fragments, homologs or fusion sequences that retain the ability to be upregulated in an ovarian tumor and/or modulate an ovarian tumor activity, such as vascular growth. In certain examples, STC2 has at least 80% sequence identity, for example at least 85%, 90%, 95%, or 98% sequence identity to STC2. In other examples, STC2 has a sequence that hybridizes to AFFYMETRIX® Probe ID No. 203439_s_at and retains STC2 activity (such as the capability to be increased in expression in an ovarian tumor and/or modulate tumor and/or vascular growth).

Subject: Living multi-cellular vertebrate organisms, a category that includes human and non-human mammals.

Target sequence: A sequence of nucleotides located in a particular region in the human genome that corresponds to a desired sequence, such as an ovarian survival factor-associated sequence. Target sequences can encode target proteins. The target can be for instance a coding sequence; it can also be the non-coding strand that corresponds to a coding sequence. Examples of target sequences include those sequences associated with ovarian survival factor-associated cells, such as any of those listed in Table 1 or 2.

Therapeutically Effective Amount: An amount of a composition that alone, or together with an additional therapeutic agent(s) (for example a chemotherapeutic agent), induces the desired response (e.g., treatment of a tumor). The preparations disclosed herein are administered in therapeutically effective amounts. In one example, a desired response is to decrease tumor size or metastasis in a subject to whom the therapy is administered. Tumor metastasis does not need to be completely eliminated for the composition to be effective. For example, a composition can decrease metastasis by a desired amount, for example by at least 20%, at least 50%, at least 60%, at least 70%, at least 80%, at least 90%, at least 95%, at least 98%, or even at least 100% (elimination of the tumor), as compared to metastasis in the absence of the composition.

In particular examples, it is an amount of the therapeutic agent conjugated to a specific binding agent effective to decrease a number of ovarian cancer cells, such as in a subject to whom it is administered, for example a subject having one or more carcinomas. The cancer cells do not need to be completely eliminated for the composition to be effective. For example, a composition can decrease the number of cancer cells by a desired amount, for example by at least 20%, at least 50%, at least 60%, at least 70%, at least 80%, at least 90%, at least 95%, at least 98%, or even at least 100% (elimination of detectable cancer cells), as compared to the number of cancer cells in the absence of the composition.

In other examples, it is an amount of a specific binding agent for one or more of the disclosed ovarian survival factor-associated molecules capable of reducing angiogenesis by least 20%, at least 50%, at least 60%, at least 70%, at least 80%, at least 90%, at least 95%, at least 98%, or even at least 100% (elimination of detectable angiogenesis) by the specific binding agent, or both, effective to decrease the metastasis of a tumor.

A therapeutically effective amount of a specific binding agent for at least one of the disclosed ovarian survival factor-associated molecules, or cancer cells lysed by a therapeutic molecule conjugated to the agent, can be administered in a single dose, or in several doses, for example daily, during a course of treatment. However, the therapeutically effective amount can depend on the subject being treated, the severity and type of the condition being treated, and the manner of administration. For example, a therapeutically effective amount of such agent can vary from about 1 μg-10 mg per 70 kg body weight if administered intravenously and about 10 μg-100 mg per 70 kg body weight if administered intratumorally.

Tissue: A plurality of functionally related cells. A tissue can be a suspension, a semi-solid, or solid. Tissue includes cells collected from a subject, such as the ovaries.

Treating a disease: “Treatment” refers to a therapeutic intervention that ameliorates a sign or symptom of a disease or pathological condition, such as a sign or symptom of ovarian cancer. Treatment can also induce remission or cure of a condition, such as ovarian cancer. In particular examples, treatment includes preventing a disease, for example by inhibiting the full development of a disease or metastasis of a tumor. Prevention of a disease does not require a total absence of disease. For example, a decrease of at least 50% can be sufficient.

Tumor: All neoplastic cell growth and proliferation, whether malignant or benign, and all pre-cancerous and cancerous cells and tissues. In an example, a tumor is an ovarian tumor.

Tumor-necrosis factor, alpha-induced protein 6 (TNFAIP6): A protein capable of regulating the expression of various molecules involved in the control of inflammation. In particular examples, expression of TNFAIP6 is increased in an ovarian tumor. The term TNFAIP6 includes any TNFAIP6 gene, cDNA, mRNA, or protein from any organism and that is TNFAIP6 and is increased in ovarian tumor.

Nucleic acid and protein sequences for TNFAIP6 are publicly available. For example, GENBANK® Accession Nos.: NM_{—}007115, BC021155 and NM_{—}009398 disclose TNFAIP6 nucleic acid sequences, and GENBANK® Accession Nos.: AAH21155, NP_{—}009046 and NP_{—}033424 disclose TNFAIP6 protein sequences, all of which are incorporated by reference as provided by GENBANK® on Apr. 13, 2007.

In one example, TNFAIP6 includes a full-length wild-type (or native) sequence, as well as TNFAIP6 allelic variants, fragments, homologs or fusion sequences that retain the ability to be upregulated in an ovarian tumor and/or modulate ovarian tumor activity, such as vascular growth. In certain examples, TNFAIP6 has at least 80% sequence identity, for example at least 85%, 90%, 95%, or 98% sequence identity to TNFAIP6. In other examples, TNFAIP6 has a sequence that hybridizes to AFFYMETRIX® Probe ID No. 206026_s_at and retains TNFAIP6 activity (such as the capability to be increased in an ovarian tumor and/or modulate tumor and/or vascular growth).

Twist homologue 1 (TWIST1): Overexpression of TWIST1 has been reported to participate in destabilizing the genome, thus promoting chromosomal instability. For example, TWIST1 is capable of inhibiting chrondrogenesis. TWIST1 protein is involved in the regulation of tumor necrosis factor alpha production by antiinflammatory factors and pathways. In particular examples, expression of TWIST1 is increased in an ovarian tumor. The term TWIST1 includes any TWIST1 gene, cDNA, mRNA, or protein from any organism and that is TWIST1 and is expressed in ovarian tumor.

Nucleic acid and protein sequences for TWIST1 are publicly available. For example, GENBANK® Accession Nos.: NM_{—}000474, NM_{—}053530 and XM_{—}001076553 and disclose TWIST1 nucleic acid sequences, and GENBANK® Accession Nos.: NP_{—}000465 and ABM87769 disclose TWIST1 protein sequences, all of which are incorporated by reference as provided by GENBANK® on Apr. 13, 2007.

In one example, TWIST1 includes a full-length wild-type (or native) sequence, as well as TWIST1 allelic variants, fragments, homologs or fusion sequences that retain the ability to be upregulated in an ovarian tumor and/or modulate ovarian tumor activity, such as vascular growth. In certain examples, TWIST1 has at least 80% sequence identity, for example at least 85%, 90%, 95%, or 98% sequence identity to TWIST1. In other examples, TNFAIP6 has a sequence that hybridizes to AFFYMETRIX® Probe ID No. 206026_s_at and retains TWIST1 activity (such as the capability to be increased in an ovarian tumor and/or modulate tumor activity and/or vascular growth).

Under conditions sufficient for: A phrase that is used to describe any environment that permits the desired activity. In one example, includes administering a test agent to an ovarian cancer cell or a subject sufficient to allow the desired activity. In particular examples, the desired activity is altering the activity (such as the expression) of an ovarian survival factor-associated molecule.

Unit dose: A physically discrete unit containing a predetermined quantity of an active material calculated to individually or collectively produce a desired effect, such as a therapeutic effect. A single unit dose or a plurality of unit doses can be used to provide the desired effect, such as treatment of an ovarian tumor, for example a metastatic tumor. In one example, a unit dose includes a desired amount of an agent that decreases or inhibits angiogenesis. In a particular example, a unit dose includes a desired amount of an agent that decreases or inhibits an ovarian survival factor-associated molecule that is upregulated in advanced ovarian papillary serous cancer.

Upregulated or activation: When used in reference to the expression of a nucleic acid molecule, such as a gene, refers to any process which results in an increase in production of a gene product. A gene product can be RNA (such as mRNA, rRNA, tRNA, and structural RNA) or protein. Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product (such as those listed in Tables 1 and 2) increases by at least 2-fold, for example at least 3-fold or at least 4-fold, as compared to a control (such an amount of gene expression in a normal cell). In one example, a control is a relative amount of gene expression in a biological sample, such as in an ovarian tissue biopsy obtained from a subject that does not have ovarian cancer.

Additional terms commonly used in molecular genetics can be found in Benjamin Lewin, Genes V published by Oxford University Press, 1994 (ISBN 0-19-854287-9); Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### Methods of Diagnosing and Prognosing an Ovarian Tumor

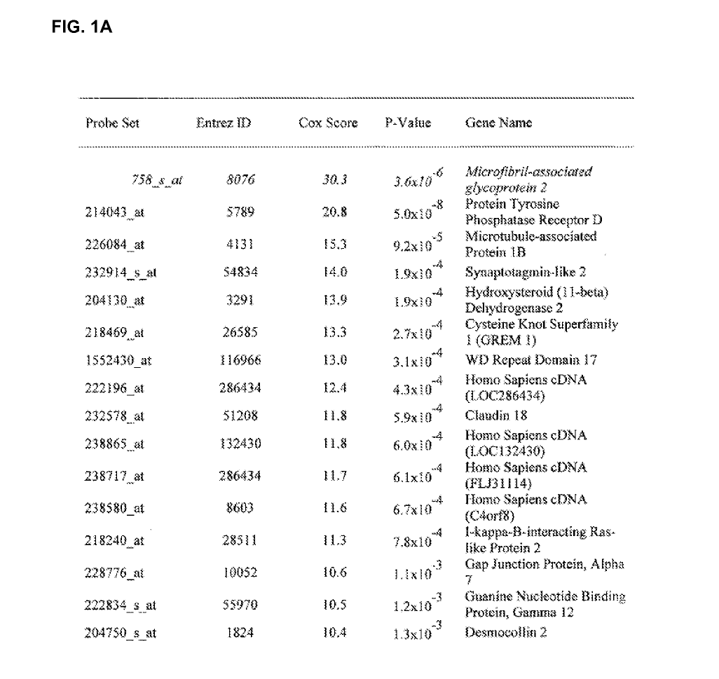

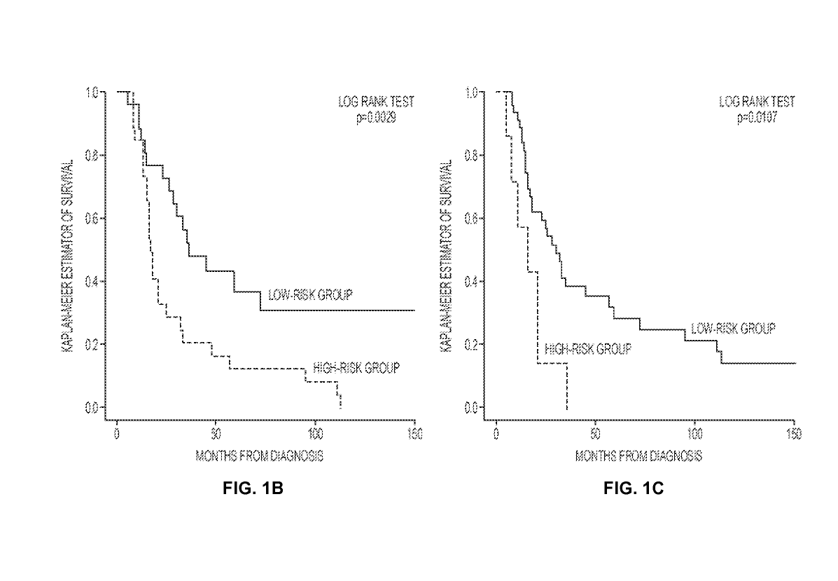

Methods are disclosed for diagnosing and prognosing an ovarian tumor, such as papillary serous ovarian cancer, in a subject. In one example, the methods include detecting expression of at least one (such as at least 3, at least 4, at least 5, at least 6, at least 10, at least 20, at least 25, at least 30, at least 50, at least 80, at least 100, at least 190 or more) ovarian survival factor-associated molecule listed in Table 1 or Table 2 in a sample obtained from the subject with the ovarian tumor. In some examples, the ovarian survival factor-associated molecule can include, consist essentially of, or consist of those listed in Table 1, Table 2, FIG. 1B, or combinations thereof. “Consists essentially of” in this context indicates that the expression of additional molecules can be evaluated (such as a control), but that these molecules do not include more than six other ovarian survival factor-associated molecules. Thus, in one example, the expression of a control, such as a housekeeping protein or rRNA can be assessed (such as 18S RNA, beta-microglobulin, GAPDH, and/or 18S rRNA). In some examples, “consist essentially of” indicates that no more than 5 other molecules are evaluated, such as no more than 4, 3, 2, or 1 other molecules. In this context “consist of” indicates that only the expression of the stated molecules are evaluated; the expression of additional molecules is not evaluated.

The methods also can include comparing expression of the at least one ovarian survival factor-associated molecule in the sample obtained from the subject with the ovarian tumor to a control, wherein an alteration in the expression of the at least one ovarian survival factor-associated molecule relative to the control indicates that the subject has a decreased chance of survival. For example, an increase in the expression of MAGP2, PTPRD, MMP13, STC2, CCRL1 or KLB relative to a normal control sample or reference value (or range of values) indicates a poor prognosis, such as a decreased chance of survival. In an example, a decreased chance of survival includes a survival time of equal to or less than 50 months, such as 40 months, 30 months, 20 months, 12 months, 6 months or 3 months from time of diagnosis. Conversely, a decrease in expression of an ovarian survival factor-associated molecule or expression levels similar to those in control levels indicates a better prognosis, such as an increased chance of survival (e.g., survival time of at least 50 months from time of diagnosis, such as 60 months, 80 months, 100 months, 120 months or 150 months from time of diagnosis). For example, the level of the ovarian survival factor-associated molecules detected can be compared to a control or reference value, such as a value that represents a level of an ovarian survival factor-associated molecule expected if a subject does not have an ovarian tumor. In one example, the ovarian survival factor-associated molecules detected in a tumor sample are compared to the level of such molecules detected in a sample obtained from a subject that does not have an ovarian tumor. In certain examples, detection of at least a 2-fold, such as at least 3-fold, at least 4-fold, at least 6-fold or at least 10-fold increase in the relative amount of the ovarian survival factor-associated molecules in a tumor sample, as compared to the relative amount of such molecules in a control, indicates that the subject has tumor, such as a grade 3 ovarian tumor, has a poor prognosis (e.g., survival time of less than 50 months from time of diagnosis, such as 40 months, 30 months, 20 months, 12 months, 6 months or 3 months from time of diagnosis), or combinations thereof. In some examples, detection of statistically similar relative amounts (or decreased amounts) of ovarian survival factor-associated molecules observed in a non-tumor sample, as compared to the relative amount of such molecules in a control sample, indicates that that subject does not have a tumor, such as a grade 3 ovarian tumor, has a good prognosis (survival time of at least 50 months from time of diagnosis, such as 60 months, 80 months, 100 months, 120 months or 150 months from time of diagnosis), or combinations thereof.

Alterations in the expression can be measured at the nucleic acid level (such as by real time quantitative polymerase chain reaction or microarray analysis) or at the protein level (such as by Western blot analysis). In a particular example, a method of diagnosing and prognosing ovarian papillary serous cancer is provided.

In some examples, the method includes determining the metastatic potential of an ovarian tumor in a subject by detecting expression of at least one ovarian survival factor-associated molecule in a sample obtained from a subject with an ovarian tumor. The at least one ovarian survival factor-associated molecule is involved in promoting angiogenesis, such as cell proliferation, cell motility or tube formation. Examples of such molecules include TWIST1, TWIST2, MAGP2, CCRL, ENPEP, TNFAIP6, PPIBP1, DSC2, SFTPD, FGF18, NEDD9, FGFR2, FGB, FLRT3, ANGPT2, MMP12, MMP13, C12orf9, PCDH10, STC2, and ZAK. The method can further include comparing expression of the at least one ovarian survival factor-associated molecule in the sample obtained from the subject with the ovarian tumor to a control (such as a normal sample or range of values expected from a sample not containing cancer cells). An alteration in the expression relative to the control, such as an increase, of the at least one ovarian survival factor-associated molecule involved in promoting angiogenesis indicates that the subject has an ovarian tumor with increased metastatic potential.

Metastasis is a major complication in the pathogenesis of tumors, such as ovarian cancer, and is typically indicative of poor prognosis. It is also known that angiogenesis is a factor in the progression of solid tumors and metastases, including ovarian cancer. The formation of the vascular stroma plays a role in the pathophysiology of malignancy. For instance, in the absence of vascular support tumors may become necrotic, or even apoptotic. In contrast, the onset of angiogenesis marks a phase of rapid proliferation, local invasion, and ultimately metastasis.

Without wishing to be bound to a particular theory, it is proposed that increased expression of the disclosed ovarian survival factor-associated molecules associated with angiogenesis, such as molecules involved in cell proliferation, cell motility, cell adhesion or tube formation, is related to enhanced ovarian tumor cell metastasis. Conversely, a decreased expression of the disclosed ovarian survivial factor-associated molecules can be correlated with a better or more favorable prognosis, such as an increased chance of survival. Thus, methods of diagnosing or prognosing an ovarian tumor that expresses at least one pro-angiogenic ovarian survival factor-associated molecule, are disclosed. In some examples, such methods can be used to identify those subjects that will benefit from the disclosed treatment methods. For example, such diagnostic methods can be performed prior to the subject undergoing the treatments described above. In other examples, these methods are utilized to predict the metastatic potential of the ovarian cancer, subject survival, or combinations thereof.

In an example, the method includes detecting expression of at least one pro-angiogenic ovarian survival factor-associated molecule listed in Tables 1 and 2 in a sample from the subject exhibiting one or more symptoms associated with ovarian cancer. In a particular example, the specific pro-angiogenic ovarian survival factor-associated molecule is detected in a biological sample. For example, the biological sample can be a tumor biopsy, such as a biopsy sample containing epithelial cells. In another example, the pro-angiogenic ovarian survival factor-associated molecule is detected in a serum sample. For example, the ovarian survival factor-associated molecule can be detected in a serum sample if the specific molecule is known to be secreted or located on a cell surface susceptible to enzymatic cleavage.

In one example, detection of at least one ovarian survival factor-associated molecule listed in any of Tables 1 and 2 (such as pro-angiogenic ovarian survival factor-associated molecules) in a biological sample from the subject is used to diagnose or prognose an ovarian tumor. Methods of detecting such molecules in a sample are known in the art and are routine. In some examples, the relative amount of pro-angiogenic ovarian survival factor-associated molecules present is determined, for example by quantitating the expression level of such molecules. For example, the relative or absolute quantity of the at least one ovarian survival factor-associated molecule in a sample can be determined.

The activity such as the expression level of the disclosed ovarian survival factor-associated molecules in a sample obtained from a subject is compared to a control (such as a normal sample or range of values expected from a sample not containing cancer cells). An increase in expression of the pro-angiogenic ovarian survival factor-associated molecules above background or control levels indicates the presence of an ovarian tumor, the ovarian tumor is metastatic, the ovarian tumor is likely to become metastatic, or a combination thereof. Conversely, a decrease in expression of the pro-angiogenic ovarian survival factor-associated molecules or expression levels similar to those in control levels indicates a better prognosis, such as an increased chance of survival (e.g., survival time of at least 50 months from time of diagnosis, such as 60 months, 80 months, 100 months, 120 months or 150 months from time of diagnosis. For example, the level of the pro-angiogenic ovarian survival factor-associated molecules detected can be compared to a control or reference value, such as a value that represents a level of pro-angiogenic ovarian survival factor-associated molecules expected if an ovarian tumor is or is not metastatic. In one example, the pro-angiogenic ovarian survival factor-associated molecules detected in a tumor sample are compared to the level of such molecules detected in a sample obtained from a subject that does not have an ovarian tumor or has a non-metastatic ovarian tumor. In certain examples, detection of at least a 2-fold, such as at least 3-fold, at least 4-fold, at least 6-fold or at least 10-fold increase in the relative amount of the pro-angiogenic ovarian survival factor-associated molecules in a tumor sample, as compared to the relative amount of such molecules in a control, indicates that the subject has tumor with metastatic potential, has a tumor that has metastasized, has a poor prognosis (e.g., survival time of less than 50 months from time of diagnosis, such as 40 months, 30 months, 20 months, 12 months, 6 months or 3 months from time of diagnosis), or combinations thereof. In some examples, detection of statistically similar relative amounts (or decreased amounts) of pro-angiogenic ovarian survival factor-associated molecules observed in a tumor sample, as compared to the relative amount of such molecules in a control sample, indicates that that subject does not have a tumor with metastatic potential, does not have a tumor that has metastasized, has a good prognosis (survival time of at least 50 months from time of diagnosis, such as 60 months, 80 months, 100 months, 120 months or 150 months from time of diagnosis), or combinations thereof.

In a specific example, the method includes detecting and comparing the nucleic acid expression levels of the pro-angiogenic ovarian survival factor-associated molecules such as DNA, cDNA, or mRNAs. In a specific example, the method includes detecting and comparing the mRNA expression levels of the pro-angiogenic ovarian survival factor-associated molecules. For example, such expression is measured by real time quantitative polymerase chain reaction or microarray analysis. In a particular example, the disclosed gene expression profile is utilized to diagnosis and/or prognosis an ovarian tumor.

### Detection of Ovarian Survival Factor-Associated Nucleic Acids

In one example, one or more ovarian survival factor-associated molecules can be detected by polymerase chain reaction (PCR). The biological sample can be incubated with primers that permit the amplification of one or more of the disclosed ovarian survival factor-associated mRNAs, under conditions sufficient to permit amplification of such products.

In another example, the biological sample is incubated with probes that can bind to one or more of the disclosed ovarian survival factor-associated nucleic acid sequences (such as cDNA, genomic DNA, or RNA (such as mRNA)) under high stringency conditions. The resulting hybridization can then be detected using methods known in the art, such as by Northern blot analysis.

In an example, the isolated nucleic acid molecules or amplification products are incubated with the array including oligonucleotides complementary to the ovarian survival factor-associated molecules listed in Tables 1 or 2 for a time sufficient to allow hybridization between the isolated nucleic acid molecules and oligonucleotide probes, thereby forming isolated nucleic acid molecule:oligonucleotide complexes. The isolated nucleic acid molecule:oligonucleotide complexes are then analyzed to determine if expression of the isolated nucleic acid molecules is altered.

In particular examples, a therapeutic agent can be identified by applying the isolated nucleic acid molecules or amplification products to an array in which the isolated nucleic acid molecules are obtained from a biological sample including ovarian epithelial cancer cells following treatment with the one or more test agents. In such example, the array includes oligonucleotides complementary to all ovarian survival factor-associated genes listed in Table 1. In a particular example, the array is a commercially available array such as a U133 Plus 2.0 oligonucleotide array from AFFYMETRIX® (AFFYMETRIX®, Santa Clara, Calif.).

### Gene Expression Profile

The disclosed gene profile can be used in the diagnosis and prognosis of an ovarian tumor in a subject. In an example, the gene expression profile includes at least two of the ovarian survival factor-associated molecules listed in Table 1, Table 2, FIG. 1B, or combinations thereof, such as at least 5, at least 7, at least 10, at least 20, at least 30, at least 40, at least 50, at least 60, at least 70, at least 80, at least 90, at least 100, at least 150 or at least 175 molecules (for example, 2, 6, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 130, 140, 150, 170, 180, 190 or 200 of those listed). In some examples, the expression profile consists or consists essentially of the ovarian survival factor-associated molecules listed in Table 1, Table 2, FIG. 1B, or combinations thereof. In some examples, additional control molecules can be analyzed (e.g., 1-10 controls).

### Detecting Ovarian Survival Factor-Associated Proteins

As an alternative to analyzing the sample for the presence of nucleic acids, alterations in protein expression can be measured by methods known in the art, such as by Western blot analysis, immunoassay, mass spectrometry or a protein microarray. For example, the metastatic potential of an ovarian tumor can be determined by using a protein array that includes one or more capture agents, such as antibodies that are specific for the one or more disclosed ovarian survival factor-associated molecules that are related to angiogenesis, such as molecules that play a role in cell proliferation, cell motility, cell adhesion or tube formation.

In one example, the antibody that specifically binds an ovarian survival factor-associated molecule (such as those listed in Table 1 or 2) is directly labeled with a detectable label. In another example, each antibody that specifically binds an ovarian survival factor-associated molecule (the first antibody) is unlabeled and a second antibody or other molecule that can bind the human antibody that specifically binds the respective ovarian survival factor-associated molecule is labeled. As is well known to one of skill in the art, a second antibody is chosen that is able to specifically bind the specific species and class of the first antibody. For example, if the first antibody is a human IgG, then the secondary antibody can be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, Cy3, Cy5, fluorescein, fluorescein isothiocyanate, rhodamine, dichlorotriazinylamine fluorescein, dansyl chloride or phycoerythrin. A non-limiting exemplary luminescent material is luminol; a non-limiting exemplary magnetic agent is gadolinium, and non-limiting exemplary radioactive labels include ¹²⁵I, ¹³¹I, ³⁵S or ³H.

In an alternative example, ovarian survival factor-associated molecules can be assayed in a biological sample by a competition immunoassay utilizing ovarian survival factor-associated molecule standards labeled with a detectable substance and unlabeled antibody that specifically bind to the desired ovarian survival factor-associated molecule. In this assay, the biological sample (such as serum, tissue biopsy, or cells isolated from a tissue biopsy), the labeled ovarian survival factor-associated molecule standards and the antibody that specifically binds to ovarian survival factor-associated molecule are combined and the amount of labeled ovarian survival factor-associated molecule standard bound to the unlabeled antibody is determined. The amount of ovarian survival factor-associated molecule in the biological sample is inversely proportional to the amount of labeled ovarian survival factor-associated molecule standard bound to the antibody that specifically binds the ovarian survival factor-associated molecule.

### Methods of Treatment

It is shown herein that an ovarian tumor is associated with differential expression of ovarian survival factor-associated molecules. For example, the disclosed gene expression profile has identified ovarian survival factor-associated molecules. Based on these observations, methods of treatment to reduce or eliminate an ovarian tumor are disclosed by decreasing the expression of at least one of the ovarian survival factor-associated molecules from Tables 1 or 2. In a particular example, the subject is a human. In other certain examples, the subject is a veterinary subject. In an example, the ovarian tumor is advanced papillary serous ovarian cancer.

Methods are disclosed herein for treating an ovarian tumor, such as ovarian cancer. In one example, the method includes administering a therapeutically effective amount of a composition to a subject in which the composition includes an agent that decreases the biological activity (e.g., expression) of one or more of the ovarian survival factor-associated molecules listed in any of Tables 1 or 2. Such agents can alter the expression of nucleic acid sequences (such as DNA, cDNA, or mRNAs) and proteins. A decrease in the expression does not need to be 100% for the composition to be effective. For example, a composition can decrease the expression or biological activity by a desired amount, for example by at least 20%, at least 50%, at least 60%, at least 70%, at least 80%, at least 90%, at least 95%, at least 98%, or even at least 100% as compared to activity or expression in a control.

In particular examples, the agent is a specific binding agent that binds to and decreases the expression of one or more of the ovarian survival factor-associated molecules listed in Tables 1 or 2. Specific molecules include those listed in Tables 1 or 2 as well as fragments of the full-length molecules, cDNAs, or mRNAs (and proteins encoded thereby) whose expression is increased in response to an ovarian tumor, such as ovarian cancer. The agents can alter the activity of one or more of the ovarian survival factor-associated molecules listed in Tables 1 or 2 as well as other molecules involved in tumor progression in ovarian tumor cells themselves, epithelial cells, endothelial cells, fibroblasts, and/or immune cells. For example, an agent can decrease expression of one or more of the disclosed ovarian survival factor-associated molecules (such as one known to be secreted or associated with the cell surface) in epithelial tumor cells which then alters/modulates the behavior of other cells involved in tumor progression including endothelial cells, fibroblasts and immune cells.

In particular examples, the agent is an inhibitor such as a siRNA or an antibody to one of the disclosed ovarian survival factor-associated molecules that is upregulated in ovarian tumor cells. For example, the therapeutic agent can be an siRNA that interferes with mRNA expression of one of the disclosed ovarian survival factor-associated molecules that are involved in angiogenesis, such as a molecule involved in regulating cell motility, cell proliferation, cell adhesion or tube formation, thereby inhibiting cell motility, cell proliferation or tube formation. For example, the agent is an siRNA that inhibitor reduces expression of MAGP2. In additional examples, a composition includes at least two therapeutic agents such as two specific siRNAs that each bind to their respective ovarian survival factor-associated nucleotide sequences and inhibit ovarian tumor growth in a subject. For example, the composition includes MAGP2, PTPRD, KLB, TWIST1 and MMP13 siRNAs.

### Treatment of Ovarian Cancer by Altering Activity of an Ovarian Survival Factor-Associated Molecule

In several examples, decreasing the biological activity of one or more ovarian survival factor-associated molecules that are upregulated in an ovarian tumor can be used to treat a tumor. Treatment of a tumor by reducing the number of upregulated ovarian survival factor-associated molecules can include delaying the development of the tumor in a subject (such as preventing metastasis of a tumor). Treatment of a tumor also includes reducing signs or symptoms associated with the presence of such a tumor (for example by reducing the size or volume of the tumor or a metastasis thereof). Such reduced growth can in some examples decrease or slow metastasis of the tumor, or reduce the size or volume of the tumor by at least 10%, at least 20%, at least 50%, or at least 75%. For example, ovarian survival factor-associated molecules involved in angiogenesis, such as molecules involved in promoting cell proliferation, cell motility or tube formation can be inhibited to treat an ovarian tumor, such as those provided in any of Tables 1 and 2. In other examples, ovarian tumor growth is reduced or inhibited by reducing expression of ovarian survival factor-associated molecules provided in Table 1 or 2 that are upregulated in ovarian tumor cells. In further examples, reduction of ovarian survival factor-associated molecules includes reducing the invasive activity of the tumor in the subject. In some examples, treatment using the methods disclosed herein prolongs the time of survival of the subject.

### Therapeutic Agents

Therapeutic agents are agents that when administered in therapeutically effective amounts induce the desired response (e.g., treatment of a tumor). In one example, therapeutic agents are specific binding agents that bind with higher affinity to a molecule of interest, than to other molecules. For example, a specific binding agent can be one that binds with high affinity to one of the genes or gene products of the ovarian survival factor-associated molecules listed in any of Tables 1 and 2, but does not substantially bind to another gene or gene product. In some examples, a specific binding agent binds to one gene listed in Tables 1 and 2 that are upregulated in ovarian tumor cells, thereby reducing or inhibiting expression of the gene, but does not bind to the other genes (or gene product) listed in such Tables. For example, the agent can interfere with gene expression (transcription, processing, translation, post-translational modification), such as, by interfering with the gene's mRNA and blocking translation of the gene product or by post-translational modification of a gene product, or by causing changes in intracellular localization. In another example, a specific binding agent binds to a protein encoded by of one of the genes listed in Table 1 or 2 with a binding affinity in the range of 0.1 to 20 nM and reduces or inhibits the activity of such protein.

Examples of specific binding agents include siRNAs, antibodies, ligands, recombinant proteins, peptide mimetics, and soluble receptor fragments. One example of a specific binding agent is a siRNA. Methods of making siRNAs that can be used clinically are known in the art. Particular siRNAs and methods that can be used to produce and administer them are described in detail below. In a specific example, a specific binding agent includes a MAGP2 siRNA molecule has the following sequence: 5′-ACCGGTTAAACAATGCATTCAT-3′ (sense; SEQ ID NO: 1) and 5′-ATGAATGCATTGTTTAACCGGC-3′ (antisense; SEQ ID NO: 2).

Another specific example of a specific binding agent is an antibody, such as a monoclonal or polyclonal antibody. Methods of making antibodies that can be used clinically are known in the art. Particular antibodies and methods that can be used to produce them are described in detail below.

In a further example, small molecular weight inhibitors or antagonists of the receptor protein can be used to regulate activity such as the expression or production of ovarian survival factor-associated molecules. In a particular example, small molecular weight inhibitors or antagonists of the proteins encoded by the genes listed in Table 1 or 2 are employed.

Specific binding agents can be therapeutic, for example by reducing or inhibiting the biological activity of a nucleic acid or protein that is associated with ovarian tumor survival. For example, a specific binding agent that binds with high affinity to a gene listed in Tables 1 or 2 that are upregulated in ovarian tumor cells, may substantially reduce the biological function of the gene or gene product (for example, the ability of the gene or gene product to facilitate angiogenesis). In other examples, a specific binding agent that binds with high affinity to one of the proteins encoded by the genes listed in Table 1 or 2 that are upregulated in ovarian tumor cells, may substantially reduce the biological function of the protein (for example, the ability of the protein to promote angiogenesis). Such agents can be administered in therapeutically effective amounts to subjects in need thereof, such as a subject having ovarian cancer, such as papillary serous ovarian cancer.

### Pre-Screening Therapeutic Agents

In some examples, potential therapeutic agents are initially screened for treating an ovarian tumor, such as ovarian cancer, by use of the disclosed gene expression profile (as discussed in detail below). For example, the disclosed gene expression profile can be used to identify agents capable of reducing or inhibiting ovarian cancer. In an example, the disclosed gene expression profile is used to identify compositions that can be employed to reduce or inhibit angiogenesis in ovarian tumors. In additional examples, subjects can be first pre-screened for the presence of an ovarian tumor that will respond to a particular therapeutic agent prior to receiving treatment.

### Exemplary Tumors

A tumor is an abnormal growth of tissue that results from excessive cell division. A particular example of a tumor is cancer. For example, the current application provides methods for the treatment (such as the prevention or reduction of metastasis) of tumors (such as cancers) by altering the expression/production of one or more disclosed ovarian survival factor-associated molecules. In some examples, the tumor is treated in vivo, for example in a mammalian subject, such as a human subject. Exemplary tumors that can be treated using the disclosed methods include, but are not limited to ovarian cancer, including metastases of such tumors to other organs. Generally, the tumor is an ovarian cancer, such as papillary serous ovarian cancer.

### Administration

Methods of administration of the disclosed compositions are routine, and can be determined by a skilled clinician. For example, the disclosed therapies (such as those that include a binding agent specific for one of the disclosed ovarian survival factor-associated molecules listed in Table 1 or 2) can be administered via injection, intratumorally, orally, topically, transdermally, parenterally, or via inhalation or spray. In a particular example, a composition is administered intravenously to a mammalian subject, such as a human. In another example, the composition is administered into the peritoneal cavity allowing localized tumor treatment possibly reducing side effects, while increasing response.

The therapeutically effective amount of the agents administered can vary depending upon the desired effects and the subject to be treated. In one example, the method includes daily administration of at least 1 μg of a therapeutic agent to the subject (such as a human subject). For example, a human can be administered at least 1 μg or at least 1 mg of the agent daily, such as 10 μg to 100 μg daily, 100 μg to 1000 μg daily, for example 10 μg daily, 100 μg daily, or 1000 μg daily. In one example, the subject is administered at least 1 μg (such as 1-100 μg) intravenously of the therapeutic agent (such as a composition that includes a binding agent that specifically binds to one of the disclosed ovarian survival factor-associated molecules). In one example, the subject is administered at least 1 mg intramuscularly (for example in an extremity) of such composition. The dosage can be administered in divided doses (such as 2, 3, or 4 divided doses per day), or in a single dosage daily.

In particular examples, the subject is administered the therapeutic composition that includes a binding agent specific for one of the disclosed ovarian survival factor-associated molecules on a multiple daily dosing schedule, such as at least two consecutive days, 10 consecutive days, and so forth, for example for a period of weeks, months, or years. In one example, the subject is administered the therapeutic composition that a binding agent specific for one of the disclosed ovarian survival factor-associated molecules daily for a period of at least 30 days, such as at least 2 months, at least 4 months, at least 6 months, at least 12 months, at least 24 months, or at least 36 months.

The therapeutic compositions, such as those that include a binding agent specific for one of the ovarian survival factor-associated molecules, can further include one or more biologically active or inactive compounds (or both), such as anti-neoplastic agents and conventional non-toxic pharmaceutically acceptable carriers, respectively.

In a particular example, a therapeutic composition that includes a therapeutically effective amount of a therapeutic agent (such as a binding agent specific for one of the disclosed ovarian survival factor-associated molecules) further includes one or more biologically inactive compounds. Examples of such biologically inactive compounds include, but are not limited to: carriers, thickeners, diluents, buffers, preservatives, and carriers. The pharmaceutically acceptable carriers useful for these formulations are conventional (see Remington's Pharmaceutical Sciences, by E. W. Martin, Mack Publishing Co., Easton, Pa., 19th Edition (1995)). In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can include minor amounts of non-toxic auxiliary substances, such as wetting or emulsifying agents, preservatives, and pH buffering agents and the like, for example sodium acetate or sorbitan monolaurate.

### Additional Treatments

In particular examples, prior to, during, or following administration of a therapeutic amount of an agent that reduces or inhibits ovarian cancer by decreasing biological activity of one or more of the disclosed ovarian survival factor-associated molecules, the subject can receive one or more other therapies. In one example, the subject receives one or more treatments to remove or reduce the tumor prior to administration of the disclosed therapeutic agents specific for one of the disclosed ovarian survival factor-associated molecules.

Examples of such therapies include, but are not limited to, surgical treatment for removal or reduction of the tumor (such as surgical resection, cryotherapy, or chemoembolization), as well as anti-tumor pharmaceutical treatments which can include radiotherapeutic agents, anti-neoplastic chemotherapeutic agents, antibiotics, alkylating agents and antioxidants, kinase inhibitors, and other agents. Particular examples of additional therapeutic agents that can be used include microtubule binding agents, DNA intercalators or cross-linkers, DNA synthesis inhibitors, DNA and/or RNA transcription inhibitors, antibodies, enzymes, enzyme inhibitors, and gene regulators. These agents (which are administered at a therapeutically effective amount) and treatments can be used alone or in combination. Methods and therapeutic dosages of such agents are known to those skilled in the art, and can be determined by a skilled clinician.

“Microtubule binding agent” refers to an agent that interacts with tubulin to stabilize or destabilize microtubule formation thereby inhibiting cell division. Examples of microtubule binding agents that can be used in conjunction with the disclosed therapy include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

The following classes of compounds are of use in the methods disclosed herein: suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, formestane, trichostatin and derivatives and analogs thereof. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof. Kinase inhibitors include Gleevac, Iressa, and Tarceva that prevent phosphorylation and activation of growth factors.

Other therapeutic agents, for example anti-tumor agents, that may or may not fall under one or more of the classifications above, also are suitable for administration in combination with the disclosed therapies. By way of example, such agents include adriamycin, apigenin, rapamycin, zebularine, cimetidine, and derivatives and analogs thereof.

In some examples, the subject receiving the therapeutic peptide composition (such as one including a binding agent specific for one of the disclosed ovarian survival factor-associated molecules) is also administered interleukin-2 (IL-2), for example via intravenous administration. In particular examples, IL-2 (Chiron Corp., Emeryville, Calif.) is administered at a dose of at least 500,000 IU/kg as an intravenous bolus over a 15 minute period every eight hours beginning on the day after administration of the peptides and continuing for up to 5 days. Doses can be skipped depending on subject tolerance.

In some examples, the disclosed compositions can be co-administered with a fully human antibody to cytotoxic T-lymphocyte antigen-4 (anti-CTLA-4). In some example subjects receive at least 1 mg/kg anti-CTLA-4 (such as 3 mg/kg every 3 weeks or 3 mg/kg as the initial dose with subsequent doses reduced to 1 mg/kg every 3 weeks).

In one example, at least a portion of the ovarian tumor (such as a metastatic tumor) is surgically removed (for example via cryotherapy), irradiated, chemically treated (for example via chemoembolization) or combinations thereof, prior to administration of the disclosed therapies (such as administration of a binding agent specific for one of the disclosed ovarian survival factor-associated molecules). For example, a subject having a metastatic tumor can have all or part of the tumor surgically excised prior to administration of the disclosed therapies (such as one including a binding agent specific for one of the disclosed ovarian survival factor-associated molecules). In an example, one or more chemotherapeutic agents is administered following treatment with a binding agent specific for one of the disclosed ovarian survival factor-associated molecules. In another particular example, the subject has a metastatic tumor and is administered radiation therapy, chemoembolization therapy, or both concurrently with the administration of the disclosed therapies (such as one including a binding agent specific for one of the disclosed ovarian survival factor-associated molecules).

### Generation and Administration of siRNA

In one example, therapeutic agents are siRNAs that can decrease biological activity of target sequences. One of ordinary skill in the art can readily generate siRNAs, which specifically bind to one of the disclosed ovarian survival factor-associated molecules listed in Table 1 or 2. In an example, commercially available kits, such as siRNA molecule synthesizing kits from PROMEGA® (Madison, Wis.) or AMBION® (Austin, Tex.) may be used to synthesize siRNA molecules. In another example, siRNAs are obtained from commercial sources, such as from QIAGEN®Inc (Germantown, Md.), INVITROGEN® (Carlsbad, Calif.), AMBION (Austin, Tex.), DHARMACON® (Lafayette, Colo.), SIGMA-ALDRICH® (Saint Louis, Mo.) or OPENBIOSYSTEMS® (Huntsville, Ala.). In a particular example, a MAGP2 siRNA molecule has the following sequence: 5′-ACCGGTTAAACAATGCATTCAT-3′ (sense; SEQ ID NO: 1) and 5′-ATGAATGCATTGTTTAACCGGC-3′ (antisense; SEQ ID NO: 2). In other examples, an siRNA is capable of binding to a MAGP2 nucleic acid sequence with GENBANK® Accession Nos.: NM_{—}174386, AF084918, or NM_{—}003480 all of which are incorporated by reference as provided by GENBANK® on Apr. 13, 2007.

In certain examples, expression vectors are employed to express the at least one siRNA molecule. For example, an expression vector can include a nucleic acid sequence encoding at least one siRNA molecule corresponding to at least one of the disclosed ovarian survival factor-associated molecules listed in Tables 1 and/or 2. In a particular example, the vector contains a sequence(s) encoding both strands of a siRNA molecule comprising a duplex. In another example, the vector also contains sequence(s) encoding a single nucleic acid molecule that is self-complementary and thus forms a siRNA molecule. Non-limiting examples of such expression vectors are described in Paul et al., Nature Biotechnology 19:505, 2002; Miyagishi and Taira, Nature Biotechnology 19:497, 2002; Lee et al., Nature Biotechnology 19:500, 2002; and Novina et al., Nature Medicine, online publication Jun. 3, 2003.

In some examples, siRNA molecules include a delivery vehicle such as liposomes, carriers and diluents and their salts for administration to a subject. Nucleic acid molecules can be administered to cells by a variety of methods known to those of skill in the art, including, but not restricted to, encapsulation in liposomes, by iontophoresis, or by incorporation into other delivery vehicles, such as hydrogels, cyclodextrins, biodegradable nanocapsules, and bioadhesive microspheres, or by proteinaceous vectors (see, for example, O'Hare and Normand, International PCT Publication No. WO 00/53722).

Alternatively, the nucleic acid/vehicle combination can be locally delivered by direct injection or by use of an infusion pump. Direct injection of the nucleic acid molecules of the disclosure, whether subcutaneous, intramuscular, or intradermal, can take place using standard needle and syringe methodologies, or by needle-free technologies such as those described by Barry et al., International PCT Publication No. WO 99/31262. Other delivery routes, but are not limited to, oral delivery (such as in tablet or pill form), intrathecal or intraperitoneal delivery. For example, intraperitoneal delivery can take place by injecting the treatment into the peritoneal cavity of the subject in order to directly deliver the molecules to the tumor site. More detailed descriptions of nucleic acid delivery and administration are provided in Sullivan et al., PCT WO 94/02595, Draper et al., PCT WO93/23569, Beigelman et al., PCT WO99/05094 and Klimuk et al., PCT WO99/04819.

siRNA molecules can be expressed within cells from eukaryotic promoters. Those skilled in the art will recognize that any nucleic acid can be expressed in eukaryotic cells using the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595).

In some examples, siRNA molecules are expressed from transcription units (see for example, Couture et al., 1996, TIG 12:510) inserted into DNA or RNA vectors. The recombinant vectors can be DNA plasmids or viral vectors. siRNA expressing viral vectors can be constructed based on, for example, but not limited to, adeno-associated virus, retrovirus, adenovirus, lentivirus or alphavirus. In another example, pol III based constructs are used to express siRNA nucleic acid molecules (see for example, Thompson, U.S. Pat. Nos. 5,902,880 and 6,146,886).

The recombinant vectors capable of expressing the siRNA molecules can be delivered as described above, and persist in target cells. Alternatively, viral vectors can be used that provide for transient expression of nucleic acid molecules. Such vectors can be repeatedly administered as necessary. Once expressed, the siRNA molecule interacts with the target mRNA and generates an RNAi response. Delivery of siRNA molecule expressing vectors can be systemic, such as by intravenous or intramuscular administration, by administration to target cells ex-planted from a subject followed by reintroduction into the subject, or by any other means that would allow for introduction into the desired target cell.

### Generation and Administration of Antibodies

One of ordinary skill in the art can readily generate antibodies that decrease the biological activity (for example, by specifically binding) of the disclosed ovarian survival factor-associated molecules). These antibodies can be monoclonal or polyclonal. They can be chimeric or humanized. Any functional fragment or derivative of an antibody can be used including Fab, Fab′, Fab2, Fab′2, and single chain variable regions. So long as the fragment or derivative retains specificity of binding for the ovarian survival factor-associated molecule it can be used in the methods provided herein. Antibodies can be tested for specificity of binding by comparing binding to appropriate antigen to binding to irrelevant antigen or antigen mixture under a given set of conditions. If the antibody binds to appropriate antigen at least 2, at least 5, at least 7 or 10 times more than to irrelevant antigen or antigen mixture, then it is considered to be specific.

In an example, monoclonal antibodies are generated to the ovarian survival factor-associated molecules disclosed in Table 1 or 2. These monoclonal antibodies each include a variable heavy (V_{H}) and a variable light (V_{L}) chain and specifically bind to the specific ovarian survival factor-associated molecules. For example, the antibody can bind to its specific ovarian survival factor-associated molecule with an affinity constant of at least 10⁶ M⁻¹, such as at least 10⁷ M⁻¹, at least 10⁸ M⁻¹, at least 5×10⁸ M⁻¹, or at least 10⁹ M⁻¹.

The specific antibodies can include a V_{L} polypeptide having amino acid sequences of the complementarity determining regions (CDRs) that are at least about 90% identical, such as at least about 95%, at least about 98%, or at least about 99% identical to the amino acid sequences of the specific ovarian survival factor-associated molecules and a V_{H} polypeptide having amino acid sequences of the CDRs that are at least about 90% identical, such as at least about 95%, at least about 98%, or at least about 99% identical to the amino acid sequences of the specific ovarian survival factor-associated molecules.

In one example, the sequence of the specificity determining regions of each CDR is determined. Residues that are outside the SDR (non-ligand contacting sites) are substituted. For example, in any of the CDR sequences, at most one, two or three amino acids can be substituted. The production of chimeric antibodies, which include a framework region from one antibody and the CDRs from a different antibody, is well known in the art. For example, humanized antibodies can be routinely produced. The antibody or antibody fragment can be a humanized immunoglobulin having CDRs from a donor monoclonal antibody that binds one of the disclosed ovarian survival factor-associated molecules and immunoglobulin and heavy and light chain variable region frameworks from human acceptor immunoglobulin heavy and light chain frameworks. Generally, the humanized immunoglobulin specifically binds to one of the disclosed ovarian survival factor-associated molecules with an affinity constant of at least 10⁷ M⁻¹, such as at least 10⁸ M⁻¹ at least 5×10⁸ M⁻¹ or at least 10⁹ M⁻¹.

In another example, human monoclonal antibodies to the disclosed ovarian survival factor-associated molecules in Table 1 or 2 are produced. Human monoclonal antibodies can be produced by transferring donor complementarity determining regions (CDRs) from heavy and light variable chains of the donor mouse immunoglobulin into a human variable domain, and then substituting human residues in the framework regions when required to retain affinity. The use of antibody components derived from humanized monoclonal antibodies can obviate potential problems associated with the immunogenicity of the constant regions of the donor antibody. For example, when mouse monoclonal antibodies are used therapeutically, the development of human anti-mouse antibodies (HAMA) leads to clearance of the murine monoclonal antibodies and other possible adverse events. Chimeric monoclonal antibodies, with human constant regions, humanized monoclonal antibodies, retaining only murine CDRs, and “fully human” monoclonal antibodies made from phage libraries or transgenic mice have all been used to reduce or eliminate the murine content of therapeutic monoclonal antibodies.

Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993. The antibody may be of any isotype, but in some embodiments the antibody is an IgG, including but not limited to, IgG₁, IgG₂, IgG₃ and IgG₄.

In one example, the sequence of the humanized immunoglobulin heavy chain variable region framework can be at least about 65% identical to the sequence of the donor immunoglobulin heavy chain variable region framework. Thus, the sequence of the humanized immunoglobulin heavy chain variable region framework can be at least about 75%, at least about 85%, at least about 99% or at least about 95%, identical to the sequence of the donor immunoglobulin heavy chain variable region framework. Human framework regions, and mutations that can be made in a humanized antibody framework regions, are known in the art (see, for example, in U.S. Pat. No. 5,585,089).

Antibodies, such as murine monoclonal antibodies, chimeric antibodies, and humanized antibodies, include full length molecules as well as fragments thereof, such as Fab, F(ab′)₂, and Fv, which include a heavy chain and light chain variable region and are capable of binding the epitopic determinant. These antibody fragments retain some ability to selectively bind with their antigen or receptor. These fragments include: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule can be obtained by treating whole antibody with pepsin, followed by reduction, to yield an intact light chain and a portion of the heavy chain; two Fab′ fragments are obtained per antibody molecule; (3) (Fab)₂, the fragment of the antibody that can be obtained by treating whole antibody with the enzyme pepsin without subsequent reduction; F(ab)₂ is a dimer of two Fab′ fragments held together by two disulfide bonds; (4) Fv, a genetically engineered fragment containing the variable region of the light chain and the variable region of the heavy chain expressed as two chains; and (5) Single chain antibody (such as scFv), defined as a genetically engineered molecule containing the variable region of the light chain, the variable region of the heavy chain, linked by a suitable polypeptide linker as a genetically fused single chain molecule. Methods of making these fragments are known in the art (see for example, Harlow and Lane, Antibodies: A Laboratory Manual, Cold Spring Harbor Laboratory, New York, 1988. Fv antibodies are typically about 25 kDa and contain a complete antigen-binding site with three CDRs per each heavy chain and each light chain. To produce these antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments include V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

One of skill will realize that conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions) can be made in the V_{H} and the V_{L} regions to increase yield. Conservative amino acid substitution tables providing functionally similar amino acids are well known to one of ordinary skill in the art. The following six groups are examples of amino acids that are considered to be conservative substitutions for one another: Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

Antibodies can also be obtained from commercial sources. For example, antibodies are commercially available for several of the disclosed ovarian survival factor-associated molecules including MAGP2 (Rockland Inc.; Gilbertsville, Pa.), MMP13 (ABCAM®; Cambridge, Mass.), KLM (R&D Systems; Minneapolis, Minn.), TWIST1 (Abnova Corporation; Heidelberg, Germany) and PTPRD (Santa Cruz Biotechnology, Inc.; Santa Cruz, Calif.).

### Methods of Evaluating the Effectiveness of an Ovarian Tumor Treatment

Methods are disclosed herein for determining the effectiveness of an agent for the treatment of an ovarian tumor in a subject with the ovarian tumor. In one example, the method includes detecting expression of an ovarian survival factor-associated molecule in a sample from the subject following treatment with the agent. The expression of the ovarian survival factor-associated molecule following treatment can be compared to a control. For example, an in vitro assay can be employed to compare expression of one or more ovarian survival factor-associated molecules in a sample (such as ovarian tumor epithelial cells) in the presence and absence of the test agent. An alteration, such as a decrease, in the expression of the ovarian survival factor-associated molecule following treatment relative to no treatment indicates that the agent is effective for the treatment of the ovarian tumor in the subject. For example, if gene is upregulated and agent decreases expression by at least 20%, such as at least 30%, at least 40%, at least 50%, at least 60%, or at least 70%, the treatment is effective.

In a specific example, the method includes detecting and comparing the protein expression levels of the ovarian survival factor-associated molecules. In other examples, the method includes detecting and comparing the mRNA expression levels of the ovarian survival factor-associated molecules. In certain examples, the treatment is considered effective if the expression levels are altered, such as decreased, by at least 2-fold, such as by at least 3-fold, at least 4-fold, at least 6-fold or at least 10-fold relative to a control, such as protein expression level of the ovarian survival factor-associated molecules in a subject without an ovarian tumor. The alterations in the expression of one or more of the disclosed ovarian survival factor-associated molecules can be detected at the nucleic acid or protein level as described above.

In one example, the specific ovarian survival factor-associated molecule is detected in a biological sample. In a particular example, the biological sample is a tumor biopsy. In another example, the ovarian survival factor-associated molecule is detected in a serum sample. For example, the ovarian survival factor-associated molecule is detected in a serum sample if the specific molecule is known to be secreted or located on a cell surface susceptible to enzymatic cleavage.

### Identifying Ovarian Tumor Therapeutic Agents

Methods are provided herein for identifying agents to treat an ovarian tumor, such as ovarian cancer. In one example, the method includes contacting an ovarian tumor cell with one or more test agents under conditions sufficient for the one or more test agents to alter the activity of at least one ovarian survival factor-associated molecule listed in any of Tables 1 and 2. The method can also include detecting the activity of the at least one ovarian survival factor-associated molecule in the presence and absence of the one or more test agents. The activity of the at least one ovarian survival factor-associated molecule in the presence of the one or more test agents is then compared to a control, such as the activity of the at least one ovarian survival factor-associated molecule in the absence of the one or more test agents, to determine if there is differential expression of the at least one ovarian survival factor-associated molecule. In several examples, differential expression of the ovarian survival factor-associated molecule in the presence of the agent (as compared to expression in the absence of the agent) indicates that the one or more test agents is of use to treat the ovarian tumor. For example, if the monitored ovarian survival factor-associated molecule is increased in an ovarian tumor, then a test agent that decreases the expression of such molecule can be selected to treat the ovarian tumor.

In one example, determining whether there is differential expression of one or more ovarian survival factor-associated molecules is by use of an in vitro assay. For example, an in vitro assay can be employed to compare expression of one or more ovarian survival factor-associated molecules in a sample (such as ovarian tumor epithelial cells) in the presence and absence of the test agent. In a specific example, differential expression can be determined by generating a gene expression profile for the subject. For example, a gene expression profile for the subject can be generated by using an array of molecules including an ovarian survival factor-associated expression profile as described above. Ovarian survival factor-associated molecules can include nucleic acid sequences (such as DNA, cDNA, or mRNAs) and proteins. In a specific example, detecting differential expression of the ovarian survival factor-associated molecules includes detecting differential mRNA expression of the disclosed ovarian survival factor-associated molecules. For example, such differential expression is measured by real time quantitative polymerase chain reaction or microarray analysis (as previously described). In another example, detecting differential expression of the ovarian survival factor-associated molecules includes detecting differential protein expression of the disclosed ovarian survival factor-associated molecules. For example, protein differential expression is measured by Western blot analysis or a protein microarray.

### Test Agents

The one or more test agents can be any substance, including, but not limited to, a protein (such as an antibody), a nucleic acid molecule (such as a siRNA), an organic compound, an inorganic compound, a small molecule or any other molecule of interest. In a particular example, the test agent is a siRNA that reduces or inhibits the activity (such as the expression) of one of the ovarian survival factor-associated molecules, such as MAGP2, PTPRD, KLB, TWIST1 and MMP13. For example, the siRNA is directed to an ovarian survival factor-associated molecule listed in Table 1 or 2 which is involved in angiogenesis, such as a molecule that is involved in at least one of cell proliferation, cell adhesion, tube formation or cell motility.

In other examples, the test agent is an antibody. For example, the antibody is directed to specifically bind to an ovarian survival factor-associated protein encoded by one of the genes listed in any of Tables 1 or 2. In a particular example, the antibody is directed to an ovarian survival factor-associated protein encoded by one of the genes listed in Tables 1 or 2 which is involved in angiogenesis, such as a gene that is involved in at least one of cell proliferation, cell adhesion, tube formation or cell motility.

Disclosed test agents also include aptamers. In one example, an aptamer is a single stranded nucleic acid molecule (such as, DNA or RNA) that assumes a specific, sequence dependent shape and binds to a target protein (e.g., an MAGP2 protein) with high affinity and specificity. Aptamers generally comprise fewer than 100 nucleotides, fewer than 75 nucleotides, or fewer than 50 nucleotides (such as 10 to 95 nucleotides, 25 to 80 nucleotides, 30 to 75 nucleotides, or 25 to 50 nucleotides). In a specific embodiment, a disclosed diagnostic specific binding reagent is a mirror image aptamer (also called a SPIEGELMER™). Mirror image aptamers are high affinity L enantiomeric nucleic acids (for example, L ribose or L 2′-deoxyribose units) that display high resistance to enzymatic degradation compared with D oligonucleotides (such as, aptamers). The target binding properties of aptamers and mirror image aptamers are designed by an in vitro selection process starting from a random pool of oligonucleotides, as described for example, in Wlotzka et al., Proc. Natl. Acad. Sci. 99(13):8898 8902, 2002. Methods of generating aptamers are known in the art (see e.g., Fitzwater and Polisky (Methods Enzymol., 267:275-301, 1996; Murphy et al., Nucl. Acids Res. 31:e110, 2003).

In another example, an aptamer is a peptide aptamer that binds to a target protein (e.g., a MAGP2 protein) with high affinity and specificity. Peptide aptamers can include a peptide loop (e.g., which is specific for the MAGP2 protein) attached at both ends to a protein scaffold. This double structural constraint greatly increases the binding affinity of the peptide aptamer to levels comparable to an antibody's (nanomolar range). The variable loop length is typically 8 to 20 amino acids (e.g., 8 to 12 amino acids), and the scaffold may be any protein which is stable, soluble, small, and non-toxic (e.g., thioredoxin-A, stefin A triple mutant, green fluorescent protein, eglin C, and cellular transcription factor Sp1). Peptide aptamer selection can be made using different systems, such as the yeast two-hybrid system (e.g., Gal4 yeast-two-hybrid system) or the LexA interaction trap system.

### Altering Ovarian Survival Factor-Associated Molecules' Activity

In one example, an alteration in the activity of one or more of the disclosed ovarian survival factor-associated molecules includes an increase or decrease in production of a gene product, such as RNA or protein, relative to a control or reference value (or range of values). For example, an alteration can include processes that downregulate or decrease transcription of a gene or translation of mRNA. Gene downregulation includes any detectable decrease in the production of a gene product. In certain examples, production/expression of a gene product decreases by at least 2-fold, for example at least 3-fold, at least 4-fold, at least 6-fold, or at least 10-fold as compared to a control. For example, a decrease in one or more of the disclosed ovarian survival factor-associated molecules upregulated in ovarian tumor epithelial cells (such as MAGP2, PTPRD, KLB, TWIST1 and MMP13), is indicative of an agent that is effective at treating ovarian cancer.

The disclosure is further illustrated by the following non-limiting Examples.

### EXAMPLES

### Example 1

### Gene Signature Predictive for Survival in Subjects with Advanced Papillary Serous Ovarian Cancer

This example provides a gene signature predictive for survival in subjects with advanced papillary serous ovarian cancer.

Tissue Samples. Tissue specimens were obtained from sixty previously untreated ovarian cancer patients, who were hospitalized at the Brigham and Women's hospital between 1990 and 2000. All patients had stages III, grade III serous type of ovarian cancer as determined according to the International Federation of Gynecology and Obstetrics (FIGO) standards.

Microdissection and total RNA extraction. Frozen sections (7 μm) were affixed to FRAME Slides (Leica, Germany), fixed in 70% alcohol for 30 seconds, stained by 1% methylgreen, washed in water and air-dried. Microdissection was performed using a MD LMD laser microdissecting microscope (Leica, Germany). Epithelial tumor cells were selectively procured by activation of the laser. Approximately 5,000 tumor cells were dissected in each case. They were lyzed immediately in 65 μl RLT lysis buffer and RNA was extracted and purified by the RNeasy Micro Kit according to the manufacturer's protocol (Qiagen; Valencia, Calif.). Purified total RNA was quantified by the RiboGreen RNA Quantitation system (Molecular probes; Oregon, Calif.).

AFFYMETRIX® GENECHIP® hybridization and image acquisition. Total RNA quality was checked by a BioAnalyzer (Agilent, Palo Alto, Calif.) before further manipulation. Two rounds of amplification were used as previously described (Bonome et al., Cancer Res. 65: 10602-10612, 2005). Briefly, during first round first and second strand cDNA synthesis, 25 ng of total RNA was reverse transcribed using the Two-Cycle cDNA Synthesis Kit (AFFYMETRIX®, Santa Clara, Calif.) and oligo-dT24-T7 primer according to the manufacturer's instructions. First round amplification was completed using the T7 promoter coupled double stranded cDNA as template and the MEGAscript T7 Kit (Ambion, Inc., Austin, Tex.). Following clean up of the cRNA with a GENECHIP® Sample Cleanup Module IVT column (AFFYMETRIX®, Santa Clara, Calif.), second round double stranded cDNA was purified using a GENECHIP® Sample Cleanup Module cDNA column (AFFYMETRIX®, Santa Clara, Calif.) and amplified with the IVT Labeling Kit (AFFYMETRIX®, Santa Clara, Calif.). A 15.0 μg aliquot of labeled product was fragmented by heat and ion-mediated hydrolysis and hybridized to a U133 Plus 2.0 oligonucleotide array (AFFYMETRIX®, Santa Clara, Calif.) which comprises over 1,300,000 unique oligonucleotide features covering more than 47,000 transcripts and variants in a single chip. Arrays were scanned using the laser confocal GENECHIP®Scanner 3000 (AFFYMETRIX®, Santa Clara, Calif.).

Microarray survival analysis. Low-level analysis included array normalization and estimation of expression level. This was accomplished by invariant set normalization to adjust the overall signal level of the arrays to the same level for further comparison (Sorlie et al., Proc. Natl. Acad. Sci. U.S.A. 98: 10869-10874, 2001). A model-based approach was employed to calculate the gene expression level. The low-level analysis was conducted using dChip software (Li and Wong, Proc. Natl. Acad. Sci. U.S.A. 98: 31-36, 2001).

The outcome of interest was time-to-event (death) with possible censoring, hence, simple classification methods were not used in analyzing this type of data. Instead, a “semi-supervised” method was modified and employed. The detailed procedure involves two stages. In stage 1, supervised dimension reduction was applied by fitting univariate Cox model for each gene. To ensure the results were not driven by outlying samples, a jackknife procedure was utilized, and only those genes with a consistent large Cox score were included into the signature gene list. In addition, censoring and debulking status were factored in as covariates. The number of genes included in the prediction model, denoted by n_{g}, is an arbitrary parameter. Here, situations were surveyed when n_{g}=100, 200 or 300. In stage 2, the dimension was further reduced from 200 to 5 by using principal components (PC) technique. PC is an unsupervised machine learning method based on the spectral decomposition of covariance matrix of the data (expression of signature genes), X′X. The number of PC, denoted by nₚ, was set as 5. Again, nₚ=4 or 6 was surveyed to investigate how this parameter affected our results. The first 5 PCs captured about 90% of the information of the signature genes. A prediction model was then built using multivariate Cox regression, where independent variables included the first 5 PCs. Based on this model, the relative hazard and survival of future patients were predicted according to their gene expression and debulking status.

Standard leave-one-out validation was employed to evaluate the prediction model. This procedure was conducted in 53 iteration loops. In each iteration, one sample was reserved for testing, and the remaining 52 patients were used to establish the prediction model following the above described “semi-supervised” method. The reserved patients had no contribution to the prediction model, based on which the relative hazard of this patient was predicted. By these methods, the 53 predicted hazards were obtained. Then, the subjects were equally divided to low- and high-risk groups according to whether their hazard were less or greater than the sample median. Finally, a non-parametric log rank test was used to compare the survival between the two groups of subjects.

Quantitative PCR analyses. Real-time PCR was performed on amplified product from 53 specimens using primer sets specific for 11 selected genes, including MAGP2, and the house keeping genes GAPDH, GUSB, and Cyclophillin in an iCycler iQ Real-Time PCR Detection System (Bio-Rad Laboratories, Hercules, Calif.) as previously described (Wamunyokoli et al., Clin. Cancer Res. 12: 690-700, 2006). Briefly, 100 ng of amplified copy RNA (cRNA) was transcribed and PCR amplified using the QuantiTect SYBR Green RT-PCR Kit (Qiagen Inc., Valencia, Calif.). The reaction was incubated at 50° C. for 30 minutes, 95° C. for 15 minutes, and PCR cycled 45 times at 94° C. for 15 seconds, 55° C. for 30 seconds, and finally at 72° C. for 30 seconds. To calculate the relative expression for each gene, the 2^{-ΔΔ}_{T} method was used averaging the C_{T} values for the three housekeeping genes for a single reference gene value (Livak and Schmittgen, Methods 24: 402-408, 2001).

Using the methods described above, 53 stage III, grade 3 primary tumor specimens were identified from patients with papillary serous tumors of the ovary whose survival spanned a spectrum of 145 months. The average age was 61.9 years (SD=12.7), with an average survival time of 40.5 months following surgery (SD=41.3 months). Among these patients, 12 were still alive when the data was analyzed, and 11 patients were sub-optimally debulked. All specimens were analyzed as pure, micro-dissected epithelial cell populations. Total RNA isolated from these specimens was amplified and hybridized to AFFYMETRIX® U133 2.0 Plus GENECHIP® oligonucleotide microarrays.

To derive the predictor, a two-step “semi-supervised” approach was used to identify and then validate the survival signature. In the first stage of the procedure, a univariate Cox proportional hazards model was applied to dChip PM-only normalized expression data in to identify genes related to patient survival. To prevent the impact of outlying arrays, a jackknife procedure was used, while controlling for debulking status. Only those probe sets, whose Cox hazard ratio was among the top 200 (provided in Table 1) in all 53 jack-knife iterations were considered further. In the second stage, the prediction model was established. Since all 53 patients contributed to the model building process, they could not be used for independent validation. To overcome this limitation, a leave-one-out strategy was used to validate the “semi-supervised” procedure. Table 1 provides a list of 200 genes with poor survival whose increased expression is associated with poor subject outcome.

TABLE 1
Genes associated with poor subject outcome.

| | | | | | | GO Biological |
|---|---|---|---|---|---|---|
| | | | | | Gene | Process |
| Probe Set | Locuslink | Cox score | p-value | Gene Title | Symbol | Description |
| 209758_s_at | 8076 | 30.3422259 | 3.6216E−08 | Microfibril associated | MAGP2 | motility |
| | | | | glycoprotein 2 | | |
| 214043_at | 5789 | 20.8228042 | 5.038E−06 | protein tyrosine | PTPRD | protein amino acid |
| | | | | phosphatase, receptor | | dephosphorylation; |
| | | | | type, D | | phosphate |
| | | | | | | metabolism; |
| | | | | | | transmembrane |
| | | | | | | receptor protein |
| | | | | | | tyrosine |
| | | | | | | phosphatase |
| | | | | | | signaling pathway; |
| | | | | | | protein amino acid |
| | | | | | | dephosphorylation |
| 235708_at | 152831 | 19.2443655 | 1.1501E−05 | klotho beta | KLB | carbohydrate |
| | | | | | | metabolism |
| 213943_at | 7291 | 18.5773981 | 1.6314E−05 | twist homolog 1 | TWIST1 | negative regulation |
| | | | | (acrocephalosyndactyly | | of transcription |
| | | | | 3; Saethre-Chotzen | | from RNA |
| | | | | syndrome) | | polymerase II |
| | | | | (Drosophila) | | promoter; skeletal |
| | | | | | | development; |
| | | | | | | regulation of |
| | | | | | | transcription, |
| | | | | | | DNA-dependent; |
| | | | | | | chromosome |
| | | | | | | organization and |
| | | | | | | biogenesis (sensu |
| | | | | | | Eukaryota); |
| | | | | | | morphogenesis; |
| | | | | | | cell differentiation; |
| | | | | | | transcription; |
| | | | | | | development; |
| | | | | | | regulation of |
| | | | | | | transcription |
| 205959_at | 4322 | 17.5283893 | 2.8305E−05 | matrix | MMP13 | peptidoglycan |
| | | | | metallopeptidase 13 | | metabolism; |
| | | | | (collagenase 3); matrix | | proteolysis; |
| | | | | metallopeptidase 13 | | proteolysis; |
| | | | | (collagenase 3) | | collagen |
| | | | | | | catabolism |
| 203439_s_at | 8614 | 17.4529197 | 2.9451E−05 | stanniocalcin 2 | STC2 | cell surface |
| | | | | | | receptor linked |
| | | | | | | signal |
| | | | | | | transduction; cell- |
| | | | | | | cell signaling; |
| | | | | | | response to |
| | | | | | | nutrient |
| 213125_at | 25903 | 17.3553477 | 3.1002E−05 | olfactomedin-like 2B | OLFML2B | — |
| 220351_at | 51554 | 17.2748533 | 3.2344E−05 | chemokine (C-C | CCRL1 | chemotaxis; |
| | | | | motif) receptor-like 1 | | immune response; |
| | | | | | | signal |
| | | | | | | transduction; G- |
| | | | | | | protein coupled |
| | | | | | | receptor protein |
| | | | | | | signaling pathway; |
| | | | | | | G-protein coupled |
| | | | | | | receptor protein |
| | | | | | | signaling pathway |
| 229088_at | | 16.4611352 | 4.9658E−05 | — | — | — |
| 210135_s_at | 6474 | 16.4271789 | 5.0555E−05 | short stature | SHOX2 | Skeletal |
| | | | | homeobox 2 | | development; |
| | | | | | | regulation of |
| | | | | | | transcription, |
| | | | | | | DNA-dependent; |
| | | | | | | development; |
| | | | | | | nervous system |
| | | | | | | development; heart |
| | | | | | | development; |
| | | | | | | regulation of |
| | | | | | | transcription |
| 229404_at | 117581 | 16.3302987 | 5.3206E−05 | twist homolog 2 | TWIST2 | cell differentiation; |
| | | | | (Drosophila) | | negative regulation |
| | | | | | | of osteoblast |
| | | | | | | differentiation; |
| | | | | | | negative regulation |
| | | | | | | of transcription, |
| | | | | | | DNA-dependent; |
| | | | | | | regulation of |
| | | | | | | transcription; |
| | | | | | | transcription; |
| | | | | | | regulation of |
| | | | | | | transcription, |
| | | | | | | DNA-dependent; |
| | | | | | | development |
| 228376_at | | 16.2353849 | 5.5939E−05 | Glycoprotein, alpha- | GGTA1 | carbohydrate |
| | | | | galactosyltransferase 1 | | metabolism |
| 210244_at | 820 | 15.7039093 | 7.4071E−05 | cathelicidin | CAMP | response to pest, |
| | | | | antimicrobial peptide | | pathogen or |
| | | | | | | parasite; defense |
| | | | | | | response to |
| | | | | | | bacteria; defense |
| | | | | | | response; defense |
| | | | | | | response to |
| | | | | | | bacteria |
| 205066_s_at | 5167 | 15.5669611 | 7.9634E−05 | ectonucleotide | ENPP1 | generation of |
| | | | | pyrophosphatase/phosphodiesterase 1 | | precursor |
| | | | | | | metabolites and |
| | | | | | | energy; phosphate |
| | | | | | | metabolism; |
| | | | | | | response to |
| | | | | | | nutrient; |
| | | | | | | nucleotide |
| | | | | | | metabolism |
| 226084_at | 4131 | 15.2981822 | 9.1805E−05 | microtubule-associated | MAP1B | — |
| | | | | protein 1B | | |
| 244056_at | | 15.1074527 | 0.00010156 | surfactant associated | SFTPG | — |
| | | | | protein G | | |
| 211372_s_at | 7850 | 14.9309959 | 0.00011152 | interleukin 1 receptor, | IL1R2 | immune response |
| | | | | type II | | |
| 232523_at | 84466 | 14.7084083 | 0.00012549 | multiple EGF-like- | MEGF10 | lipid catabolism |
| | | | | domains 10 | | |
| 204844_at | 2028 | 14.4543316 | 0.0001436 | glutamyl | ENPEP | proteolysis; cell- |
| | | | | aminopeptidase | | cell signaling; cell |
| | | | | (aminopeptidase A) | | proliferation; cell |
| | | | | | | migration; cell |
| | | | | | | proliferation |
| 232914_s_at | 54843 | 13.9579233 | 0.00018695 | synaptotagmin-like 2 | SYTL2 | transport; |
| | | | | | | intracellular |
| | | | | | | protein transport; |
| | | | | | | vesicle-mediated |
| | | | | | | transport |
| 204130_at | 3291 | 13.9328585 | 0.00018946 | hydroxysteroid (11- | HSD11B2 | glucocorticoid |
| | | | | beta) dehydrogenase 2 | | biosynthesis; cell- |
| | | | | | | cell signaling; |
| | | | | | | metabolism |
| 1556567_at | 4676 | 13.665082 | 0.00021848 | nucleosome assembly | NAP1L4 | nucleosome |
| | | | | protein 1-like 4 | | assembly; |
| | | | | | | nucleosome |
| | | | | | | assembly |
| 219213_at | 58494 | 13.6092626 | 0.00022507 | junctional adhesion | JAM2 | cell-cell adhesion |
| | | | | molecule 2 | | |
| 229659_s_at | 5284 | 13.581612 | 0.00022841 | Transcribed locus | — | — |
| 218469_at | 26585 | 13.2877602 | 0.00026714 | gremlin 1, cysteine | GREM1 | development; |
| | | | | knot superfamily, | | nervous system |
| | | | | homolog (Xenopus | | development |
| | | | | laevis) | | |
| 227376_at | | 13.2603637 | 0.00027108 | Transcribed locus | — | — |
| 1552430_at | 116966 | 13.0126675 | 0.00030939 | WD repeat domain 17 | WDR17 | — |
| 231130_at | 51661 | 12.9067851 | 0.00032739 | FK506 binding protein 7 | FKBP7 | protein folding |
| 227642_at | 29842 | 12.815084 | 0.00034384 | Transcription factor | TFCP2L1 | transcription; |
| | | | | CP2-like 1 | | regulation of |
| | | | | | | transcription from |
| | | | | | | RNA polymerase |
| | | | | | | II promoter; |
| | | | | | | steroid |
| | | | | | | biosynthesis; |
| | | | | | | pregnancy; |
| | | | | | | regulation of |
| | | | | | | transcription, |
| | | | | | | DNA-dependent |
| 1556283_s_at | 26127 | 12.723609 | 0.00036107 | FGFR1 oncogene | FGFR1OP2 | — |
| | | | | partner 2 | | |
| 225496_s_at | 54843 | 12.4726899 | 0.00041295 | synaptotagmin-like 2 | SYTL2 | transport; |
| | | | | | | intracellular |
| | | | | | | protein transport; |
| | | | | | | vesicle-mediated |
| | | | | | | transport |
| 222196_at | | 12.4168756 | 0.00042547 | hypothetical protein | LOC286434 | — |
| | | | | LOC286434 | | |
| 223472_at | 7468 | 12.4168362 | 0.00042548 | Wolf-Hirschhorn | WHSC1 | regulation of |
| | | | | syndrome candidate 1 | | transcription, |
| | | | | | | DNA-dependent; |
| | | | | | | morphogenesis |
| 203700_s_at | 1734 | 12.1284392 | 0.00049659 | deiodinase, | DIO2 | selenocysteine |
| | | | | iodothyronine, type II | | incorporation; |
| | | | | | | thyroid hormone |
| | | | | | | generation; |
| | | | | | | hormone |
| | | | | | | biosynthesis |
| 226612_at | 134111 | 11.9566355 | 0.00054453 | similar to CG4502-PA | FLJ25076 | ubiquitin cycle |
| 232578_at | 51208 | 11.7918564 | 0.0005949 | claudin 18 | CLDN18 | calcium- |
| | | | | | | independent cell- |
| | | | | | | cell adhesion |
| 238865_at | 132430 | 11.7915148 | 0.00059501 | MRNA; cDNA | — | — |
| | | | | DKFZp686J06116 | | |
| | | | | (from clone | | |
| | | | | DKFZp686J06116) | | |
| 238717_at | | 11.7401397 | 0.00061166 | Similar to | LOC389906 | — |
| | | | | Serine/threonine- | | |
| | | | | protein kinase PRKX | | |
| | | | | (Protein kinase PKX1) | | |
| 238580_at | | 11.5652315 | 0.00067197 | Chromosome 4 open | C4orf8 | — |
| | | | | reading frame 8 | | |
| 218240_at | 28511 | 11.2978628 | 0.00077596 | NFKB inhibitor | NKIRAS2 | I-kappaB |
| | | | | interacting Ras-like 2 | | kinase/NF-kappaB |
| | | | | | | cascade; small |
| | | | | | | GTPase mediated |
| | | | | | | signal transduction |
| 206026_s_at | 7130 | 11.2568553 | 0.00079329 | tumor necrosis factor, | TNFAIP6 | inflammatory |
| | | | | alpha-induced protein 6 | | response; cell |
| | | | | | | adhesion; signal |
| | | | | | | transduction; cell- |
| | | | | | | cell signaling |
| 203735_x_at | 8496 | 11.2290164 | 0.00080528 | PTPRF interacting | PPFIBP1 | cell adhesion; |
| | | | | protein, binding | | DNA integration |
| | | | | protein 1 (liprin beta | | |
| | | | | 1) | | |
| 227235_at | | 11.2254703 | 0.00080682 | Guanylate cyclase 1, | GUCY1A3 | cGMP |
| | | | | soluble, alpha 3 | | biosynthesis; nitric |
| | | | | | | oxide mediated |
| | | | | | | signal |
| | | | | | | transduction; |
| | | | | | | circulation; |
| | | | | | | intracellular |
| | | | | | | signaling cascade; |
| | | | | | | cyclic nucleotide |
| | | | | | | biosynthesis |
| 231001_at | | 11.2243374 | 0.00080732 | similar to RIKEN | LOC387758 | — |
| | | | | cDNA 1110018M03 | | |
| 203035_s_at | 10401 | 11.0801738 | 0.00087256 | protein inhibitor of | PIAS3 | transcription; |
| | | | | activated STAT, 3 | | regulation of |
| | | | | | | transcription, |
| | | | | | | DNA-dependent; |
| | | | | | | ubiquitin cycle |
| 226777_at | 8038 | 10.9499732 | 0.00093605 | CDNA FLJ31066 fis, | — | — |
| | | | | clone | | |
| | | | | HSYRA2001153 | | |
| 230440_at | 84627 | 10.926771 | 0.00094785 | zinc finger protein 469 | ZNF469 | transcription; |
| | | | | | | regulation of |
| | | | | | | transcription, |
| | | | | | | DNA-dependent |
| 1564307_a_at | 144568 | 10.8847449 | 0.0009696 | alpha-2- | A2ML1 | — |
| | | | | macroglobulin-like 1 | | |
| 230109_at | 27115 | 10.7268669 | 0.00105591 | phosphodiesterase 7B | PDE7B | signal |
| | | | | | | transduction; |
| | | | | | | synaptic |
| | | | | | | transmission; |
| | | | | | | signal transduction |
| 228776_at | | 10.6334627 | 0.0011106 | gap junction protein, | GJA7 | transport; muscle |
| | | | | alpha 7, 45 kDa | | contraction; |
| | | | | (connexin 45) | | intercellular |
| | | | | | | junction assembly; |
| | | | | | | cell |
| | | | | | | communication |
| 231568_at | 255313 | 10.6177243 | 0.00112009 | hypothetical protein | RP6- | — |
| | | | | LOC255313; similar | 166C19.1; | |
| | | | | to hypothetical protein | LOC653266; | |
| | | | | LOC255313; similar | LOC653273; | |
| | | | | to hypothetical protein | LOC653278; | |
| | | | | LOC255313; similar | LOC653282; | |
| | | | | to hypothetical protein | LOC653285; | |
| | | | | LOC255313; similar | LOC653289; | |
| | | | | to hypothetical protein | LOC653290; | |
| | | | | LOC255313; similar | LOC653294; | |
| | | | | to hypothetical protein | LOC653295; | |
| | | | | LOC255313; similar | LOC653296 | |
| | | | | to hypothetical protein | | |
| | | | | LOC255313; similar | | |
| | | | | to hypothetical protein | | |
| | | | | LOC255313; similar | | |
| | | | | to hypothetical protein | | |
| | | | | LOC255313; similar | | |
| | | | | to hypothetical protein | | |
| | | | | LOC255313; similar | | |
| | | | | to hypothetical protein | | |
| | | | | LOC255313 | | |
| 204690_at | 9482 | 10.5305013 | 0.0011742 | syntaxin 8 | STX8 | transport; transport |
| 222834_s_at | 55970 | 10.4678274 | 0.00121471 | guanine nucleotide | GNG12 | signal |
| | | | | binding protein (G | | transduction; G- |
| | | | | protein), gamma 12 | | protein coupled |
| | | | | | | receptor protein |
| | | | | | | signaling pathway; |
| | | | | | | signal transduction |
| 204750_s_at | 1824 | 10.3669462 | 0.00128292 | desmocollin 2 | DSC2 | cell adhesion; |
| | | | | | | homophilic cell |
| | | | | | | adhesion; cell |
| | | | | | | adhesion |
| 219134_at | 64123 | 10.2617771 | 0.00135815 | EGF, latrophilin and | ELTD1 | signal |
| | | | | seven transmembrane | | transduction; |
| | | | | domain containing 1 | | neuropeptide |
| | | | | | | signaling pathway; |
| | | | | | | G-protein coupled |
| | | | | | | receptor protein |
| | | | | | | signaling pathway; |
| | | | | | | G-protein coupled |
| | | | | | | receptor protein |
| | | | | | | signaling pathway |
| 236594_at | 2314 | 10.2341795 | 0.00137862 | lethal giant larvae | LLGL1 | protein complex |
| | | | | homolog 1 | | assembly; cortical |
| | | | | (Drosophila) | | actin cytoskeleton |
| | | | | | | organization and |
| | | | | | | biogenesis |
| 205749_at | 1543 | 10.224472 | 0.00138589 | cytochrome P450, | CYP1A1 | electron transport |
| | | | | family 1, subfamily A, | | |
| | | | | polypeptide 1 | | |
| 221234_s_at | 60468 | 10.1864348 | 0.00141478 | BTB and CNC | BACH2 | transcription; |
| | | | | homology 1, basic | | regulation of |
| | | | | leucine zipper | | transcription, |
| | | | | transcription factor 2; | | DNA-dependent |
| | | | | BTB and CNC | | |
| | | | | homology 1, basic | | |
| | | | | leucine zipper | | |
| | | | | transcription factor 2 | | |
| 221942_s_at | 2982 | 10.1321318 | 0.00145707 | guanylate cyclase 1, | GUCY1A3 | cGMP |
| | | | | soluble, alpha 3 | | biosynthesis; nitric |
| | | | | | | oxide mediated |
| | | | | | | signal |
| | | | | | | transduction; |
| | | | | | | circulation; |
| | | | | | | intracellular |
| | | | | | | signaling cascade; |
| | | | | | | cyclic nucleotide |
| | | | | | | biosynthesis |
| 239744_at | | 10.1262951 | 0.00146169 | Regulator of G-protein | RGS3 | inactivation of |
| | | | | signalling 3 | | MAPK activity; |
| | | | | | | regulation of G- |
| | | | | | | protein coupled |
| | | | | | | receptor protein |
| | | | | | | signaling pathway; |
| | | | | | | negative regulation |
| | | | | | | of signal |
| | | | | | | transduction |
| 219700_at | 57125 | 10.0282527 | 0.00154157 | plexin domain | PLXDC1 | development |
| | | | | containing 1 | | |
| 203214_x_at | 983 | 9.8957425 | 0.00165662 | cell division cycle 2, | CDC2 | regulation of |
| | | | | G1 to S and G2 to M | | progression |
| | | | | | | through cell cycle; |
| | | | | | | protein amino acid |
| | | | | | | phosphorylation; |
| | | | | | | cell cycle; mitosis; |
| | | | | | | traversing start |
| | | | | | | control point of |
| | | | | | | mitotic cell cycle; |
| | | | | | | cell division |
| 206209_s_at | 762 | 9.86927525 | 0.00168062 | carbonic anhydrase IV | CA4 | one-carbon |
| | | | | | | compound |
| | | | | | | metabolism; visual |
| | | | | | | perception; |
| | | | | | | response to |
| | | | | | | stimulus |
| 238934_at | | 9.84666618 | 0.0017014 | Transcribed locus | — | — |
| 214199_at | 6441 | 9.80488575 | 0.00174049 | surfactant, pulmonary- | SFTPD | regulation of |
| | | | | associated protein D | | cytokine |
| | | | | | | production; |
| | | | | | | regulation of |
| | | | | | | cytokine |
| | | | | | | production; |
| | | | | | | oxygen and |
| | | | | | | reactive oxygen |
| | | | | | | species |
| | | | | | | metabolism; |
| | | | | | | oxygen and |
| | | | | | | reactive oxygen |
| | | | | | | species |
| | | | | | | metabolism; |
| | | | | | | phosphate |
| | | | | | | transport; receptor |
| | | | | | | mediated |
| | | | | | | endocytosis; |
| | | | | | | receptor mediated |
| | | | | | | endocytosis; |
| | | | | | | respiratory gaseous |
| | | | | | | exchange; |
| | | | | | | antimicrobial |
| | | | | | | humoral response |
| | | | | | | (sensu Vertebrata); |
| | | | | | | negative regulation |
| | | | | | | of T cell |
| | | | | | | proliferation; |
| | | | | | | negative regulation |
| | | | | | | of T cell |
| | | | | | | proliferation; |
| | | | | | | surfactant |
| | | | | | | homeostasis; |
| | | | | | | surfactant |
| | | | | | | homeostasis; |
| | | | | | | negative regulation |
| | | | | | | of interleukin-2 |
| | | | | | | biosynthesis; |
| | | | | | | negative regulation |
| | | | | | | of interleukin-2 |
| | | | | | | biosynthesis; |
| | | | | | | innate immune |
| | | | | | | response; innate |
| | | | | | | immune response; |
| | | | | | | macrophage |
| | | | | | | chemotaxis; |
| | | | | | | macrophage |
| | | | | | | chemotaxis; |
| | | | | | | alveolus |
| | | | | | | development; |
| | | | | | | alveolus |
| | | | | | | development; |
| | | | | | | positive regulation |
| | | | | | | of phagocytosis; |
| | | | | | | positive regulation |
| | | | | | | of phagocytosis; |
| | | | | | | regulation of liquid |
| | | | | | | surface tension |
| 207712_at | 574 | 9.79068135 | 0.00175398 | B melanoma antigen | BAGE | — |
| 213001_at | 23452 | 9.7894667 | 0.00175514 | angiopoietin-like 2 | ANGPTL2 | development |
| 234635_at | | 9.64110036 | 0.00190271 | keratin associated | KRTAP4-10 | — |
| | | | | protein 4-10 | | |
| 218923_at | 64173 | 9.63026343 | 0.00191397 | chitobiase, di-N- | CTBS | carbohydrate |
| | | | | acetyl- | | metabolism; chitin |
| | | | | | | catabolism; signal |
| | | | | | | transduction; G- |
| | | | | | | protein coupled |
| | | | | | | receptor protein |
| | | | | | | signaling pathway; |
| | | | | | | signal transduction |
| 212746_s_at | 9859 | 9.60912069 | 0.00193613 | centrosomal protein | CEP170 | — |
| | | | | 170 kDa | | |
| 210713_at | 6453 | 9.59888076 | 0.00194696 | intersectin 1 (SH3 | ITSN1 | regulation of Rho |
| | | | | domain protein) | | protein signal |
| | | | | | | transduction; |
| | | | | | | synaptic vesicle |
| | | | | | | endocytosis; |
| | | | | | | endocytosis |
| 242631_x_at | | 9.55008717 | 0.0019994 | deleted in liver cancer 1 | DLC1 | cytoskeleton |
| | | | | | | organization and |
| | | | | | | biogenesis; signal |
| | | | | | | transduction; |
| | | | | | | regulation of cell |
| | | | | | | adhesion; negative |
| | | | | | | regulation of cell |
| | | | | | | growth; signal |
| | | | | | | transduction |
| 1563643_at | | 9.37062889 | 0.0022049 | MRNA; cDNA | — | — |
| | | | | DKFZp686C1437 | | |
| | | | | (from clone | | |
| | | | | DKFZp686C1437) | | |
| 218820_at | 56967 | 9.34336806 | 0.00223794 | chromosome 14 open | C14orf132 | — |
| | | | | reading frame 132 | | |
| 207124_s_at | 10681 | 9.28278275 | 0.00231318 | guanine nucleotide | GNB5 | signal |
| | | | | binding protein (G | | transduction; |
| | | | | protein), beta 5 | | signal transduction |
| 212364_at | 4430 | 9.28038256 | 0.00231621 | myosin IB | MYO1B | — |
| 1568827_at | | 9.26017281 | 0.00234191 | hypothetical gene | LOC401442 | — |
| | | | | supported by | | |
| | | | | BC028401 | | |
| 211401_s_at | 2263 | 9.13756678 | 0.00250414 | fibroblast growth | FGFR2 | protein amino acid |
| | | | | factor receptor 2 | | phosphorylation; |
| | | | | (bacteria-expressed | | protein amino acid |
| | | | | kinase, keratinocyte | | phosphorylation; |
| | | | | growth factor receptor, | | cell growth |
| | | | | craniofacial dysostosis | | |
| | | | | 1, Crouzon syndrome, | | |
| | | | | Pfeiffer syndrome, | | |
| | | | | Jackson-Weiss | | |
| | | | | syndrome) | | |
| 206987_x_at | 8817 | 9.08718654 | 0.00257407 | fibroblast growth | FGF18 | signal |
| | | | | factor 18 | | transduction; cell- |
| | | | | | | cell signaling; |
| | | | | | | positive regulation |
| | | | | | | of cell |
| | | | | | | proliferation; |
| | | | | | | morphogenesis |
| 216086_at | 22987 | 9.05399584 | 0.00262121 | synaptic vesicle | SV2C | neurotransmitter |
| | | | | glycoprotein 2C | | transport; transport |
| 207481_at | 746 | 8.96044948 | 0.00275887 | — | — | — |
| 210319_x_at | 4488 | 8.9445455 | 0.00278299 | msh homeobox | MSX2 | skeletal |
| | | | | homolog 2 | | development; |
| | | | | (Drosophila) | | regulation of |
| | | | | | | transcription, |
| | | | | | | DNA-dependent; |
| | | | | | | development; |
| | | | | | | regulation of |
| | | | | | | transcription |
| 220205_at | 7179 | 8.9320957 | 0.00280203 | transmembrane | TPTE | protein amino acid |
| | | | | phosphatase with | | dephosphorylation; |
| | | | | tensin homology | | signal |
| | | | | | | transduction; |
| | | | | | | protein amino acid |
| | | | | | | dephosphorylation |
| 241676_x_at | | 8.92007557 | 0.00282053 | — | — | — |
| 237923_at | | 8.90947568 | 0.00283695 | Single-minded | SIM2 | regulation of |
| | | | | homolog 2 | | transcription, |
| | | | | (Drosophila) | | DNA-dependent; |
| | | | | | | signal |
| | | | | | | transduction; |
| | | | | | | nervous system |
| | | | | | | development; cell |
| | | | | | | differentiation; |
| | | | | | | transcription; |
| | | | | | | development; |
| | | | | | | nervous system |
| | | | | | | development; |
| | | | | | | regulation of |
| | | | | | | transcription |
| 210559_s_at | 983 | 8.90408618 | 0.00284533 | cell division cycle 2, | CDC2 | regulation of |
| | | | | G1 to S and G2 to M | | progression |
| | | | | | | through cell cycle; |
| | | | | | | protein amino acid |
| | | | | | | phosphorylation; |
| | | | | | | cell cycle; mitosis; |
| | | | | | | traversing start |
| | | | | | | control point of |
| | | | | | | mitotic cell cycle; |
| | | | | | | cell division |
| 241121_at | | 8.88554019 | 0.00287438 | — | — | — |
| 214299_at | 7156 | 8.86892128 | 0.00290067 | topoisomerase (DNA) | TOP3A | DNA topological |
| | | | | III alpha | | change; DNA |
| | | | | | | unwinding during |
| | | | | | | replication; DNA |
| | | | | | | modification; |
| | | | | | | chromosome |
| | | | | | | organization and |
| | | | | | | biogenesis (sensu |
| | | | | | | Eukaryota); |
| | | | | | | meiosis |
| 214598_at | 9073 | 8.86461196 | 0.00290752 | claudin 8 | CLDN8 | calcium- |
| | | | | | | independent cell- |
| | | | | | | cell adhesion |
| 202150_s_at | 4739 | 8.84765593 | 0.00293466 | neural precursor cell | NEDD9 | regulation of |
| | | | | expressed, | | progression |
| | | | | developmentally | | through cell cycle; |
| | | | | down-regulated 9 | | regulation of cell |
| | | | | | | growth; |
| | | | | | | cytoskeleton |
| | | | | | | organization and |
| | | | | | | biogenesis; cell |
| | | | | | | cycle; mitosis; cell |
| | | | | | | adhesion; signal |
| | | | | | | transduction; |
| | | | | | | integrin-mediated |
| | | | | | | signaling pathway; |
| | | | | | | actin filament |
| | | | | | | bundle formation; |
| | | | | | | cell division; cell |
| | | | | | | adhesion |
| 235182_at | 140862 | 8.76889953 | 0.0030641 | chromosome 20 open | C20orf82 | — |
| | | | | reading frame 82 | | |
| 230930_at | | 8.75591664 | 0.00308599 | hypothetical protein | LOC338620 | — |
| | | | | LOC338620 | | |
| 238727_at | | 8.73018612 | 0.00312985 | Hypothetical gene | LOC440934 | — |
| | | | | supported by | | |
| | | | | BC008048 | | |
| 212876_at | 8702 | 8.72639673 | 0.00313636 | UDP-Gal:betaGlcNAc | B4GALT4 | carbohydrate |
| | | | | beta 1,4- | | metabolism; |
| | | | | galactosyltransferase, | | membrane lipid |
| | | | | polypeptide 4 | | metabolism |
| 218193_s_at | 51026 | 8.71913908 | 0.00314887 | golgi transport 1 | GOLT1B | protein transport; |
| | | | | homolog B (S. cerevisiae) | | vesicle-mediated |
| | | | | | | transport; positive |
| | | | | | | regulation of I- |
| | | | | | | kappaB kinase/NF- |
| | | | | | | kappaB cascade; |
| | | | | | | transport |
| 226623_at | 220965 | 8.70236369 | 0.00317798 | phytanoyl-CoA 2- | PHYHIPL | — |
| | | | | hydroxylase | | |
| | | | | interacting protein-like | | |
| 214383_x_at | 116138 | 8.69832794 | 0.00318502 | kelch domain | KLHDC3 | meiotic |
| | | | | containing 3 | | recombination; |
| | | | | | | meiosis; central |
| | | | | | | nervous system |
| | | | | | | development; |
| | | | | | | meiotic |
| | | | | | | recombination |
| 226069_at | 144165 | 8.67608288 | 0.00322413 | prickle-like 1 | PRICKLE1 | — |
| | | | | (Drosophila) | | |
| 1558683_a_at | 8091 | 8.66973303 | 0.00323539 | high mobility group | HMGA2 | establishment |
| | | | | AT-hook 2 | | and/or |
| | | | | | | maintenance of |
| | | | | | | chromatin |
| | | | | | | architecture; |
| | | | | | | transcription; |
| | | | | | | regulation of |
| | | | | | | transcription, |
| | | | | | | DNA-dependent; |
| | | | | | | regulation of |
| | | | | | | transcription, |
| | | | | | | DNA-dependent; |
| | | | | | | chromosome |
| | | | | | | organization and |
| | | | | | | biogenesis (sensu |
| | | | | | | Eukaryota); |
| | | | | | | development |
| 234786_at | | 8.63181469 | 0.00330342 | MRNA; cDNA | — | — |
| | | | | DKFZp566E213 (from | | |
| | | | | clone | | |
| | | | | DKFZp566E213) | | |
| 211694_at | 83942 | 8.63060218 | 0.00330562 | testis-specific serine | TSSK1 | spermatogenesis; |
| | | | | kinase 1; testis-specific | | protein amino acid |
| | | | | serine kinase 1 | | phosphorylation; |
| | | | | | | protein amino acid |
| | | | | | | phosphorylation; |
| | | | | | | development; |
| | | | | | | spermatogenesis; |
| | | | | | | cell differentiation |
| 210337_s_at | 47 | 8.62875278 | 0.00330898 | ATP citrate lyase | ACLY | citrate metabolism; |
| | | | | | | ATP catabolism; |
| | | | | | | metabolism; lipid |
| | | | | | | biosynthesis; |
| | | | | | | coenzyme A |
| | | | | | | metabolism |
| 214577_at | 4131 | 8.62015481 | 0.00332464 | microtubule-associated | MAP1B | — |
| | | | | protein 1B | | |
| 228679_at | | 8.51921672 | 0.00351416 | CDNA FLJ30856 fis, | — | — |
| | | | | clone FEBRA2003258 | | |
| 209363_s_at | 9412 | 8.49203943 | 0.00356704 | SRB7 suppressor of | SURB7 | transcription; |
| | | | | RNA polymerase B | | positive regulation |
| | | | | homolog (yeast) | | of transcription |
| | | | | | | from RNA |
| | | | | | | polymerase II |
| | | | | | | promoter; |
| | | | | | | regulation of |
| | | | | | | transcription, |
| | | | | | | DNA-dependent; |
| | | | | | | regulation of |
| | | | | | | transcription from |
| | | | | | | RNA polymerase |
| | | | | | | II promoter |
| 203736_s_at | 8496 | 8.48611178 | 0.00357868 | PTPRF interacting | PPFIBP1 | cell adhesion; |
| | | | | protein, binding | | DNA integration |
| | | | | protein 1 (liprin beta | | |
| | | | | 1) | | |
| 224674_at | 80727 | 8.45613031 | 0.00363815 | tweety homolog 3 | TTYH3 | — |
| | | | | (Drosophila) | | |
| 235343_at | 79805 | 8.45405775 | 0.0036423 | — | — | — |
| 1561454_at | | 8.41967659 | 0.00371182 | CDNA clone | — | — |
| | | | | IMAGE: 5295408 | | |
| 229694_at | 55717 | 8.37282726 | 0.00380873 | bromodomain and WD | BRWD2 | — |
| | | | | repeat domain | | |
| | | | | containing 2 | | |
| 231500_s_at | 339088 | 8.37245412 | 0.00380951 | SLC7A5 pseudogene | LAT1-3TM | — |
| 213362_at | 5789 | 8.34214637 | 0.00387358 | protein tyrosine | PTPRD | protein amino acid |
| | | | | phosphatase, receptor | | dephosphorylation; |
| | | | | type, D | | phosphate |
| | | | | | | metabolism; |
| | | | | | | transmembrane |
| | | | | | | receptor protein |
| | | | | | | tyrosine |
| | | | | | | phosphatase |
| | | | | | | signaling pathway; |
| | | | | | | protein amino acid |
| | | | | | | dephosphorylation |
| 203639_s_at | 2263 | 8.33823412 | 0.00388193 | fibroblast growth | FGFR2 | protein amino acid |
| | | | | factor receptor 2 | | phosphorylation; |
| | | | | (bacteria-expressed | | protein amino acid |
| | | | | kinase, keratinocyte | | phosphorylation; |
| | | | | growth factor receptor, | | cell growth |
| | | | | craniofacial dysostosis | | |
| | | | | 1, Crouzon syndrome, | | |
| | | | | Pfeiffer syndrome, | | |
| | | | | Jackson-Weiss | | |
| | | | | syndrome) | | |
| 229569_at | | 8.32625058 | 0.00390762 | CDNA clone | — | — |
| | | | | IMAGE: 5263455 | | |
| 204612_at | 5569 | 8.32504871 | 0.00391021 | protein kinase (cAMP- | PKIA | negative regulation |
| | | | | dependent, catalytic) | | of protein kinase |
| | | | | inhibitor alpha | | activity |
| 204518_s_at | 5480 | 8.24968607 | 0.00407591 | peptidylprolyl | PPIC | protein folding; |
| | | | | isomerase C | | signal transduction |
| | | | | (cyclophilin C) | | |
| 213062_at | 123803 | 8.23017305 | 0.00411996 | N-terminal asparagine | NTAN1 | — |
| | | | | amidase | | |
| 231181_at | | 8.21722001 | 0.00414947 | Transcribed locus | — | — |
| 205331_s_at | 51308 | 8.1353313 | 0.00434109 | receptor accessory | REEP2 | — |
| | | | | protein 2 | | |
| 1553608_a_at | | 8.08604136 | 0.00446075 | chromosome 21 open | C21orf109; | — |
| | | | | reading frame 109; | LOC643720 | |
| | | | | similar to Protein | | |
| | | | | C21orf109 | | |
| 228506_at | 54780 | 8.07376291 | 0.00449108 | chromosome 10 open | C10orf86 | — |
| | | | | reading frame 86 | | |
| 240913_at | 2263 | 8.06289343 | 0.0045181 | fibroblast growth | FGFR2 | protein amino acid |
| | | | | factor receptor 2 | | phosphorylation; |
| | | | | (bacteria-expressed | | protein amino acid |
| | | | | kinase, keratinocyte | | phosphorylation; |
| | | | | growth factor receptor, | | cell growth |
| | | | | craniofacial dysostosis | | |
| | | | | 1, Crouzon syndrome, | | |
| | | | | Pfeiffer syndrome, | | |
| | | | | Jackson-Weiss | | |
| | | | | syndrome) | | |
| 203501_at | 10404 | 8.01910416 | 0.00462865 | plasma glutamate | PGCP | proteolysis; |
| | | | | carboxypeptidase | | proteolysis; |
| | | | | | | peptide |
| | | | | | | metabolism |
| 228433_at | 221442 | 8.0033655 | 0.00466905 | hypothetical protein | FLJ11236 | — |
| | | | | FLJ11236 | | |
| 229168_at | 91522 | 7.97343419 | 0.00474688 | collagen, type XXIII, | COL23A1 | phosphate |
| | | | | alpha 1 | | transport |
| 1552946_at | 163071 | 7.96136601 | 0.00477863 | zinc finger protein 114 | ZNF114 | regulation of |
| | | | | | | transcription, |
| | | | | | | DNA-dependent |
| 1560859_at | | 7.94358807 | 0.00482581 | fibroblast growth | FGFR2 | protein amino acid |
| | | | | factor receptor 2 | | phosphorylation; |
| | | | | (bacteria-expressed | | protein amino acid |
| | | | | kinase, keratinocyte | | phosphorylation; |
| | | | | growth factor receptor, | | cell growth |
| | | | | craniofacial dysostosis | | |
| | | | | 1, Crouzon syndrome, | | |
| | | | | Pfeiffer syndrome, | | |
| | | | | Jackson-Weiss | | |
| | | | | syndrome) | | |
| 228636_at | 27319 | 7.91738973 | 0.00489619 | basic helix-loop-helix | BHLHB5 | — |
| | | | | domain containing, | | |
| | | | | class B, 5 | | |
| 234584_s_at | 11101 | 7.91241034 | 0.00490968 | arginyltransferase 1 | ATE1 | ubiquitin cycle; |
| | | | | | | protein |
| | | | | | | arginylation; |
| | | | | | | regulation of |
| | | | | | | protein catabolism; |
| | | | | | | protein |
| | | | | | | arginylation |
| 230424_at | 9315 | 7.8792145 | 0.00500062 | chromosome 5 open | C5orf13 | — |
| | | | | reading frame 13 | | |
| 214595_at | 3755 | 7.82418173 | 0.00515518 | potassium voltage- | KCNG1 | ion transport; |
| | | | | gated channel, | | potassium ion |
| | | | | subfamily G, member 1 | | transport; |
| | | | | | | potassium ion |
| | | | | | | transport; transport |
| 228080_at | 143903 | 7.79173229 | 0.00524859 | layilin | LAYN | — |
| 205403_at | 7850 | 7.76748356 | 0.00531951 | interleukin 1 receptor, | IL1R2 | immune response |
| | | | | type II | | |
| 221487_s_at | 2029 | 7.76325163 | 0.00533199 | endosulfine alpha | ENSA | transport; response |
| | | | | | | to nutrient |
| 238252_at | | 7.7380028 | 0.00540706 | Arginyltransferase 1 | ATE1 | ubiquitin cycle; |
| | | | | | | protein |
| | | | | | | arginylation; |
| | | | | | | regulation of |
| | | | | | | protein catabolism; |
| | | | | | | protein |
| | | | | | | arginylation |
| 204988_at | 2244 | 7.71898629 | 0.00546431 | fibrinogen beta chain | FGB | blood coagulation; |
| | | | | | | blood pressure |
| | | | | | | regulation; positive |
| | | | | | | regulation of cell |
| | | | | | | proliferation; |
| | | | | | | blood coagulation |
| 204161_s_at | 22875 | 7.71076294 | 0.00548926 | ectonucleotide | ENPP4 | nucleotide |
| | | | | pyrophosphatase/phosphodiesterase 4 | | metabolism |
| | | | | (putative function) | | |
| 222853_at | 23767 | 7.70382397 | 0.0055104 | fibronectin leucine | FLRT3 | cell adhesion |
| | | | | rich transmembrane | | |
| | | | | protein 3 | | |
| 240964_at | | 7.67959222 | 0.00558488 | Phosphatase and | PTEN | regulation of |
| | | | | tensin homolog | | cyclin-dependent |
| | | | | (mutated in multiple | | protein kinase |
| | | | | advanced cancers 1) | | activity; protein |
| | | | | | | amino acid |
| | | | | | | dephosphorylation; |
| | | | | | | protein amino acid |
| | | | | | | dephosphorylation; |
| | | | | | | lipid metabolism; |
| | | | | | | induction of |
| | | | | | | apoptosis; cell |
| | | | | | | cycle; central |
| | | | | | | nervous system |
| | | | | | | development; heart |
| | | | | | | development; cell |
| | | | | | | proliferation; |
| | | | | | | negative regulation |
| | | | | | | of cell |
| | | | | | | proliferation; cell |
| | | | | | | migration; negative |
| | | | | | | regulation of cell |
| | | | | | | migration; |
| | | | | | | regulation of |
| | | | | | | protein stability; |
| | | | | | | negative regulation |
| | | | | | | of progression |
| | | | | | | through cell cycle; |
| | | | | | | inositol phosphate |
| | | | | | | dephosphorylation; |
| | | | | | | phosphoinositide |
| | | | | | | dephosphorylation; |
| | | | | | | negative regulation |
| | | | | | | of focal adhesion |
| | | | | | | formation; |
| | | | | | | negative regulation |
| | | | | | | of protein kinase B |
| | | | | | | signaling cascade; |
| | | | | | | protein amino acid |
| | | | | | | dephosphorylation; |
| | | | | | | regulation of |
| | | | | | | progression |
| | | | | | | through cell cycle |
| 212557_at | 26036 | 7.58246592 | 0.00589388 | zinc finger protein 451 | ZNF451 | transcription; |
| | | | | | | regulation of |
| | | | | | | transcription, |
| | | | | | | DNA-dependent; |
| | | | | | | phosphoenolpyruvate- |
| | | | | | | dependent |
| | | | | | | sugar |
| | | | | | | phosphotransferase |
| | | | | | | system |
| 244873_s_at | | 7.57190515 | 0.00592851 | Secretion regulating | SERGEF | signal |
| | | | | guanine nucleotide | | transduction; |
| | | | | exchange factor | | negative regulation |
| | | | | | | of protein secretion |
| 1554712_a_at | 219970 | 7.49552098 | 0.00618526 | glycine-N- | GLYATL2 | — |
| | | | | acyltransferase-like 2 | | |
| 244384_at | | 7.49391828 | 0.00619077 | Transcription factor | TFCP2L1 | transcription; |
| | | | | CP2-like 1 | | regulation of |
| | | | | | | transcription from |
| | | | | | | RNA polymerase |
| | | | | | | II promoter; |
| | | | | | | steroid |
| | | | | | | biosynthesis; |
| | | | | | | pregnancy; |
| | | | | | | regulation of |
| | | | | | | transcription, |
| | | | | | | DNA-dependent |
| 229947_at | | 7.43191085 | 0.00640773 | peptidase inhibitor 15 | PI15 | — |
| 234986_at | 2730 | 7.38279818 | 0.00658506 | Full-length cDNA | — | — |
| | | | | clone | | |
| | | | | CS0CAP007YJ17 of | | |
| | | | | Thymus of Homo | | |
| | | | | sapiens (human) | | |
| 201916_s_at | 11231 | 7.32600359 | 0.0067964 | SEC63-like (S. cerevisiae) | SEC63 | protein folding; |
| | | | | | | protein targeting to |
| | | | | | | membrane; protein |
| | | | | | | transport; transport |
| 205091_x_at | 5965 | 7.28450424 | 0.00695519 | RecQ protein-like | RECQL | DNA repair; DNA |
| | | | | (DNA helicase Q1- | | recombination |
| | | | | like) | | |
| 239113_at | | 7.15772162 | 0.0074642 | — | — | — |
| 207889_at | 1757 | 7.11814574 | 0.00763076 | sarcosine | SARDH | electron transport; |
| | | | | dehydrogenase | | glycine catabolism |
| 1564031_a_at | 285613 | 7.06476362 | 0.00786147 | chromosome 5 open | C5orf16 | — |
| | | | | reading frame 16 | | |
| 210568_s_at | 5965 | 7.05826427 | 0.00789004 | RecQ protein-like | RECQL | DNA repair; DNA |
| | | | | (DNA helicase Q1- | | recombination |
| | | | | like) | | |
| 227741_at | 201562 | 7.03680121 | 0.00798515 | protein tyrosine | PTPLB | — |
| | | | | phosphatase-like | | |
| | | | | (proline instead of | | |
| | | | | catalytic arginine), | | |
| | | | | member b | | |
| 1561608_at | | 7.02280571 | 0.0080478 | CDNA clone | — | — |
| | | | | IMAGE: 4826598 | | |
| 237077_at | | 7.01201954 | 0.00809643 | Acid phosphatase, | ACPP | regulation of |
| | | | | prostate | | progression |
| | | | | | | through cell cycle |
| 212365_at | 4430 | 6.99207355 | 0.00818715 | myosin IB | MYO1B | — |
| 241291_at | | 6.93789613 | 0.00843883 | CDNA FLJ36657 fis, | — | — |
| | | | | clone UTERU2001876 | | |
| 219735_s_at | 29842 | 6.87795674 | 0.00872653 | transcription factor | TFCP2L1 | transcription; |
| | | | | CP2-like 1 | | regulation of |
| | | | | | | transcription from |
| | | | | | | RNA polymerase |
| | | | | | | II promoter; |
| | | | | | | steroid |
| | | | | | | biosynthesis; |
| | | | | | | pregnancy; |
| | | | | | | regulation of |
| | | | | | | transcription, |
| | | | | | | DNA-dependent |
| 1568682_a_at | | 6.82859185 | 0.008971 | CDNA clone | — | — |
| | | | | IMAGE: 4837965 | | |
| 1562456_at | | 6.81760714 | 0.00902634 | MRNA; cDNA | — | — |
| | | | | DKFZp566C0924 | | |
| | | | | (from clone | | |
| | | | | DKFZp566C0924) | | |
| 203294_s_at | 3998 | 6.7743157 | 0.00924789 | lectin, mannose- | LMAN1 | protein folding; |
| | | | | binding, 1 | | ER to Golgi |
| | | | | | | vesicle-mediated |
| | | | | | | transport; blood |
| | | | | | | coagulation; |
| | | | | | | protein transport; |
| | | | | | | transport; ER to |
| | | | | | | Golgi vesicle- |
| | | | | | | mediated transport |
| 230166_at | 158405 | 6.72162159 | 0.0095251 | KIAA1958 | KIAA1958 | — |
| 222527_s_at | 55696 | 6.67118247 | 0.00979842 | RNA binding motif | RBM22 | — |
| | | | | protein 22 | | |
| 222344_at | 9315 | 6.6450524 | 0.00994316 | Chromosome 5 open | C5orf13 | — |
| | | | | reading frame 13 | | |
| 211376_s_at | 54780 | 6.63877718 | 0.00997824 | chromosome 10 open | C10orf86 | — |
| | | | | reading frame 86 | | |
| 1566893_at | | 6.62543288 | 0.01005327 | MRNA; cDNA | — | — |
| | | | | DKFZp667B1113 | | |
| | | | | (from clone | | |
| | | | | DKFZp667B1113) | | |
| 217672_x_at | | 6.59551672 | 0.01022359 | — | — | — |
| 242763_at | | 6.58356106 | 0.01029248 | Polycystic kidney and | PKHD1L1 | — |
| | | | | hepatic disease 1 | | |
| | | | | (autosomal recessive)- | | |
| | | | | like 1 | | |
| 236468_at | 114795 | 6.58126448 | 0.01030577 | CDNA FLJ37467 fis, | — | — |
| | | | | clone | | |
| | | | | BRAWH2011920 | | |
| 219649_at | 29929 | 6.54638788 | 0.01050973 | asparagine-linked | ALG6 | protein amino acid |
| | | | | glycosylation 6 | | N-linked |
| | | | | homolog (S. cerevisiae, | | glycosylation; |
| | | | | alpha-1,3- | | protein amino acid |
| | | | | glucosyltransferase) | | N-linked |
| | | | | | | glycosylation |
| 224608_s_at | 84313 | 6.5073704 | 0.01074283 | vacuolar protein | VPS25 | transcription; |
| | | | | sorting 25 homolog (S. cerevisiae) | | regulation of |
| | | | | | | transcription, |
| | | | | | | DNA-dependent; |
| | | | | | | protein transport; |
| | | | | | | transport |
| 205572_at | 285 | 6.4997523 | 0.01078895 | angiopoietin 2 | ANGPT2 | angiogenesis; |
| | | | | | | signal |
| | | | | | | transduction; cell |
| | | | | | | differentiation; |
| | | | | | | development |
| 221627_at | 10107 | 6.4884201 | 0.01085794 | tripartite motif- | TRIM10 | hemopoiesis |
| | | | | containing 10 | | |
| 205489_at | 1428 | 6.35495993 | 0.01170536 | crystallin, mu | CRYM | visual perception |
| 207518_at | 8526 | 6.34915095 | 0.01174375 | diacylglycerol kinase, | DGKE | protein kinase C |
| | | | | epsilon 64 kDa | | activation; |
| | | | | | | intracellular |
| | | | | | | signaling cascade; |
| | | | | | | phospholipid |
| | | | | | | biosynthesis |
| 201969_at | 4678 | 6.32156227 | 0.01192785 | nuclear autoantigenic | NASP | DNA packaging; |
| | | | | sperm protein | | spermatogenesis |
| | | | | (histone-binding) | | |
| 201127_s_at | 47 | 6.03976023 | 0.01398719 | ATP citrate lyase | ACLY | citrate metabolism; |
| | | | | | | ATP catabolism; |
| | | | | | | metabolism; lipid |
| | | | | | | biosynthesis; |
| | | | | | | coenzyme A |
| | | | | | | metabolism |
| 235875_at | | 5.95277583 | 0.01469413 | Solute carrier family 1 | SLC1A4 | transport; |
| | | | | (glutamate/neutral | | dicarboxylic acid |
| | | | | amino acid | | transport; neutral |
| | | | | transporter), member 4 | | amino acid |
| | | | | | | transport |
| 210426_x_at | 6095 | 5.92823142 | 0.01490019 | RAR-related orphan | RORA | transcription; |
| | | | | receptor A | | regulation of |
| | | | | | | transcription, |
| | | | | | | DNA-dependent; |
| | | | | | | signal transduction |
| 237548_at | | 5.83554533 | 0.01570554 | Sterile alpha motif and | ZAK | cell cycle |
| | | | | leucine zipper | | checkpoint; DNA |
| | | | | containing kinase | | damage |
| | | | | AZK | | checkpoint; |
| | | | | | | activation of |
| | | | | | | MAPKK activity; |
| | | | | | | protein amino acid |
| | | | | | | phosphorylation; |
| | | | | | | response to stress; |
| | | | | | | cell cycle; cell |
| | | | | | | cycle arrest; |
| | | | | | | protein kinase |
| | | | | | | cascade; activation |
| | | | | | | of JNK activity; |
| | | | | | | cell death; cell |
| | | | | | | proliferation; |
| | | | | | | response to |
| | | | | | | radiation; cell |
| | | | | | | differentiation; |
| | | | | | | positive regulation |
| | | | | | | of apoptosis; |
| | | | | | | protein amino acid |
| | | | | | | phosphorylation |
| 204580_at | 4321 | 5.81019339 | 0.01593354 | matrix | MMP12 | peptidoglycan |
| | | | | metallopeptidase 12 | | metabolism; |
| | | | | (macrophage elastase) | | proteolysis; cell |
| | | | | | | motility; |
| | | | | | | proteolysis |
| 1564985_a_at | 6546 | 5.75199566 | 0.01646994 | solute carrier family 8 | SLC8A1 | ion transport; |
| | | | | (sodium/calcium | | sodium ion |
| | | | | exchanger), member 1 | | transport; calcium |
| | | | | | | ion transport; |
| | | | | | | calcium ion |
| | | | | | | transport; muscle |
| | | | | | | contraction; cell |
| | | | | | | communication; |
| | | | | | | transport; sodium |
| | | | | | | ion transport |
| 231893_at | 85449 | 5.58757117 | 0.01808836 | KIAA1755 protein | RP5- | — |
| | | | | | 1054A22.3 | |
| 239447_at | | 5.37806776 | 0.02039145 | Transcribed locus | — | — |
| 1558337_at | | 5.2999604 | 0.02132591 | chromosome 12 open | C12orf9 | cell adhesion |
| | | | | reading frame 9 | | |
| 205700_at | 8630 | 5.2593173 | 0.02182958 | hydroxysteroid (17- | HSD17B6 | androgen |
| | | | | beta) dehydrogenase 6 | | biosynthesis; |
| | | | | | | androgen |
| | | | | | | catabolism; |
| | | | | | | metabolism |
| 211754_s_at | 10478 | 5.22636368 | 0.022247 | solute carrier family | SLC25A17 | transport; |
| | | | | 25 (mitochondrial | | mitochondrial |
| | | | | carrier; peroxisomal | | transport; transport |
| | | | | membrane protein, | | |
| | | | | 34 kDa), member 17; | | |
| | | | | solute carrier family | | |
| | | | | 25 (mitochondrial | | |
| | | | | carrier; peroxisomal | | |
| | | | | membrane protein, | | |
| | | | | 34 kDa), member 17 | | |
| 201299_s_at | 55233 | 5.10868443 | 0.02380637 | MOB1, Mps One | MOBK1B | — |
| | | | | Binder kinase | | |
| | | | | activator-like 1B | | |
| | | | | (yeast) | | |
| 214416_at | | 5.04452223 | 0.02470392 | — | — | — |
| 1556329_a_at | | 4.44152901 | 0.03507483 | Protocadherin 10 | PCDH10 | cell adhesion; |
| | | | | | | homophilic cell |
| | | | | | | adhesion |
| 1552892_at | 115650 | 4.42426086 | 0.03543146 | tumor necrosis factor | TNFRSF13C | immune response |
| | | | | receptor superfamily, | | |
| | | | | member 13C | | |
| 1557385_at | 84140 | 4.20114125 | 0.04039679 | hypothetical protein | FLJ13305 | — |
| | | | | FLJ13305 | | |
| 208035_at | 2916 | 4.16503582 | 0.04126654 | glutamate receptor, | GRM6 | regulation of |
| | | | | metabotropic 6 | | transcription, |
| | | | | | | DNA-dependent; |
| | | | | | | signal |
| | | | | | | transduction; |
| | | | | | | metabotropic |
| | | | | | | glutamate receptor |
| | | | | | | signaling pathway; |
| | | | | | | detection of visible |
| | | | | | | light; response to |
| | | | | | | stimulus |
| 243544_at | 125 | 4.10448367 | 0.04276965 | Alcohol | ADH1C | alcohol |
| | | | | dehydrogenase 1C | | metabolism; |
| | | | | (class I), gamma | | ethanol oxidation |
| | | | | polypeptide | | |
| 242514_at | | 3.52930644 | 0.04029301 | Guanine nucleotide | GNA12 | signal |
| | | | | binding protein (G | | transduction; G- |
| | | | | protein) alpha 12 | | protein coupled |
| | | | | | | receptor protein |
| | | | | | | signaling pathway; |
| | | | | | | G-protein coupled |
| | | | | | | receptor protein |
| | | | | | | signaling pathway; |
| | | | | | | blood coagulation |
| 232335_at | | 3.39842372 | 0.04525877 | CDNA FLJ33709 fis, | — | — |
| | | | | clone | | |
| | | | | BRAWH2007890 | | |
| 221288_at | 2845 | 2.91678586 | 0.04666233 | G protein-coupled | GPR22 | signal |
| | | | | receptor 22 | | transduction; G- |
| | | | | | | protein coupled |
| | | | | | | receptor protein |
| | | | | | | signaling pathway; |
| | | | | | | G-protein coupled |
| | | | | | | receptor protein |
| | | | | | | signaling pathway |
| 242115_at | | 2.56859018 | 0.04760049 | Solute carrier family | SLC39A9 | metal ion transport |
| | | | | 39 (zinc transporter), | | |
| | | | | member 9 | | |
| 1569557_at | 57209 | 2.33320192 | 0.04764119 | Zinc finger protein | ZNF248 | transcription; |
| | | | | 248 | | regulation of |
| | | | | | | transcription, |
| | | | | | | DNA-dependent |
| 1564544_x_at | | 2.23278224 | 0.04761104 | hypothetical protein | LOC644450 | — |
| | | | | LOC644450 | | |
| 228530_at | | 2.2203141 | 0.04762054 | Similar to RIKEN | RP11- | — |
| | | | | cDNA 2410129H14 | 11C5.2 | |

The performance of the prediction analysis was visualized by hierarchical clustering, which demonstrated the ability of the top scoring genes (Cox hazard ratio >10) to cluster the 53 specimens according to survival. FIG. 1A includes a table of the genes with a Cox score >10. On the array, MAGP2 and synaptotagmin-like 2 were measured by 3 and 2 probe sets, respectively. All of the probe sets yielded a significant Cox score; however, only the probe sets with the highest Cox score are presented in the table. As detailed in FIG. 1A, the gene possessing the highest hazard ratio was MAGP2.

To evaluate the accuracy of the prediction, a Kaplan-Meier plot was generated with the 53 samples equally divided into low or high-risk groups using the median predicted hazard as the cutoff (see FIG. 1B). The validity of the entire 200 probe set classifier was evaluated by a non-parametric log rank test using median hazard to stratify the patients. The test was highly significant (P=0.0029), with the high-risk group, defined by predicted hazard>median hazard, having a significantly shorter survival than the low-risk group (FIG. 1B). This result confirmed that the disclosed model was able to predict patient's hazard accurately. Other predictor compositions were also investigated by varying the number (np=100 or 300) of probe sets and PCs (n=4 or 6) that were assessed. Nearly identical results as those revealed with the initial parameters were observed.

To further characterize the survival signature, the 11 genes possessing the highest Cox hazard ratios were selected (see Table 2).

TABLE 2
Genes possessing the highest Cox hazard ratios.

| | | | | | | Biological |
|---|---|---|---|---|---|---|
| | | | | | Gene | Process/ |
| Probe Set | Locuslink | Cox score | p-value | Gene Title | Symbol | Description |
| 209758_s_at | 8076 | 30.3422259 | 3.62156E−08 | microfibril associated | MAGP2 | motility |
| | | | | glycoprotein 2 | | |
| 214043_at | 5789 | 20.82280417 | 5.03795E−06 | protein tyrosine | PTPRD | protein amino acid |
| | | | | phosphatase, receptor | | dephosphorylation; |
| | | | | type, D | | phosphate |
| | | | | | | metabolism; |
| | | | | | | transmembrane |
| | | | | | | receptor protein |
| | | | | | | tyrosine |
| | | | | | | phosphatase |
| | | | | | | signaling pathway; |
| | | | | | | protein amino acid |
| | | | | | | dephosphorylation |
| 235708_at | 152831 | 19.24436546 | 1.15009E−05 | klotho beta | KLB | carbohydrate |
| | | | | | | metabolism |
| 213943_at | 7291 | 18.57739807 | 1.63143E−05 | twist homolog 1 | TWIST1 | negative regulation |
| | | | | (acrocephalosyndactyly | | of transcription |
| | | | | 3; Saethre-Chotzen | | from RNA |
| | | | | syndrome) | | polymerase II |
| | | | | (Drosophila) | | promoter; skeletal |
| | | | | | | development; |
| | | | | | | regulation of |
| | | | | | | transcription, |
| | | | | | | DNA-dependent; |
| | | | | | | chromosome |
| | | | | | | organization and |
| | | | | | | biogenesis (sensu |
| | | | | | | Eukaryota); |
| | | | | | | morphogenesis; |
| | | | | | | cell differentiation; |
| | | | | | | transcription; |
| | | | | | | development; |
| | | | | | | regulation of |
| | | | | | | transcription |
| 205959_at | 4322 | 17.52838933 | 2.8305E−05 | matrix | MMP13 | peptidoglycan |
| | | | | metallopeptidase 13 | | metabolism; |
| | | | | (collagenase 3); matrix | | proteolysis; |
| | | | | metallopeptidase 13 | | proteolysis; |
| | | | | (collagenase 3) | | collagen |
| | | | | | | catabolism |
| 203439_s_at | 8614 | 17.45291968 | 2.94512E−05 | stanniocalcin 2 | STC2 | cell surface |
| | | | | | | receptor linked |
| | | | | | | signal |
| | | | | | | transduction; cell- |
| | | | | | | cell signaling; |
| | | | | | | response to |
| | | | | | | nutrient |
| 213125_at | 25903 | 17.35534771 | 3.10025E−05 | olfactomedin-like 2B | OLFML2B | — |
| 220351_at | 51554 | 17.27485333 | 3.23439E−05 | chemokine (C-C motif) | CCRL1 | chemotaxis; |
| | | | | receptor-like 1 | | immune response; |
| | | | | | | signal |
| | | | | | | transduction; G- |
| | | | | | | protein coupled |
| | | | | | | receptor protein |
| | | | | | | signaling pathway; |
| | | | | | | G-protein coupled |
| | | | | | | receptor protein |
| | | | | | | signaling pathway |
| 229088_at | | 16.46113525 | 4.96577E−05 | — | — | — |
| 210135_s_at | 6474 | 16.42717892 | 5.05551E−05 | short stature homeobox 2 | SHOX2 | skeletal |
| | | | | | | development; |
| | | | | | | regulation of |
| | | | | | | transcription, |
| | | | | | | DNA-dependent; |
| | | | | | | development; |
| | | | | | | nervous system |
| | | | | | | development; heart |
| | | | | | | development; |
| | | | | | | regulation of |
| | | | | | | transcription |
| 229404_at | 117581 | 16.33029866 | 5.32064E−05 | twist homolog 2 | TWIST2 | cell differentiation; |
| | | | | (Drosophila) | | negative regulation |
| | | | | | | of osteoblast |
| | | | | | | differentiation; |
| | | | | | | negative regulation |
| | | | | | | of transcription, |
| | | | | | | DNA-dependent; |
| | | | | | | regulation of |
| | | | | | | transcription; |
| | | | | | | transcription; |
| | | | | | | regulation of |
| | | | | | | transcription, |
| | | | | | | DNA-dependent; |
| | | | | | | development |

Quantitative RT-PCR was performed on all 53 RNA samples, which were included in the microarray analysis, using primers specific for each gene. Assayable expression levels for each gene were obtained in 49/53 samples, and then used to generate a Kaplan-Meier plot in a fashion analogous to the microarray analysis. Patients were divided into two groups with elevated or alleviated hazard according to the median predicted hazard. A non-parametric log rank test showed the two groups retained a significant (P=0.0107) survival difference (FIG. 1C) confirming the microarray data. These studies reveal a gene expression profile that can be used to predict the clinical outcome of a subject with ovarian cancer.

### Example 2

### Identification of Signaling Events Affecting Subject Survival

This example illustrates putative signaling events that contribute to subject survival.

To identify co-regulated pathways contributing to patient survival, PathwayStudio Version 4.0 software (Ariadne Genomics, Rockville, Md.) was used. This software package contains over 1 million documented protein interactions acquired from PubMed using the natural language processing algorithm MEDSCAN. The proprietary database can be used to develop a biological association network (BAN) to identify putative signaling pathways. By overlaying expression data over the BAN as well as survival associated gene identities, co-regulated genes defining specific signaling pathways were identified.

To ascertain whether subsets of the survival associated genes participate in coordinated signaling pathway(s) contributing to patient outcome, the 53 advanced ovarian tumor specimens were compared to 10 normal ovarian surface epithelium brushings analyzed with the identical AFFYMETRIX® array platform. A total of 5022 probe sets were differentially regulated (P<0.001) in the tumor isolates with a fold change≧±1.5 and a 90% confidence the data set contained no more than 5% false discoveries. A biological association network (BAN) was constructed from the gene list with PathwayStudio 4.0 software. Both differential gene expression data and identifiers for the top 200 survival associated probe sets were overlaid onto the BAN to identify co-regulated pathways.

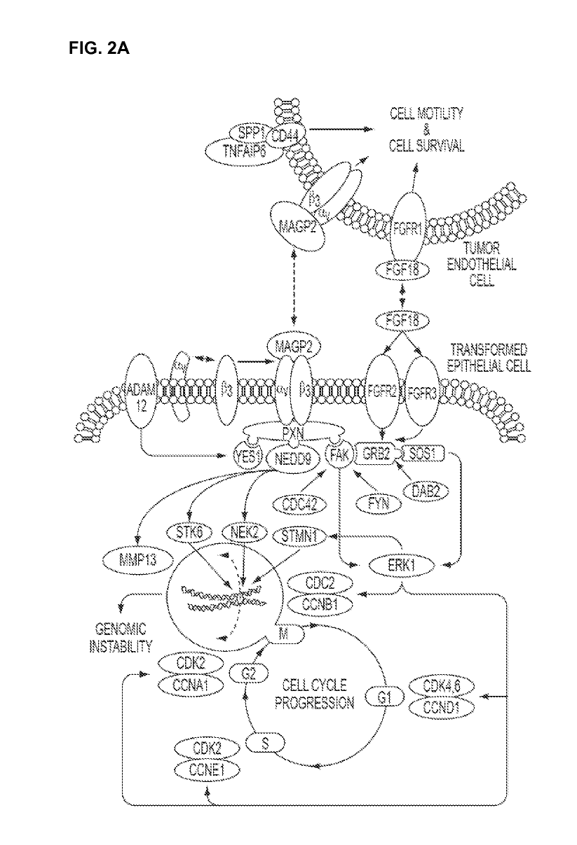

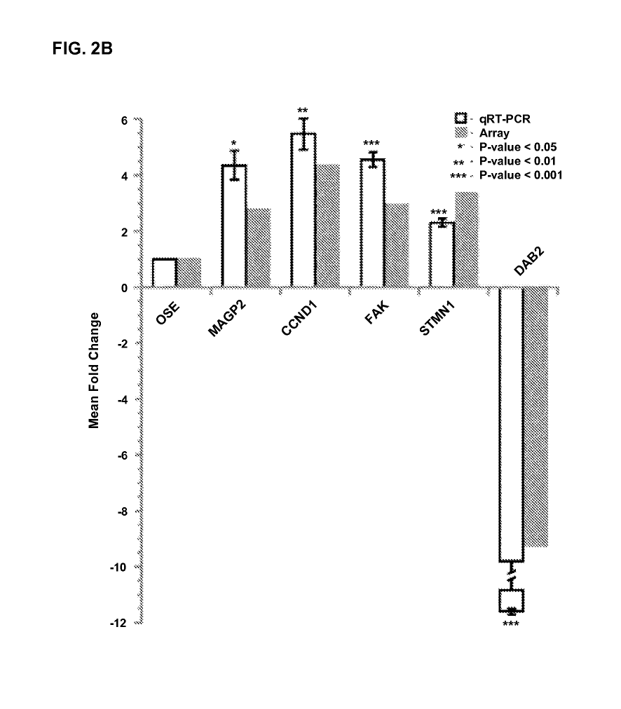

Integrin mediated signaling stimulated by MAGP2 engagement of the αᵥβ₃ receptor featured prominently in the analysis (FIG. 2A). While the receptor subunits were not differentially regulated in the tumor specimens, a number of downstream effectors were over-expressed versus OSE including PXN, FAK, GRB2, and SOS1. Subsequent ERK1 induction can contribute to increased cell cycle progression and increased chromosomal instability. Interestingly, CDC42 and FYN, which are both FAK regulators implicated in cell polarity and motility, were down-regulated, indicating that pro-survival signaling is the principal endpoint of this pathway.

Contributing to this pathway were a number of genes implicated in patient survival including MAGP2, FGF18, FGFR2, ADAM12, NEDD9, MMP13, and CDC2. Of these, MAGP2, FGF18, FGFR2, and CDC 2 were also significantly upregulated, when compared to OSE. As with integrin receptor signaling, FGF receptor engagement can also activate ERK via GRB2/SOS1. NEDD9 has been associated with increased genomic instability through its ability to induce STK6 and NEK2. In addition, NEDD9 can increase expression of MMP13, which was also identified as a survival associated transcript. A unique feature of MAGP2, FGF18, and TNFAIP6 is their ability to modulate endothelial cell behavior. The cognate receptors of each protein are also expressed in tumor endothelial cells indicating that patient survival may consist of signaling events specific to the transformed cell, as well as induced endothelial cell changes. Furthermore, while these pathways were upregulated on average across all of the tumor specimens, as compared to normal OSE, it is possible that pronounced expression of one or more survival associated genes may dramatically enhance the aggressiveness of the disease negatively impacting patient outcome.

To substantiate the pathway analysis, qRT-PCR was completed for 5 differentially regulated genes including MAGP2, CCND1, FAK, STMN1, and DAB2. Quantitative RT-PCR was performed as described in Example 1. Relative expression levels were calculated according to the 2^{-ΔΔC}_{T} method using C_{T} values determined for all 53 tumor specimens, as well as the 10 normal OSE isolates. The expression data was normalized to the average of three housekeeping genes (GUSB, GAPDH, and Cyclophillin). A student's t-test confirmed all 5 genes were differentially regulated relative to normal OSE at levels comparable to the array data (FIG. 2B).

### Example 3

### Characterization of Clinical Correlates Associated with the Survival Signature Gene MAGP2

This example characterizes clinical correlates associated with the survival signature gene MAGP2.

While the probe sets identified in the analysis predicts patient survival as a group, each gene was selected according to its individual Cox hazard ratio. Thus, genes possessing a high hazard ratio may independently predict for patient survival. MAGP2 was identified by 3 separate probe sets and scored the highest hazard ratio. In addition, pathway analysis indicated it might participate in co-regulated signaling events contributing to enhanced tumor cell survival and prolonged endothelial cell survival and motility. The combination of a clear clinical correlation with putative biological consequences in two cell types distinguished MAGP2 as a candidate for further characterization.

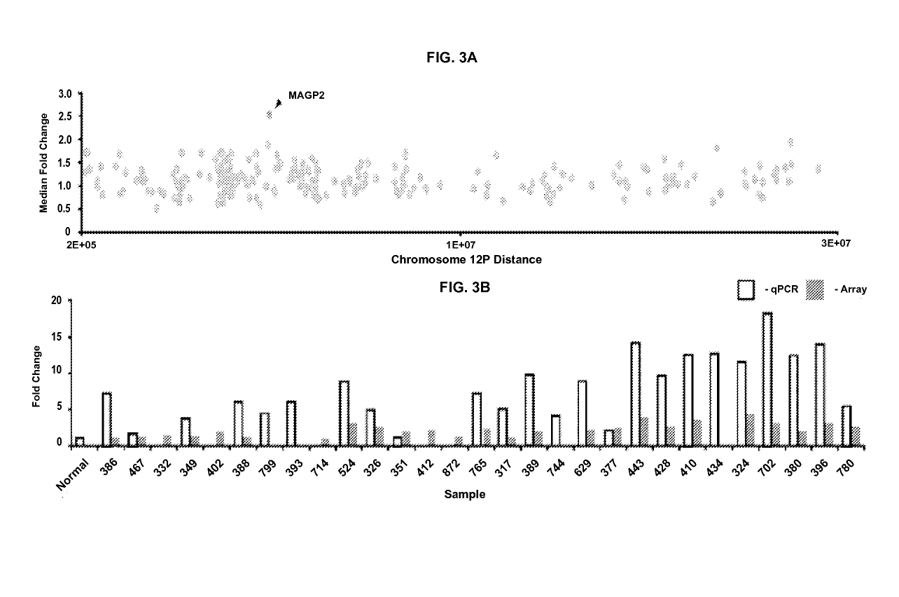

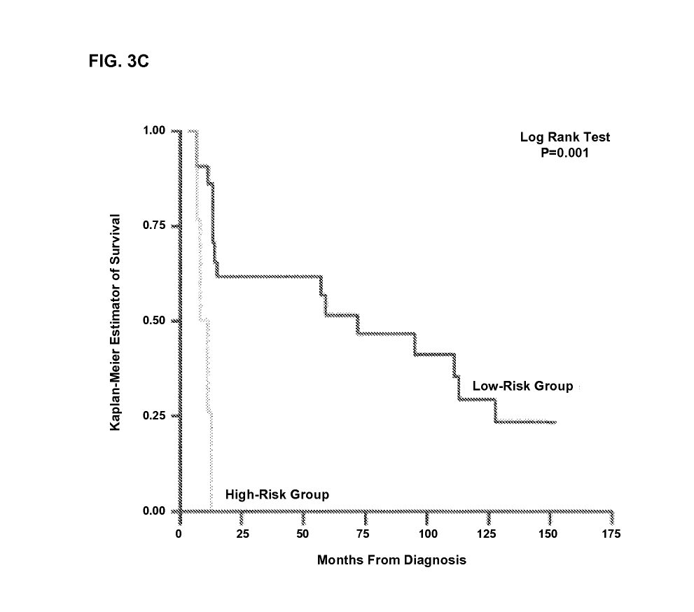

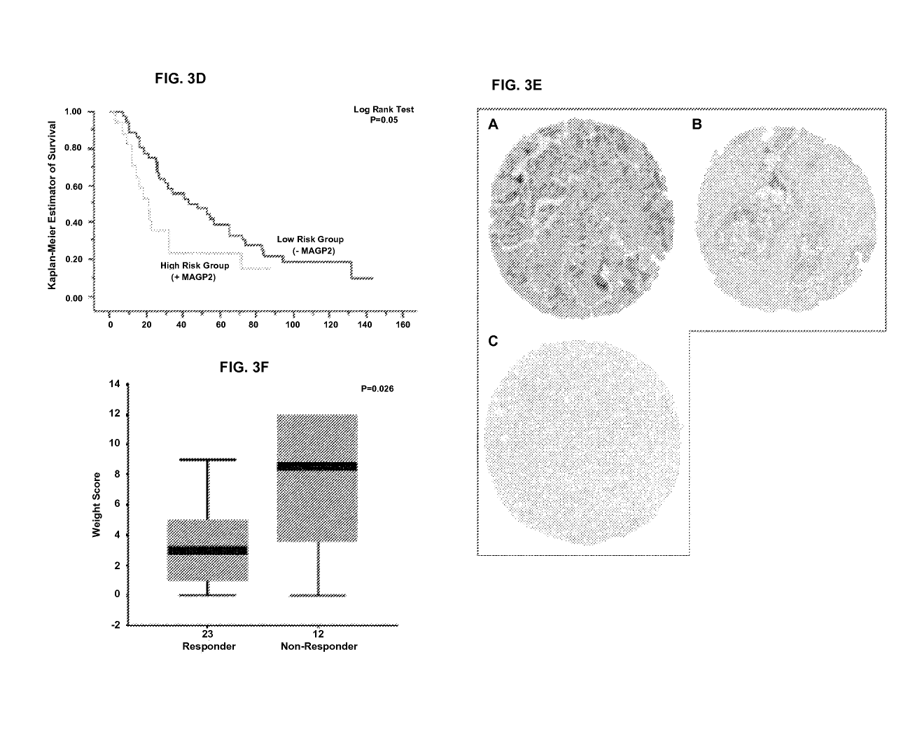

MAGP2 was evaluated as an independent prognostic factor. Tumor cells from 42 late stage, high grade serous adenocarcinomas were procured by laser based microdissection. DNA was extracted, amplified, labeled, and hybridized onto a 60-mer 22K oligonucleotide array platform overnight at 42° C. for comparative genome hybridization analysis. Scanning and signal quantification were performed followed by sample normalization to identify amplified genomic regions. MAGP2 demonstrated a median copy number approaching 2.5 in a subset of the tumor specimens indicating the locus is abnormally amplified in ovarian cancer (FIG. 3A). This observation was supported by qPCR analysis correlating amplification of the locus with mRNA expression values (FIG. 3B). qRT-PCR analysis using all 53 tumor isolates confirmed the association between MAGP2 expression and patient survival. Stratifying the expression values according to the mean evidenced a significantly shorter survival time for patients expressing MAGP2 mRNA above the mean (FIG. 3C). Immunolocalization of MAGP2 in all 53 optimally debulked stage III grade 3 serous adenocarcinoma, as well as normal ovarian epithelium and benign cysts, demonstrated low-level expression of MAGP2 in normal ovarian epithelial cells and benign cysts, but elevated levels in a proportion of malignant tumors. The intensity of MAGP2 staining was correlated with the survival data and examined by Kaplan Meier survival analysis. Statistical significance was determined by a log rank test. The results indicated that patients positive for MAGP2 expression (>mean+1 S.D. weight score) possessed a poor prognosis (P=0.05) (FIG. 3D). Independent validation of this finding was completed by correlating MAGP2 protein expression levels with overall survival across an 81 specimen tissue microarray (TMA). A broad range of staining intensities was observed across the tumor sections (FIG. 3E, subpanel A, high-level staining; FIG. 3E, subpanel B, moderate staining; FIG. 3E, subpanel C, low-level staining) Cox regression analysis demonstrated that elevated MAGP2 protein expression, adjusted for debulking status, showed a significant association with poor patient outcome (Cox score: 1.857; 95% CI (1.253, 2.752), p=0.004, age adjusted)

A subset (35) of the 53 tumors analyzed were evaluated for chemotherapeutic response status. Tumors were stratified into two groups, resistant and responsive to chemotherapy, as defined by objective evidence of complete or partial remission. MAGP2 expression levels were significantly lower in patients who responded to chemotherapy (P=0.008) (FIG. 3F). These data indicate that MAGP2 can play a role in a subject's response to chemotherapy.

### Example 4

### Recombinant MAGP2 Stimulates Serous Ovarian Cancer Cell Adhesion and Survival

This example illustrates that recMAGP2 stimulates serous ovarian cancer cell adhesion and survival.

Synthesis of recombinant MAGP2. A full-length cDNA for MAGP2 was generated and cloned into a pcDNA3 mammalian expression vector. To evaluate the biological activity of the protein, the construct was transfected into 293T cells. The supernatant was evaluated by western blot to confirm MAGP2 was secreted and expressed at the correct size. Post transfection supernatants were then tested as a chemo-attractant in HUVEC motility assays. The validated construct was then subcloned into a PICZaplhaA inducible yeast expression vector, transformed into competent yeast, and induced. Secreted recombinant protein was harvested from the yeast supernatant, purified, and used for downstream in vitro biological assays in ovarian and endothelial cell lines.

Cell Lines and Culture Conditions. A224, UCI107 and OVCA429 ovarian cancer cell lines were maintained in RPMI (Invitrogen Life Technologies Inc, Carlsbad, Calif.) supplemented with 10% fetal bovine serum (Gemini Bio-Products, Woodland, Calif.) and 1% L-glutamine (Invitrogen Life Technologies).

Western Blot Analysis. Cell lysates from the ovarian cancer cell lines were prepared by lysing the cells in RIPA buffer (150 mM NaCl, 1% Triton X-100, 1% deoxycholate, 0.1% SDS, 10 mM Tris pH7.4) supplemented with 0.1 mM phenylmethylsulfonyl fluoride (PMSF), 100 μg/ml aprotinin, 100 μg/ml leupeptin and 1 mM sodium orthovanadate. The cell lysates were briefly sonicated and centrifuged to remove debris, and protein concentrations were determined using the BCA protein assay (Pierce Biotechnology, Inc., Rockford, Ill.). Equal amounts of protein were separated on 4-12% SDS gels, transferred to nitrocellulose membranes (Amersham Biosciences, Piscataway, N.J.), and incubated with an anti-MAGP2 antibody (Rockland Inc., Gilbertsville, Pa.). The signal was detected by enhanced chemiluminescence.

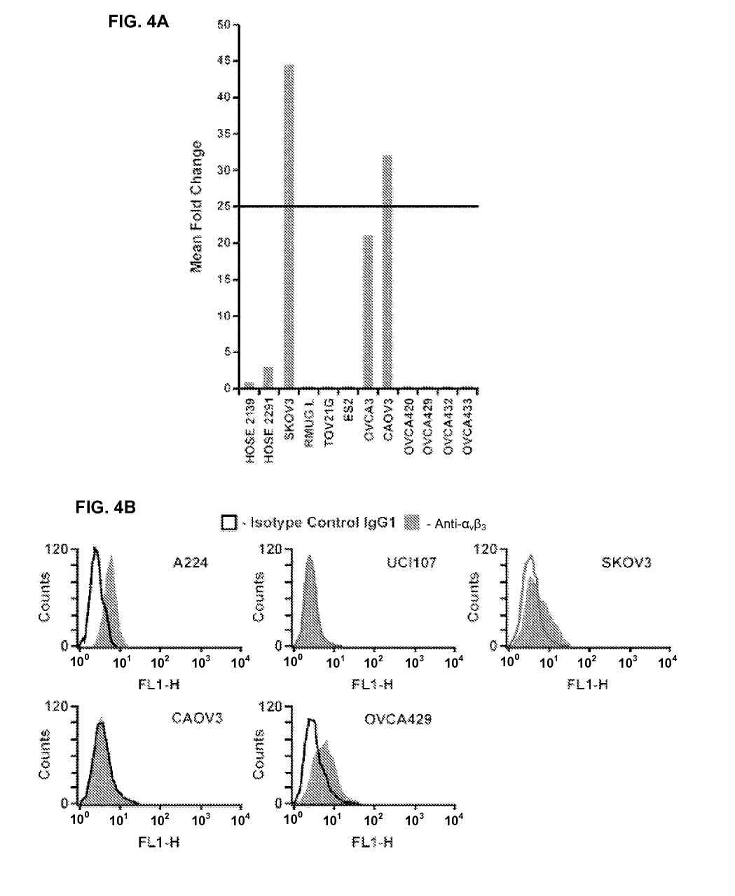

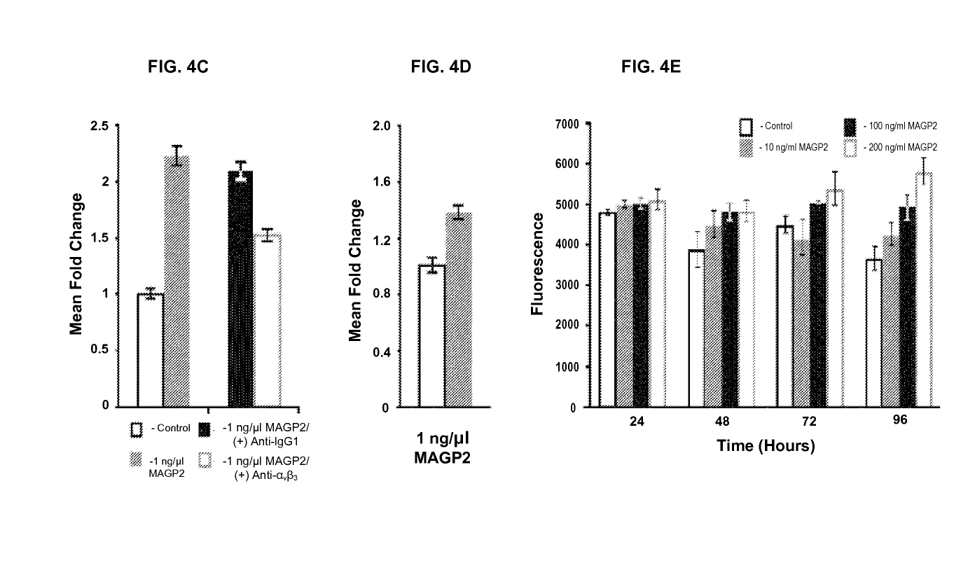

To demonstrate that MAGP2 can modulate tumor cell biology, recombinant MAGP2 (recMAGP2) was synthesized. Since MAGP2 has been shown to induce adhesion in a number of different cell types via the αᵥβ₃ integrin receptor (Gibson et al., J. Biol. Chem. 271: 1096-1103, 1999), this endpoint was selected to verify the biological activity of the construct. Screening a serous ovarian cancer cell line panel for MAGP2 expression by qRT-PCR (FIG. 4A) and western blot, as well as αᵥβ₃ receptor status through FACs analysis (FIG. 4B, select cell lines only), facilitated the selection of cell lines amenable for downstream examination. Quantitative RT-PCR was performed as described in Example 1. Among cell lines expressing MAGP2, only the SKOV3 cell line co-expressed the αᵥβ₃ receptor. Two cell lines, A224 and OVCA429, were positive for αᵥβ₃, yet lacked measurable levels of MAGP2. These cell lines were chosen for subsequent analyses involving the recombinant protein along with UCI107, which was negative for both the receptor and MAGP2.

The effect of recMAGP2 on adhesion was evaluated for A224 and UCI107 cell lines. When plated on recMAGP2 coated wells, A224 cells displayed an increase in adhesion (P<0.0005) (FIG. 4C), relative to control wells. Pre-incubation of A224 cells with anti-αᵥβ₃ integrin blocking antibody resulted in a decrease (P<0.005) in adhesion on recMAGP2 coated wells (FIG. 4C), while pre-treatment with control IgG1 antibody had no effect. In contrast, UCI107 showed only a modest increase in adhesion at high concentrations of recMAGP2 further demonstrating the suitability of recMAGP2 for biological evaluation (FIG. 4D).

Based on the pathway analysis, αᵥβ₃ mediated signaling was implicated in ovarian tumor cell survival through the stimulation of critical cell-cycle checkpoint regulators. To assess this observation, OVCA429 cells were cultured under increasing concentrations of purified recMAGP2 and harvested at 24-hour intervals. At 96 hours, a significant difference in survival was observed for cells treated with 200 ng/ml of recMAGP2 (P=<0.01) (FIG. 4E). This observation substantiated the pathway indicating that differential patient survival can be attributed in part to enhanced tumor cell survival through MAGP2 induced signaling events.

### Example 5

### Recombinant MAGP2 Stimulates the Migration, Invasion, and Survival of Endothelial Cells

This example illustrates that recMAGP2 stimulates the migration, invasion and survival of HUVE cells.

Cell Lines and Culture Conditions. HUVE cells were maintained in DMEM (Invitrogen Life Technologies Inc, Carlsbad, Calif.) supplemented with 10% fetal bovine serum (Gemini Bio-Products, Woodland, Calif.) and 1% L-glutamine (Invitrogen Life Technologies).

Beyond prolonging tumor cell survival, MAGP2 could also stimulate endothelial cell survival and motility. As indicated in FIG. 2A, secreted MAGP2 may modulate the biology of surrounding endothelial cells ultimately promoting tumor angiogenesis. To demonstrate these biological effects, human umbilical vein endothelial (HUVE) cells were cultured in the presence of recMAGP2.

Cell motility was evaluated for HUVE cells (5×10⁴) by adding them to the top chamber of a BD Falcon HTS FluoroBlok insert with a PET membrane with eight mm pore (BD Biosciences) in 200 ml of medium with 0.2% FBS. The inserts were placed into the bottom chamber wells of a 24-well plate containing medium with increasing concentrations of recombinant MAGP2. After 2.5 hr, cells that migrated through the pores of the membrane to the bottom chamber were stained with calcein 8 mg/ml (Molecular Probes, Eugene, Oreg.) in PBS for 30 min at 37° C. The fluorescence of migrated cells was quantified using a fluorometer set at 485 nm excitation and 530 nm emission. Parallel control experiments were performed using the MAGP2 elution buffer. HUVECs motility was mediated through αᵥβ₃ integrin, cells were pre-treated either with an anti-αᵥβ₃ integrin antibody (Santa Cruz Laboratories #sc-7312) or with an equal amount of IgG (ICL #RS-90G1) for 30 minutes before the addition of 100 ng/ml MAGP2. The motility assay was then performed as described above.

HUVE cell invasion was measured by adding cells (5×10⁴) to the top chamber of a BD Falcon HTS FluoroBlok insert with a PET membrane coated with a thin layer of Matrigel. To evaluate the effect of MAGP2 on HUVE cell proliferation, 1,000 HUVECs/well were plated in 8 wells of a 96-well plate in EBM2 with 2.0% FBS. The next day the media were removed and replaced with 0.2% FBS media with elute buffer control or with recombinant MAGP2 protein at 4.5 ng/ml. After 2 days, elute control or MAGP2 was added a second time. Cell number was quantified via MTT assay.

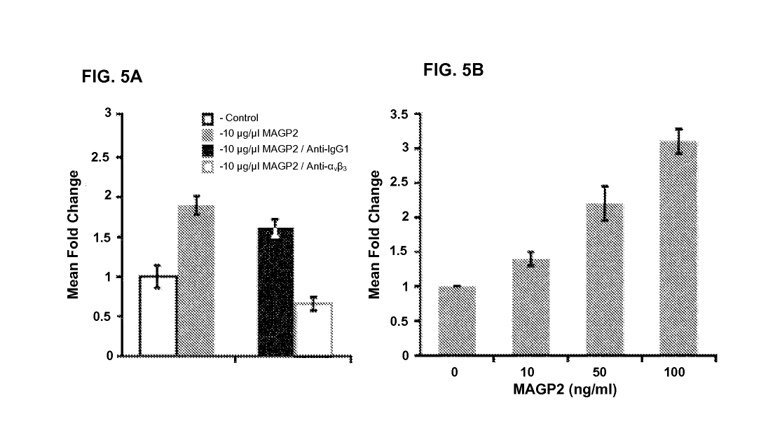

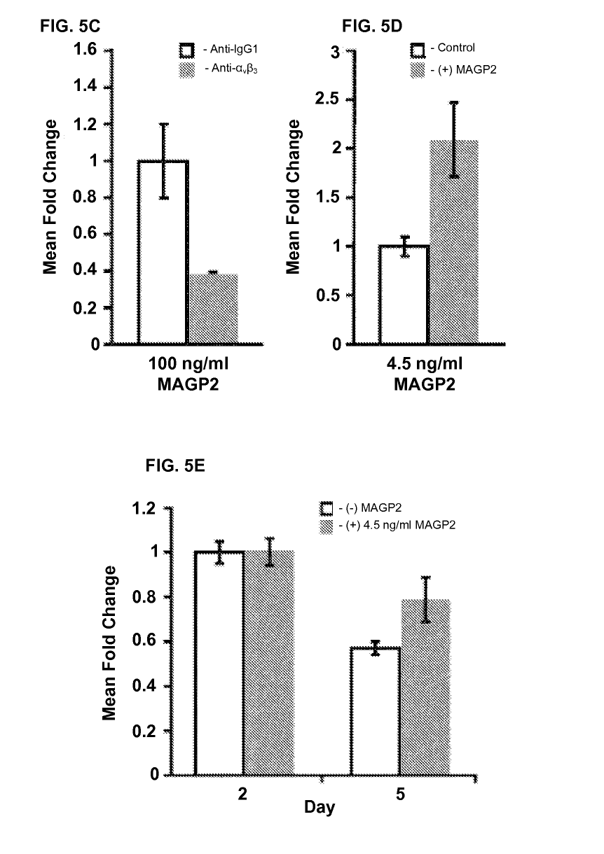

As observed for A224 ovarian cancer cells, HUVE cells plated on recMAGP2 showed a 2.5 fold increase in adhesion (P<0.005) (FIG. 5A). This effect was ablated by pre-treatment with a αᵥβ₃ integrin blocking antibody (P<0.005), whereas control IgG1 antibody did not reduce adhesion (FIG. 5A). Thus, recMAGP2 also exerts its biological activity through the αᵥβ₃ integrin receptor in HUVE cells.

To determine the effect of recMAGP2 on the HUVE cell motility, recMAGP2 was used as a chemoattractant. After 2.5 hours, recMAGP2 increased the motility of the cells in a dose dependent manner (P<0.05), as compared to cells incubated with medium alone (FIG. 5B). The addition of anti-αᵥβ₃ antibody in the presence of recMAGP2 attenuated cell motility (FIG. 5C). Similarly, exposing HUVE cells seeded onto Matrigel matrix to reMAGP2 stimulated a 2-fold increase in invasion (P<0.05) (FIG. 5D).

In addition to motility and invasion, HUVE cell survival under low serum conditions was prolonged by recMAGP2. Cells grown in the absence of recMAGP2 in low serum for 5 days displayed a reduction in survival (P<0.005), when compared to cells grown in the presence of recombinant protein (FIG. 5E). Summation of these results indicates MAGP2 is a potent modulator of endothelial cell behavior, which may contribute to angiogenesis and patient survival.

### Example 6

### Identification of Signaling Events Contributing to the Effect of Recombinant MAGP2 on HUVE Cells

This example elucidates the signaling events that contribute to the effect of recombinant MAGP2 on HUVE cells.

Western Blot Analysis. To assess FAK phosphorylation in HUVE cells, cells were plated on 60 mm² dishes treated with 100 ng/ml MAGP2 recombinant protein or elute buffer control. After 30 minutes the cells were washed with 4° C. PBS and lysed in RIPA buffer containing 0.1% SDS and proteinase inhibitors. Western blotting was then performed with 5 μg of protein under denaturing conditions on a 4-12% NuPAGE gel and transferred to a PVDF membrane. The membrane was blocked in 5% nonfat milk for 1 hr at RT, probed with a 1:1000 dilution of anti-FAK[pY⁴⁰⁷] rabbit polyclonal 1° antibody (Biosource) for 2 hr at RT. The blot was washed 3 times for 5 min with TBST and probed with a 1:2000 dilution of goat anti-rabbit HRP conjugated 2° antibody (Amersham) for 1 hr at RT. The blot was washed 3 times for 10 min with TBST and bands were detected via chemiluminscence using an ECL kit (Amersham).

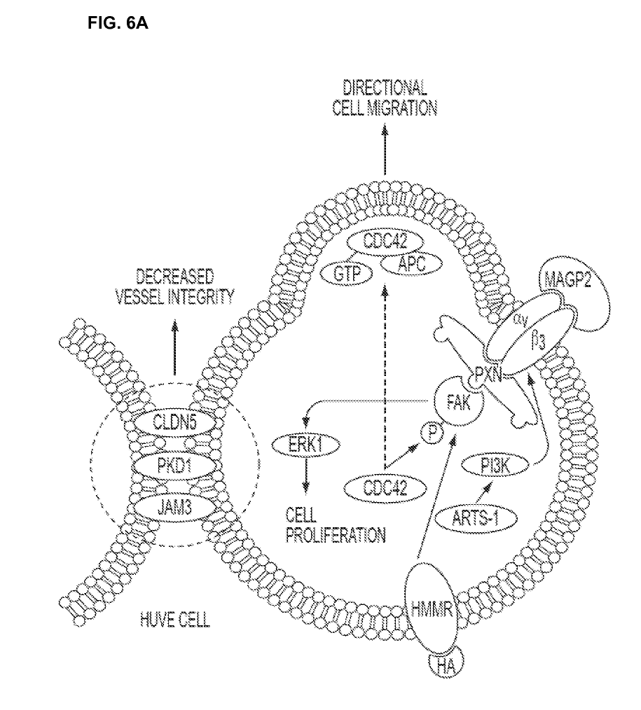

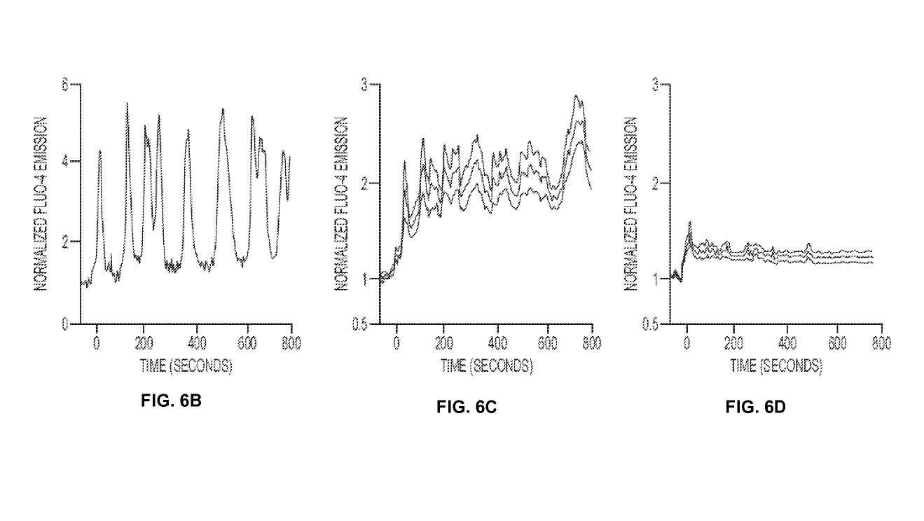

The marked effect of recMAGP2 on HUVE cell behavior indicated a change in cellular regulation had taken place possibly affecting gene expression. To explore the consequences of recMAGP2 induced signaling, Affymetrix® U133 Plus 2.0 microarrays were completed for a series of recMAGP2 treated (n=3) and untreated (n=3) HUVE cell isolates. A total of 274 probe sets were identified for pathway analysis in cells exposed to recMAGP2 (P<0.01 and fold-change ≧1.5) (FIG. 6A). Among them were αᵥ integrin, FAK, and CDC42 indicating that MAGP2 stimulated signaling may induce the expression of key effectors enhancing its activity in HUVE cells. In addition, a number of genes involved in the formation of endothelial cell tight junctions including CLDN5, PKD1, and JAM3 were down regulated, while motility genes CDC42 and APC were upregulated. Deregulation of these genes may lead to a reduction in vessel integrity permitting endothelial cell migration and neovascularization.

Since FAK is a mediator of αᵥβ₃ signaling, the phosphorylation status of FAK in recMAGP2 treated cells was confirmed. Using a phosphorylation specific antibody recognizing tyrosine 407, a western blot comparing phospho-FAK to total FAK was completed for both treated and untreated HUVE cell isolates. In cells treated with the recombinant protein, there was an increase in phosphorylated FAK indicating elevated levels of activated protein, while no change was evidenced in untreated cells.

To identify the mechanism underlying the stimulation of FAK by recMAGP2, Ca²⁺ oscillation was assessed in treated HUVE cells. Cells cultured in recombinant protein were loaded with fluo-4/AM and monitored with a confocal microscope. recMAGP2 induced an immediate increase in [Ca²⁺]_{I} levels which oscillated at a sustained frequency of 100 s/cycle in individual cells (FIGS. 6B and 6C). When cells were pre-treated with a synthetic RGD peptide, the effect of recMAGP2 on [Ca²⁺]_{I} levels was severely diminished (FIG. 6D). These data indicate that FAK is activated through Ca²⁺ mobilization in response to recMAGP2 in HUVE cells. This may account in part for the enhanced motility and survival of HUVE cells as described in the pathway analysis.

### Example 7

### MAGP2 Expression is Significantly Correlated with CD34 Expression in Serous Ovarian Cancer

This example illustrates a role for MAGP2 in inducing angiogenesis in ovarian tumors.

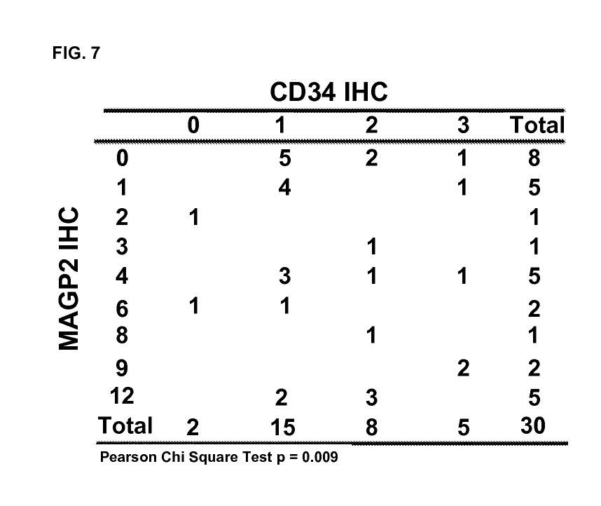

Given the observed pronounced biologic effect of MAGP2 on endothelial cells, it was proposed that tumors with elevated MAGP2 expression would display features indicative of a pro-angiogenic microenvironment. To evaluate whether MAGP2 induces angiogenesis in ovarian tumors, MAGP2 expression was correlated with microvessel density in 30 advanced serous cancers. MAGP2 expression was determined by immunolocalization of the MAGP2 protein using an anti-MAGP2 polyclonal antibody, while microvessel density was assayed for by immunolocalization of CD34 positive microvasculature within the tissue using an anti-CD34 monoclonal antibody. Microvessel density was evaluated based on number of CD34 positive microvessels within the tumor section (scale 0-3). MAGP2 staining was scored based on intensity and the percentage of positive cells (weight score 0-12). A Pearson Chi Square test demonstrated a significant correlation between MAGP2 expression and CD34 positive microvessel density (P=0.009) (FIG. 7). This data indicates that MAGP2 plays a role in ovarian tumor neovascularization.

### Example 8

### Treatment of an Ovarian Tumor in a Human

This example describes a particular method that can be used to treat a primary or metastatic ovarian tumor in humans by administration of one or more agents that inhibit or reduce the biological activity (such as expression) of one or more of the disclosed ovarian survival factor-associated molecules that are upregulated in an ovarian tumor. Although particular methods, dosages, and modes of administrations are provided, one skilled in the art will appreciate that variations can be made without substantially affecting the treatment.

Based upon the teaching disclosed herein, an ovarian tumor, such as ovarian cancer can be treated by administering a therapeutically effective amount of a composition, wherein the composition comprises an agent (such as a specific binding agent) that modulates the biological activity of one or more ovarian survival factor-associated molecules provided in Tables 1 or 2 (such as, MAGP2), thereby reducing or eliminating the activity of the one or more ovarian survival factor-associated molecules (such as, inhibiting the expression or biological activity of MAGP2) which in turn increases the response of the tumor to therapeutic agents (such as, chemotherapy).

Briefly, the method can include screening subjects to determine if they have an ovarian tumor, such as advanced ovarian cancer. Subjects having ovarian cancer are selected. In one example, subjects having increased levels of one or more of the disclosed ovarian survival factor-associated molecules in their serum are selected. In one example, a clinical trial would include half of the subjects following the established protocol for treatment of ovarian cancer (such as a normal chemotherapy/radiotherapy/surgery regimen). The other half would follow the established protocol for treatment of the tumor (such as a normal chemotherapy/radiotherapy/surgery regimen) in combination with administration of the therapeutic compositions described above. In some examples, the tumor is surgically excised (in whole or part) prior to treatment with the therapeutic compositions. In another example, a clinical trial would include half of the subjects following the established protocol for treatment of ovarian cancer (such as a normal chemotherapy/radiotherapy/surgery regimen). The other half would follow the administration of the therapeutic compositions described above. In some examples, the tumor is surgically excised (in whole or part) prior to treatment with the therapeutic compositions.

Screening subjects—In some examples, the subject is first screened to determine if they have ovarian cancer. In particular examples, the subject is screened to determine if the tumor is a grade one ovarian tumor, grade 2 ovarian tumor, grade 3 ovarian tumor or grade 4 ovarian tumor. Examples of methods that can be used to screening for ovarian cancer include a combination of ultrasound, tissue biopsy, and serum blood levels. If blood or a fraction thereof (such as serum) is used, 1-100 μA of blood is collected. Serum can either be used directly or fractionated using filter cut-offs to remove high molecular weight proteins. If desired, the serum can be frozen and thawed before use. If a tissue biopsy sample is used, 1-100 μg of tissue is obtained, for example using a fine needle aspirate.

In some examples, the biological sample (e.g., tissue biopsy or serum) is analyzed to determine if it overexpresses one or more of the disclosed ovarian survival factor-associated molecules listed in Table 1 or 2, such as MAGP2, wherein the presence of such overexpression indicates that the tumor can be treated with the disclosed therapies. For example, the disclosed gene profile can be used to determine if one or more of the ovarian survival factor-associated molecules is increased. In some examples, the biological sample is analyzed to determine if the subject has a grade 1 ovarian tumor, grade 2 ovarian tumor, grade 3 ovarian tumor, or grade 4 ovarian tumor.

In a specific example, epithelial tumor cells (approximately 5,000 tumor cells) are procured from the biological sample. RNA is isolated and purified from these cells using routine methods, such as using a commercial kit (e.g., an RNeasy Micro Kit according to the manufacturer's protocol; Qiagen; Valencia, Calif.). The purified RNA is then amplified and hybridized to a microarray including the disclosed ovarian survival factor-associated molecules. The increased expression (such as an increase of at least 2-fold, at least 3-fold, or at least 5-fold) of one or more of the disclosed ovarian survival factor-associated molecules, such as MAGP2, relative to control values (e.g., expression level in a subject without ovarian cancer) is indicative that the subject has a poor prognosis and is a candidate for receiving the therapeutic compositions disclosed herein. However, such pre-screening is not required prior to administration of the therapeutic compositions disclosed herein (such as those that include a specific binding agent that inhibits or reduces expression of one of the disclosed ovarian survival factor-associated molecules).

### Pre-Treatment of Subjects

In particular examples, the subject is treated prior to administration of a therapeutic composition that includes one or more agents to one or more of the disclosed ovarian survival factor-associated molecules. However, such pre-treatment is not always required, and can be determined by a skilled clinician. For example, the tumor can be surgically excised (in total or in part) prior to administration of one or more specific binding agents to one or more of the disclosed ovarian survival factor-associated molecules. In addition, the subject can be treated with an established protocol for treatment of the particular tumor present (such as a normal chemotherapy/radiotherapy regimen).

### Administration of Therapeutic Compositions

Following subject selection, a therapeutic effective dose of the composition including the agent is administered to the subject. For example, a therapeutic effective dose of an agent to one or more of the disclosed ovarian survival factor-associated molecules is administered to the subject to reduce or inhibit tumor growth and/or vascularization. Administration can be achieved by any method known in the art, such as oral administration, inhalation, or inoculation (such as intramuscular, ip, or subcutaneous). In some examples, the agent is a siRNA. In another example, the agent is an antibody. In a further example, the agent is conjugated to a therapeutic agent such as a cytotoxin, chemotherapeutic reagent, radionucleotide or a combination thereof.

The amount of the composition administered to prevent, reduce, inhibit, and/or treat ovarian cancer or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., ovarian cancer) in a subject without causing a substantial cytotoxic effect in the subject. An effective amount can be readily determined by one skilled in the art, for example using routine trials establishing dose response curves. In addition, particular exemplary dosages are provided above. The therapeutic compositions can be administered in a single dose delivery, via continuous delivery over an extended time period, in a repeated administration protocol (for example, by a, daily, weekly, or monthly repeated administration protocol). In one example, therapeutic compositions that include one or more siRNAs having 95% identity to the disclosed ovarian survival factor-associated molecules are administered iv to a human. As such, these compositions may be formulated with an inert diluent or with an pharmaceutically acceptable carrier.

In one specific example, siRNAs are administered according to the teachings of Soutschek et al. (Nature Vol. 432: 173-178, 2004), Karpilow et al. (Pharma Genomics 32-40, 2004) or as summarized by Aigner (J. Biotech. 124: 12-25, 2006). siRNAs can be administered by several administrative routes including intratumoral, intravenous, intraperitoneal, subcutaneous or intranasal depending upon the siRNA formulation. For example, siRNA molecules can be complexed with polyethylenimines to form polyethylenimine/siRNA complexes. These complexes can then be delivered in vivo by intraperitoneal or subcutaneous injection at 20 to 2000 nM final siRNA concentration and internalized by tumor cells within a few hours leading to the intracellular release of siRNA molecules, which display full bioactivity. In another specific example, naked antibodies are administered at 5 mg per kg every two weeks or 10 mg per kg every two weeks depending upon the stage of the ovarian cancer. In an example, the antibodies are administered continuously. In another example, antibodies or antibody fragments conjugated to cytotoxic agents (immunotoxins) are administered at 50 μg per kg given twice a week for 2 to 3 weeks.

Administration of the therapeutic compositions can be continued after chemotherapy and radiation therapy is stopped and can be taken long term (for example over a period of months or years).

### Assessment

Following the administration of one or more therapies, subjects having a tumor (for example ovarian cancer) can be monitored for tumor treatment, such as regression or reduction in metastatic lesions, tumor growth or vascularization. In particular examples, subjects are analyzed one or more times, starting 7 days following treatment. Subjects can be monitored using any method known in the art. For example, diagnostic imaging can be used (such as x-rays, CT scans, MRIs, ultrasound, fiber optic examination, and laparoscopic examination), as well as analysis of biological samples from the subject (for example analysis of blood, tissue biopsy, or other biological samples), such as analysis of the type of cells present, or analysis for a particular tumor marker. In one example, if the subject has advanced ovarian cancer, assessment can be made using ultrasound, MRI, or CAT scans, or analysis of the type of cells contained in a tissue biopsy. It is also contemplated that subjects can be monitored for the response of their tumor(s) to therapy during therapeutic treatment by at least the aforementioned methods.

### Additional Treatments

In particular examples, if subjects are stable or have a minor, mixed or partial response to treatment, they can be re-treated after re-evaluation with the same schedule and preparation of agents that they previously received for the desired amount of time, such as up to a year of total therapy. A partial response is a reduction in size or growth of some tumors, but an increase in others.

### Example 9

### Screening of Agents to Treat an Ovarian Tumor

This example describes methods that can be used to identify agents to treat an ovarian tumor.

According to the teachings herein, one or more agents for the use of treating an ovarian tumor, such as ovarian cancer, can be identified by contacting an a cell, such as an ovarian tumor epithelial cell, with one or more test agents under conditions sufficient for the one or more test agents to alter the activity of at least one ovarian survival factor-associated molecule listed in Table 1 or 2. The method can also include detecting the activity of the at least one ovarian survival factor-associated molecule in the presence and absence of the one or more test agents. The activity of the at least one ovarian survival factor-associated molecule in the presence of the one or more test agents is then compared to the activity in the absence of such agents to determine if there is decreased expression of the at least one ovarian survival factor-associated molecule. Decreased expression of the ovarian survival factor-associated molecule indicates that the one or more test agent is of use to treat the ovarian tumor. Decreased expression can be detected at the nucleic acid or protein level. An RNA expression product can be detected by a microarray or PCR by methods described above (see, for example, Example 1). A protein expression product can be detected by standard Western blot or immunoassay techniques that are known to one of skill in the art. However, the disclosure is not limited to particular methods of detection.

In a specific example, a library of natural products are obtained, for example from the Developmental Therapeutics Program NCI/NIH, and screened for their effect on the disclosed ovarian survival factor-associated molecules, for example by decreasing the expression of one or more of the disclosed ovarian survival factor-associated molecules, such as MAGP2, PTPRD, KLB, TWIST1 and MMP13.

Immortalized ovarian cancer cells, such as UCI107 and SKOV3 ovarian cancer cells, are combined with serial dilutions of each compound (1 nM to 10 mM).

The sample is incubated from between 10 minutes and 24 hours to assess the expression of one or more of the disclosed ovarian survival factor-associated molecules. The effect of the compound on the expression of one or more of the disclosed ovarian survival factor-associated molecules is determined by methods known in the art including microarray analysis or PCR. For example, the disclosed gene profile can be used to determine if a given compound is effective at treating an ovarian tumor. Alternatively, the cells are screened for decreases in ovarian survival factor-associated proteins by Western blot or other immunoassay techniques well known in the art. For example, samples can be assayed by Western blot analysis by adding 1×SDS loading buffer to the cells following treatment with the desired compound. After incubation at 95° C. for 10 min, samples are resolved onto polyacrylamide gel and transferred onto a PVDF membrane. Blots are probed with commercially available primary antibodies to one of the ovarian survival factor-associated molecules, such as MAGP2, to assess expression relative to a control sample not treated with the agents. Regardless of the assay technique, agents that cause at least a 2-fold decrease, such as at least a 3-fold decrease, at least a 4-fold decrease, or at least a 5-fold decrease in the activity, such as expression, of one or more the disclosed ovarian survival factor-associated molecules are selected for further evaluation.

Potential therapeutic agents identified with these or other approaches, including the specific assays and screening systems described herein, are used as lead compounds to identify other agents having even greater modulatory effects on the ovarian survival factor-associated molecules. For example, chemical analogs of identified chemical entities or siRNAs are tested for their activity in the assays described herein. For example, iSynthetic siRNA molecules are generated against selected target genes, such as any of the ovarian survival factor-associated upregulated genes identified in Tables 1 or 2. In an example, the siRNA molecules are obtained from commercial sources. Knockdown efficiency of the siRNA molecules is assessed as indicated in Example 1. In an example, a significant knockdown efficiency is approximately 20%. As provided in Example 1, the effects of target gene siRNA's on tumor growth and vascularization can be determined by evaluating the effect of siRNA treatment on cell migration, cell proliferation, cell adhesion and/or tube formation in desired cells, including ovarian tumor cell lines and HUVECs. In additional examples, cells are treated with two or more siRNAs (that target two or more genes). The IC₅₀ values are compared (between target gene siRNA individually and in combination) to determine whether the knockdown effect on tumor growth and vascularization is cumulative or additive.

Candidate agents also can be tested in additional cell lines and animal models of ovarian tumor or ovarian cancer to determine their therapeutic value. The agents also can be tested for safety in animals, and then used for clinical trials in animals or humans. In one example, genetically engineered mouse models of ovarian cancer are employed to determine therapeutic value of test agents. In a specific example, genetically engineered mouse models of epithelial ovarian cancer are utilized. For example, epithelial ovarian cancer can be induced in the mouse models by (a) inactivation of p53 and Rb, (b) induction of activated K-ras in the absence of Pten, or (c) induction of the transforming region of SV40 T antigen under transcriptional control of a portion of the murine Mullerian inhibiting substance type II receptor (MISIIR) gene promoter locally in the ovarian surface epithelial as previously described (Connolly et al., Cancer Res. 63:1389-97, 2003; Flesken-Nikitin et al., Cancer Res. 63: 3459-63, 2003; and Dinulescu et al., Nat. Med. 11:63-70, 2005). These are existing mouse models that are maintained independently in the laboratories of the investigators who generated them (Id.).

### Example 10

### Effectiveness of an Ovarian Tumor Treatment

This example describes methods that can be used to identify effective ovarian tumor treatments.

Based upon the teachings disclosed herein, the effectiveness of an ovarian tumor treatment can be evaluated by determining the effectiveness of an agent for the treatment of an ovarian tumor in a subject with the ovarian tumor. In an example, the method includes detecting expression of an ovarian survival factor-associated molecule in a sample from the subject following treatment with the agent. The expression of the ovarian survival factor-associated molecule following treatment is compared to a control. An alteration in the expression of the ovarian survival factor-associated molecule following treatment indicates that the agent is effective for the treatment of the ovarian cancer in the subject. For example, a decrease of at least 2-fold, at least 3-fold, or at least 5-fold of one or more of the disclosed ovarian survival factor-associated molecules, such as MAGP2, relative to control values (e.g., expression level in a subject without ovarian cancer or prior to receiving the treatment) indicates the treatment is an effective ovarian tumor treatment. In a specific example, the method includes detecting and comparing the protein expression levels of the ovarian survival factor-associated molecules by techniques described in detail above. In other examples, the method includes detecting and comparing the mRNA expression levels of the ovarian survival factor-associated molecules.

### Example 11

### Diagnosis and Prognosis of Ovarian Cancer

This example describes particular methods that can be used to diagnose or prognose an ovarian tumor in a subject, such as metastatic ovarian cancer in a human. However, one skilled in the art will appreciate that similar methods can be used. In some examples, such diagnosis is performed before treating the subject (for example as described in Example 8).

Biological samples are obtained from the subject. If blood or a fraction thereof (such as serum) is used 1-100 μl of blood is collected. Serum can either be used directly or fractionated using filter cut-offs to remove high molecular weight proteins. If desired, the serum can be frozen and thawed before use. If a tissue biopsy sample is used, 1-100 μg of tissue is obtained, for example using a fine needle aspirate RNA is isolated from the tissue using routine methods (for example using a commercial kit).

In one example, the diagnosis or prognosis of a metastatic ovarian tumor is determined by detecting pro-angiogenic ovarian survival factor-associated nucleic acid expression levels in a tumor sample obtained from a subject by microarray analysis or real-time quantitative PCR (as described in detail in Example 1). For example, the disclosed gene profile can be utilized. In other examples, the amount of such molecules is determined at the protein level by methods known to those of ordinary skill in the art, such as protein microarray, Western blot or immunoassay techniques. The relative amount of pro-angiogenic ovarian survival factor-associated molecules detected are compared to a reference value, such as a relative amount of such molecules present in a non-tumor sample from, wherein the presence of significantly more pro-angiogenic ovarian survival factor-associated molecules in the tumor sample as compared to the non-tumor sample (such as an increase of at least 2-fold, at least 3-fold, or at least 5-fold) indicates that the subject has a metastatic ovarian tumor, has an increased likelihood of an ovarian tumor metastasizing, has a poor prognosis, or combinations thereof. In some examples, relative amount of pro-angiogenic ovarian survival factor-associated proteins and pro-angiogenic ovarian survival factor-associated mRNA expression are determined in the same subject using the methods described above.

In other examples, ovarian survival factor-associated nucleic acid expression levels are determined in a tumor sample obtained from the subject by microarray analysis or real-time quantitative PCR to determine the prognosis. In an example, the disclosed gene profile is utilized. In other examples, the amount of such molecules is determined at the protein level by methods known to those of ordinary skill in the art, such as protein microarray, Western blot or immunoassay techniques. The relative amount of ovarian survival factor-associated molecules are compared to a reference value, such as a relative amount of such molecules present in a non-tumor sample from, wherein the presence of significantly more ovarian survival factor-associated molecules in the tumor sample as compared to the non-tumor sample (such as an increase of at least 2-fold, at least 3-fold, or at least 5-fold) indicates that the subject has a poor prognosis. A poor prognosis may include a decreased chance for survival (such a survival time of about one year or less), an increased likelihood of an ovarian tumor metastasizing, a decreased likelihood of responding to chemotherapy or combinations thereof. In some examples, relative amount of ovarian survival factor-associated proteins and ovarian survival factor-associated mRNA expression are determined in the same subject using the methods described above.

In additional examples, ovarian survival factor-associated protein levels are determined in a serum sample obtained from the subject. The serum sample described above is incubated with an antibody specific to one or more of the disclosed ovarian survival factor-associated molecules (e.g., a commercially available antibody to MAGP2) for a time sufficient for the antibody to bind to the ovarian survival factor-associated molecule (e.g., MAGP2) in the serum. The ovarian survival factor-associated molecule/antibody complexes are detected, for example using an ELISA. Alternatively, the serum sample is subjected to SDS-PAGE, and transferred to a membrane (such as nitrocellulose), which is probed with the desired antibody. The ovarian survival factor-associated molecule/antibody complexes can be detected with a secondary labeled antibody, or by observing the appropriated sized protein on the gel. The relative amount of ovarian survival factor-associated molecule/antibody complexes in the serum sample from the subject can be compared to a reference value, such as a relative amount of ovarian survival factor-associated molecule/antibody complexes present in a serum sample from a subject not having a tumor, wherein the presence of significantly more ovarian survival factor-associated molecule/antibody complexes in the test sample as compared to the reference sample (such as an increase of at least 2-fold, at least 3-fold, or at least 5-fold) indicates that the subject has an ovarian tumor, has a metastatic ovarian tumor, has a poor prognosis, or combinations thereof.

### Example 12

### Effect of MAGP2 siRNA on Ovarian Tumor Growth and Microvessel Densities in Vivo

This example illustrates the effect of MAGP2 siRNA on ovarian tumor growth and microvessel densities in vivo.

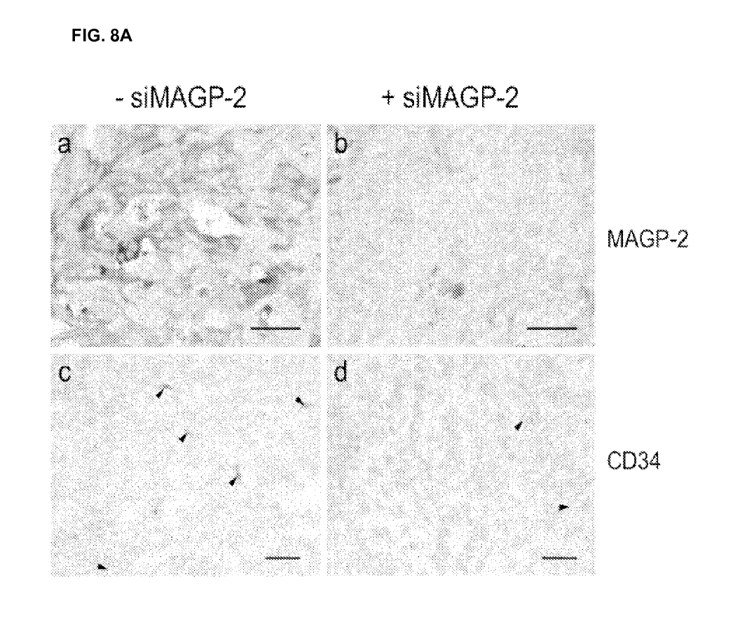

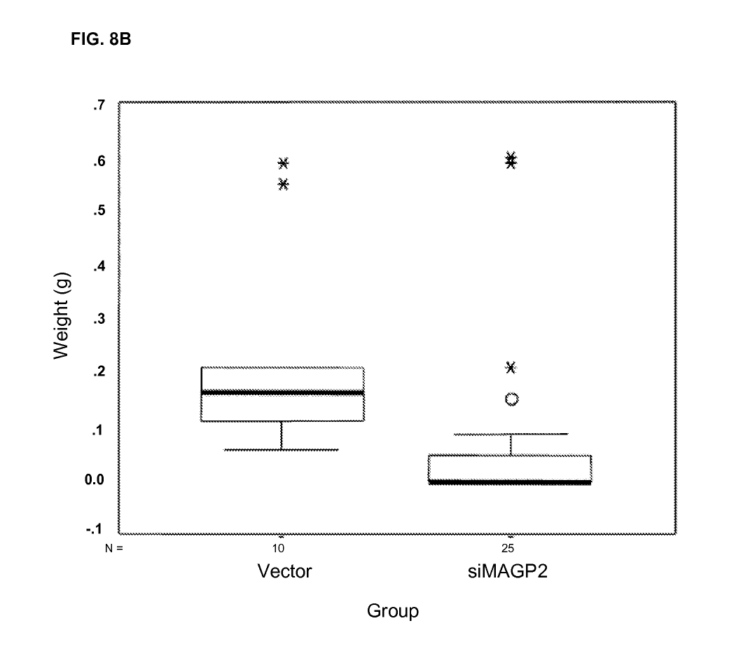

To investigate the effects of decreased MAGP2 expression on ovarian tumor growth and microvessel densities in vivo, ovarian cancer cells SKOV3 were first stably transfected with MAGP2 siRNA or the empty vector. The MAGP2 siRNA molecule had the following sequence: 5′-ACCGGTTAAACAATGCATTCAT-3′ (sense; SEQ ID NO: 1) and 5′-ATGAATGCATTGTTTAACCGGC-3′ (antisense; SEQ ID NO: 2). Five stable clones were selected for each group. A total of 5×10⁵ cells from each clone were injected subcutaneously into the posterior neck region of 5 nude mice (6 to 8 week-old female nude mice, Charles River, Mass.). After 4 weeks, the mice were sacrificed and the tumors developed from each mouse were removed and weighed. They samples were subsequently fixed in formalin and processed for histological evaluation. MAGP2 expression in tumor tissues was evaluated using a rabbit anti-human MAGP2 antibody and the microvessel density was determined by immunolocalization of CD34+ blood vessels using a goat anti-mouse polyclonal antibody (FIG. 8A). Significant difference in the weight of the tumors between the MAGP2 siRNA and the control group were found as determined by Mann-Witney U test (FIG. 8B). A p value <0.05 was considered as significant. This data indicates that MAGP2 plays a role in ovarian tumor vascularization and MAGP2 siRNA can significantly decrease the weight of an ovarian tumor in vivo.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims. We therefore claim as our invention all that comes within the scope and spirit of these claims.

1. A method of diagnosing or prognosing a subject with an ovarian tumor, comprising:
detecting expression of at least two ovarian survival factor-associated molecules listed in Table 1 or Table 2, each of which has a Cox hazard ratio of greater than 8, in a sample obtained from the subject with the ovarian tumor, thereby diagnosing or prognosing the subject.

2. The method of claim 1, further comprising comparing expression of the at least two ovarian survival factor-associated molecules in the sample obtained from the subject with the ovarian tumor to a control, wherein increased expression of the at least two ovarian survival factor-associated molecules relative to a control indicates that the subject has a decreased chance of survival.

3. The method of claim 1, wherein the at least two ovarian survival factor-associated molecules comprise microfibril-associated glycoprotein 2 (MAGP2), stanniocalcin 2 (STC2), chemokine (C-C motif) receptor-like 1 (CCRL1), klotho beta (KLB), protein tyrosine phosphatase receptor D (PTPRD) or matrix metallopeptidase 13 (MMP13).

4. The method of claim 1, wherein one of the at least two ovarian survival factor-associated molecules comprises MAGP2.

5. The method of claim 1, wherein the at least two ovarian survival factor-associated molecules comprise all of the ovarian survival factor-associated molecules listed in Table 2.

6. The method of claim 1, wherein the at least two ovarian survival factor-associated molecules comprise all of the ovarian survival factor-associated molecules listed in Table 1.

7.-11. (canceled)

12. The method of claim 1, wherein the at least two ovarian survival factor associated molecules listed in Table 1 or Table 2 has a Cox hazard ratio of greater than 10.

13. (canceled)

14. The method of claim 1, wherein a decreased chance of survival comprises a survival time of equal to or less than one year.

15. The method of claim 1, wherein no significant change in the expression of the at least two ovarian survival factor-associated molecules indicates an increase chance in survival.

16. (canceled)

17. The method of claim 1, wherein the method is used for diagnosing or prognosing a subject with an ovarian papillary serous cancer.

18. The method of claim 1, wherein the method used for prognosing a subjects response to chemotherapy, wherein an increase in the expression of the at least two ovarian survival factor-associated molecules indicates that the subject has a decreased chance of responding to a chemotherapeutic agent.

19. A method of treating an ovarian tumor in a subject, comprising:
administering to the subject a therapeutically effective amount of an agent that decreases biological activity of at least one ovarian survival factor-associated molecule listed in any of Tables 1 or 2 with a Cox hazard ratio of greater than 8, thereby increasing the subject's chance of survival.

20. The method of claim 19, wherein the agent reduces the biological activity of microfibril-associated glycoprotein 2 (MAGP2).

21. The method of claim 19, wherein the agent decreases expression of microfibril-associated glycoprotein 2 (MAGP2), stanniocalcin 2 (STC2), chemokine (C-C motif) receptor-like 1 (CCRL1), klotho beta (KLB), protein tyrosine phosphatase receptor D (PTPRD) or matrix metallopeptidase 13 (MMP13).

22. The method of claim 19, wherein the agent decreases expression of at least one ovarian survival factor-associated molecule with a Cox hazard ratio of greater than 10.

23.-25. (canceled)

26. The method of claim 19, wherein the agent reduces expression of the at least one ovarian survival factor-associated molecule listed in Table 1 or Table 2 relative to a control.

27. The method of claim 19, wherein the specific binding agent is a small inhibitory (si)RNA.

28. A method of identifying an agent for use in treating an ovarian tumor, comprising:
contacting an ovarian tumor epithelial cell with one or more test agents under conditions sufficient for the one or more test agents to decrease the activity of at least one ovarian survival factor-associated molecule listed in Table 1 or Table 2 with a Cox hazard ratio of greater than 8; and
detecting activity of the at least one ovarian survival factor-associated molecule in the presence of the one or more test agents; and
comparing activity of the at least one ovarian survival factor-associated molecule in the presence of the one or more test agents to a reference value to determine if there is expression of the at least one ovarian survival factor-associated molecule,
wherein decreased expression of the ovarian survival factor-associated molecule indicates that the one or more test agents is of use to treat the ovarian tumor.

29.-34. (canceled)

35. A method of determining the effectiveness of an agent for the treatment of an ovarian tumor in a subject with the ovarian tumor, comprising:
detecting expression of an ovarian survival factor-associated molecule in a sample from the subject following treatment with the agent; and
comparing expression of the ovarian survival factor-associated molecule following treatment to a reference value, wherein a decrease in the expression of the ovarian survival factor-associated molecule following treatment indicates that the agent is effective for the treatment of the ovarian cancer in the subject.

36. The method of claim 35, wherein the reference value represents an expression value of the ovarian survival factor-associated molecule in a sample from the subject prior to treatment with the agent.

37. The method of claim 35, wherein the ovarian survival factor-associated molecule comprises molecules listed in Table 1, Table 2, or FIG. 1A with a Cox hazard ratio of greater than 8.

38. A method of determining the metastatic potential of an ovarian tumor in a subject, comprising:
detecting expression of at least two ovarian survival factor-associated molecules listed in Table 1 or Table 2 with a Cox hazard ratio of greater than 8 in a sample obtained from a subject with an ovarian tumor, in which the at least two ovarian survival factor-associated molecules are involved in promoting angiogenesis; and
comparing expression of the at least two ovarian survival factor-associated molecules in the sample obtained from the subject with the ovarian tumor to a reference value, wherein an increase in the expression of the at least two ovarian survival factor-associated molecules involved in promoting angiogenesis indicates that the subject has an ovarian tumor with increased metastatic potential.

39. The method of claim 38, wherein one of the at least two ovarian survival factor-associated molecules comprise microfibril-associated glycoprotein 2 (MAGP2).

40. The method of claim 38, wherein an increase in the expression of the at least two ovarian survival factor-associated molecules comprising MAGP2 indicates that the subject has an ovarian tumor with increased metastatic potential and decreased responsiveness to a chemotherapeutic agent.
